(19) United States
(12) Patent Application Publication
(10) Pub. No.: US 2026/0137677 A1
(43) Pub. Date: May 21, 2026

(54) PROTACS USEFUL AGAINST THE MAIN PROTEASE SARS-COV-2

(71) Applicants: Tocris Cookson Limited, Bristol (GB); Helmholtz Zentrum für Infektionsforschung GmbH, Braunschweig (DE); Universität zu Lübeck, Lübeck (DE)

(72) Inventors: Hannah Maple, Bristol (GB); Graham Marsh, Bristol (GB); Alex Moloney, Bristol (GB); Burhan Karadogan, Bristol (GB); Mark Brönstrup, Braunschweig (DE); Thomas Siemon, Braunschweig (DE); Katharina Rox, Braunschweig (DE); Rolf Hilgenfeld, Lübeck (DE)

(21) Appl. No.: 19/118,988

(22) PCT Filed: Oct. 6, 2023
(86) PCT No.: PCT/GB2023/052603

§ 371 Date: Apr. 7, 2025

(30) Foreign Application Priority Data
Oct. 7, 2022 (GB) 2214796.1

(51) Int. Cl. A61K 31/496; A61K 31/4439; A61K 31/4545; A61K 31/4725; A61P 31/12; C07D 401/14; C07D 405/14; C07D 417/14

(52) U.S. Cl. CPC A61K 31/496; A61K 31/4439; A61K 31/4545; A61K 31/4725; A61P 31/12; C07D 401/14; C07D 405/14; C07D 417/14

(57) ABSTRACT

Compounds of formula (I) are disclosed: or a salt, solvate or tautomer thereof, wherein; M_{PL} comprises a SARS-CoV-₂ main protease ligand, X comprises a divalent exit vector; Xi comprises a divalent exit vector; a and b are independently selected from 1 or 2; L comprises a divalent linker, and U_{L} comprises an E3 ubiquitin ligase ligand. Also disclosed are pharmaceutical compositions comprising such compounds or combinations, and methods and reagents using the compounds. Compounds may have therapeutic uses and uses in research.

### FIELD OF THE INVENTION

The present disclosure relates to compounds, combinations comprising such compounds, pharmaceutical compositions comprising such compounds or combinations, and methods and reagents using the compounds.

### BACKGROUND TO THE INVENTION

The COVID-19 pandemic has had a significant impact on global health and the global economy. The coronavirus SARS-CoV-2 that caused the pandemic is therefore an important therapeutic target. Zhang et al. (Science; 2020; Vol 368, Issue 6489 pp. 409-412) determined the crystal structure of a key protein in the virus' life cycle: the main protease (Mpro; also called 3CLpro) which has an essential role in processing the polyproteins that are translated from the viral RNA. Inhibitors of the protein have been developed, with potent enzymatic inhibition and cellular antiviral activity (see, for example, Kitamura et al., Journal of Medicinal Chemistry 2022 65 (4), 2848-2865 and WO-A-2021/160220 disclosing substances having a broad-band action against 3C or 3C-like (3CL) proteases of RNA viruses).

Protein degradation is a useful therapeutic strategy. Targeted degradation of a protein can be achieved using chimeric molecules known as proteolysis-targeting chimeras (PROTACs) or heterobifunctional degrader molecules (“degraders”). These compounds consist of a target warhead (i.e. a ligand specific for the target of interest) linked to a ligand targeting an E3 ubiquitin ligase. The degrader interacts to induce the heterodimerization of the two bound proteins, thus resulting in the ubiquitination and subsequent proteasomal degradation of the target of interest.

This principle has been successfully applied to several targets, including kinases, and transcriptional enzymes. PROTACs have usually focused on cancer-related targets where degradation of the host proteins leads to more favorable phenotypic outcomes than inhibition of the target. U.S. Pat. No. 9,988,376 discloses compounds including PROTACs which are capable of inhibiting oestrogen receptor function including compounds which degrade the oestrogen receptor.

There have recently been attempts to direct degraders to viral proteins. CN114874204 discloses PROTAC molecule targeting SARS-CoV-2 3C protease and applications of the PROTAC molecule. Shaheer et al., Journal of Biomolecular Structure and Dynamics, 40 (21), 2022, pages 10905-10917, discloses computational approach to design protein degrader probes for the main protease of SARS-CoV-2. WO-A-2022/081827 discloses proteolysis targeting chimeras (PROTACs) that target the degradation of viral proteins including coronaviral papain-like protease (PLpro), and main protease (Mpro). WO-A-2021/231778 discloses compounds and methods useful for the modulation of coronavirus protease via ubiquitination and/or degradation.

de Wispelaere et al. (Nat. Comm un. 10, 3468; 2019) and WO-A-2020/069125 disclose PROTACs that induce proteasomal degradation of viral proteins using telaprevir, a reversible-covalent inhibitor that binds to the hepatitis C virus (HCV) protease active site which is linked to ligands that recruit the CRL4 CRBN ligase complex, yielding compounds that can both inhibit and induce the degradation of the HCV NS3/4A protease.

There is nevertheless a need for improved treatments and tools to investigate viruses, in particular coronaviruses.

The present invention seeks to address this need and to overcome problem(s) associated with the prior art.

### SUMMARY OF THE INVENTION

The present inventors have developed PROTACs useful against the main protease of SARS-CoV-2. The antiviral mechanism of action of the PROTACs may be driven by proteasomal degradation of the viral proteins.

The present invention accordingly provides in a first aspect a compound of formula (I):

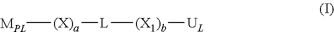

or a salt, solvate or tautomer thereof, wherein;
M_{PL} comprises a SARS-CoV-2 main protease ligand,
X comprises a divalent exit vector;
X1 comprises a divalent exit vector;
a and b are independently selected from 1 or 2;
L comprises a divalent linker, and
U_{L} comprises an E3 ubiquitin ligase ligand;
wherein M_{PL} is selected from:

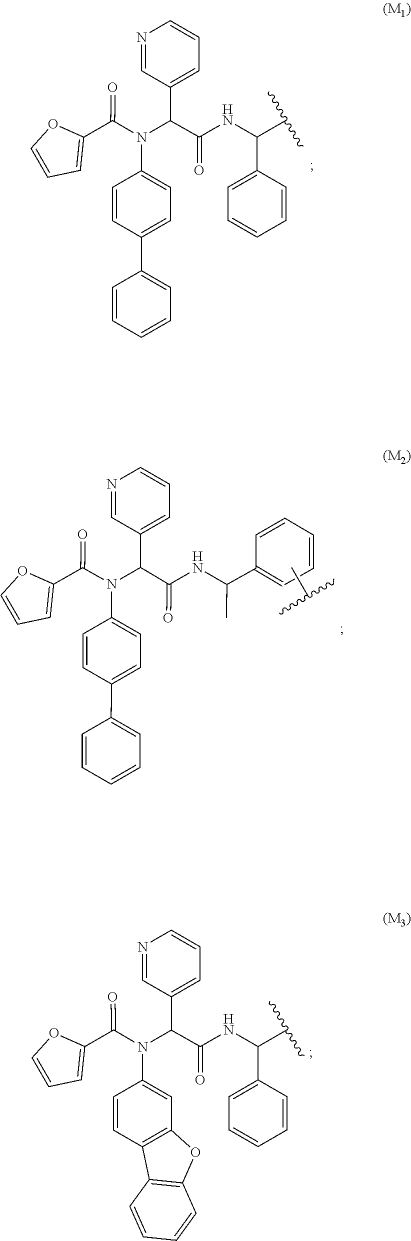

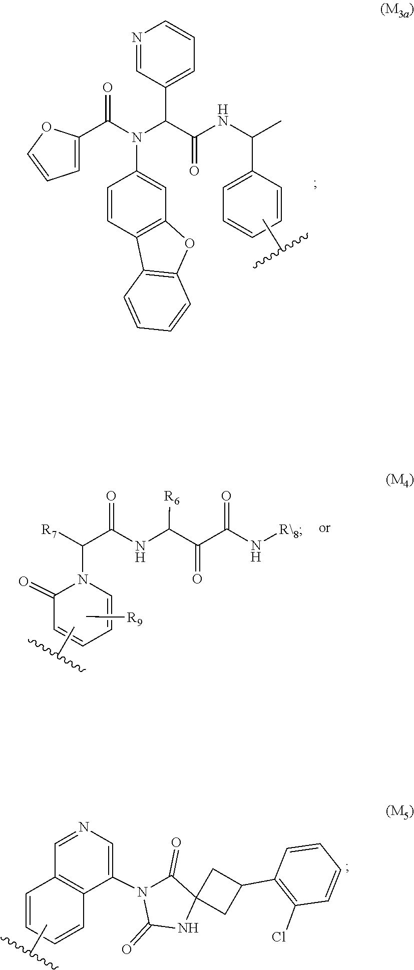

wherein
R₆ is (CH₂)_{c}— R₁₄,
R₇ is (CH₂)_{c}—R₁₅,
R₈ is (CH₂)_{c}—R₁₈,
R₉ is selected from H, C₁ to C₆ alkyl, halo, cyclopropyl, or phenyl,
R₁₄ is selected from a lactam that may be optionally substituted by 1 or 2 R₁₉ groups,
R₁₅ is selected from C₃ to C₆ cyclic alkyl or phenyl that may be optionally substituted by 1 or 2 R₁₉ groups,
R₁₈ is selected from H, C₁ to C₆ alkyl, halo, cyclopropyl that may be optionally substituted by a R₁₉ group, or phenyl that may be optionally substituted by 1, 2 or 3 Rig groups;
each c is independently 0, 1, or 2;
each R₁₉ is independently selected from C₁ to C₄ alkyl, or halo; and
the wavy line indicates the bond to the exit vector, X.

In some embodiments, one or more atoms in the compound of formula I may be isotopically substituted. For example, M1 may be:

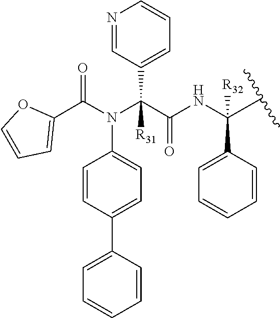

wherein R₃₁ and R₃₂ are independently selected from H or D.

R₁₅ may be a cyclic alkyl selected from cyclopropyl, cyclobutyl, cyclopentyl, or cyclohexyl that may be optionally substituted by 1 or 2 R₁₉ groups, or phenyl that may be optionally substituted by 1 or 2 R₁₉ groups. Suitably, R₁₅ may be cyclopropyl or cyclohexyl.

R₁₄ may be selected from a β (beta) lactam

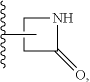

a γ (gamma) lactam

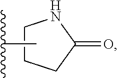

or a δ (delta) lactam

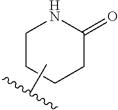

(each of which may be unsubstituted or optionally substituted by 1 or 2 R₁₉ groups).

Preferably, R₁₄ is a gamma lactam.

Preferably, c is 1.

M4 may be selected from:

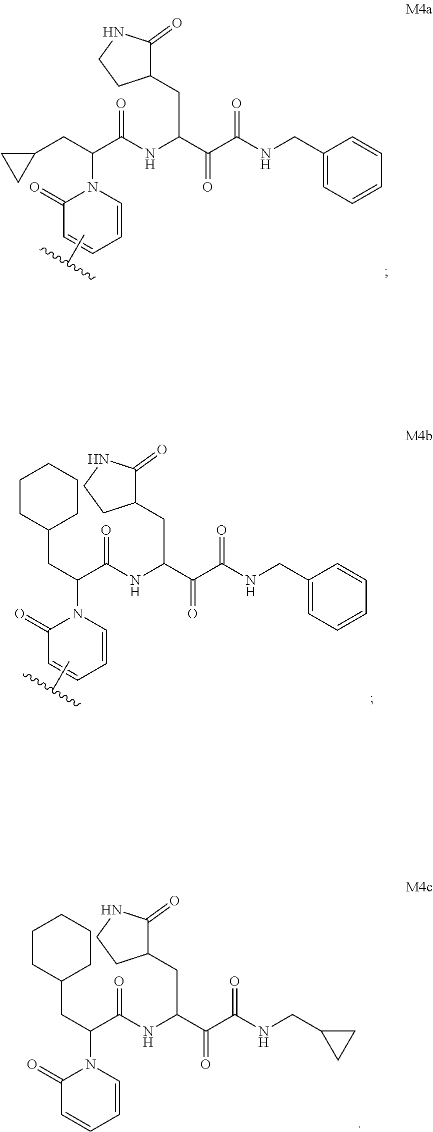

Advantageously, the mechanism of action of such PROTACs is event driven such that reduced binding affinity to a target will not necessarily lead to loss of target degradability providing the components are within functional proximity. PROTACs use a catalytic mechanism of action whereby one PROTAC can degrade multiple target proteins. Therefore PROTACs can be used at sub-stoichiometric concentrations relative to the target protein and the E3 ligase. This is greatly advantageous and means that PROTACs according to the invention may have improved function compared to inhibitors which typically rely on a 1:1 binding stoichiometry.

The locations of the exit vector on the M_{PL} structures are important in order to retain binding affinity for the main protease.

U_{L} comprises a ligand targeting a E3 ubiquitin ligase.

In some embodiments, a may be 1 and/or b may be 1.

If a and/or b is 2, each X (e.g. —X—X—) or X1 (e.g. —X1-X1-) may be selected independently.

U_{L} may comprise an E3 ubiquitin ligase targeting ligand selected from a ligand that targets cereblon (CRBN), Von Hippel-Lindau (VHL), inhibitor of apoptosis protein (IAP), mouse double minute 2 homolog (MDM2), or Kelch like ECH associated protein 1 (KEAP1).

Thus, U_{L} may comprise a ligand selected from a univalent substituent derived from VH101, VH032, VH298 or an immunomodulatory imide drug (IMiD), optionally thalidomide, lenalidomide, pomalidomide, avadomide; or 3-phenylpiperidine-2,6-dione.

U_{L} may be selected from a species of the following formulae:

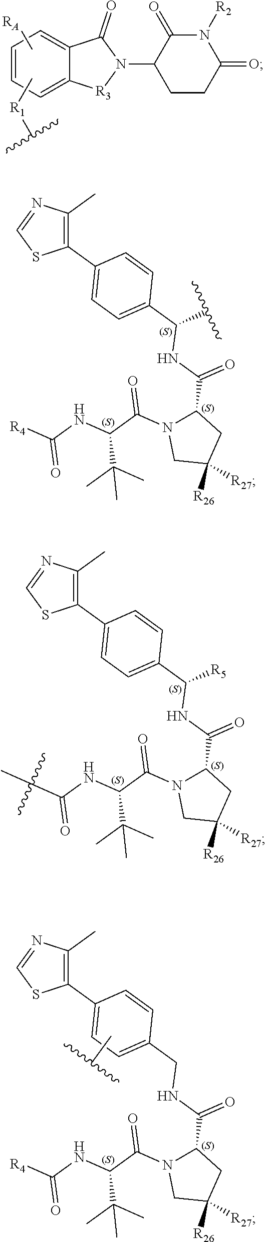

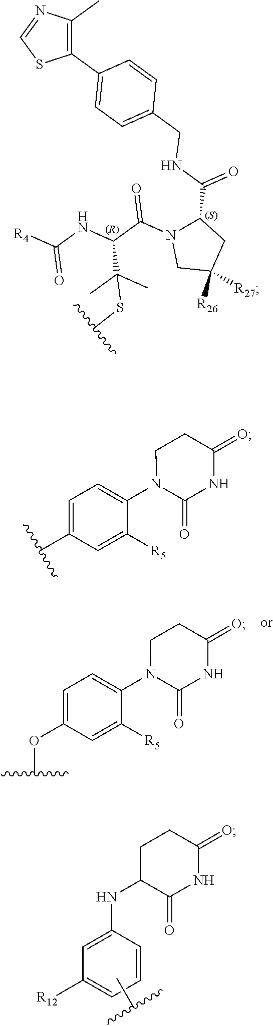

wherein R₁ is selected from —O—, —NH—, or —CH₂—, or is absent;
each R₂ or R₅ is independently selected from H or —CH₃;
each R₃ is independently selected from —CH₂— or —C(═O)—;
each R₄ is independently selected from —CH₃;

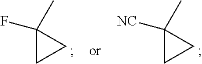

each R₂₆ and R₂₇ is selected from H, or OH; but R₂₆ and R₂₇ are not the same, R₁₂ is H or F,
each R_{A} is selected from H, C₁₋₆ alkyl, halo and (CH₂)ₜ—NR₂₀R₂₁,
t is selected from 0, 1, 2, or 3,
R₂₀ and R₂₁ are independently selected from H, and C₁₋₆ alkyl; and
the wavy line indicates the bond to X1.

Where the CRBN binder is N-methylated (i.e. on the glutarimide group), so that R₂ is CH₃, or R₂₆ is H and R₂₇ is OH, U_{L} may be a negative control.

Thus, in one aspect, U_{L} may be a group of formula:

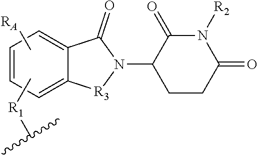

wherein R₂ is CH₃, and the other substituent are as indicated above.

Generally, however, R₂ may be H.

Generally, however, R₂₆ is OH and R₂₇ is H.

Suitably, U_{L} may be selected from a species of the following formulae:

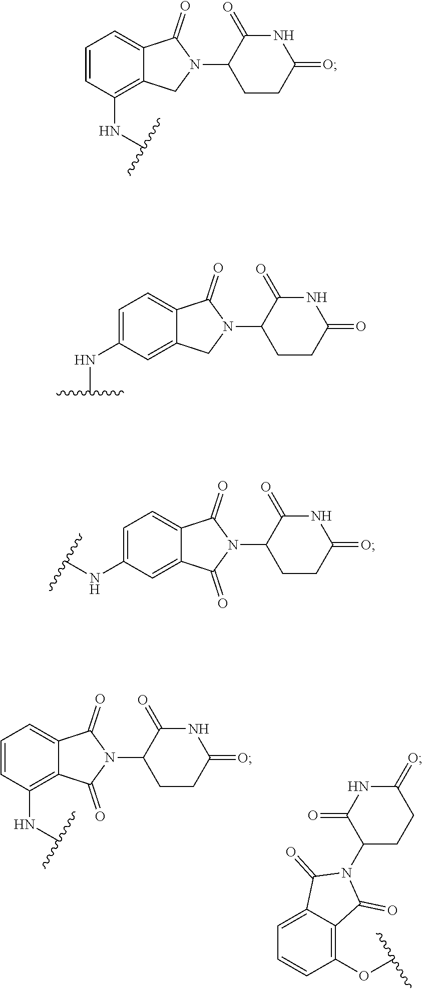

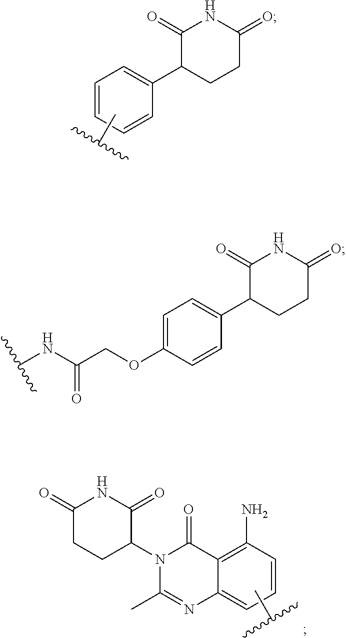

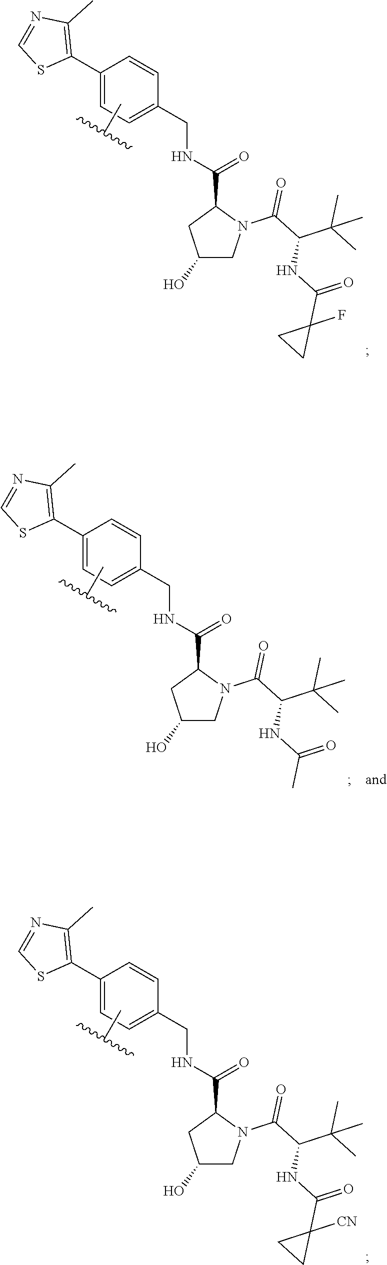

wherein R_{A} is selected from H, C₁₋₆ alkyl, halo and (CH₂)ₜ—NR₂₀R₂₁,
t is selected from 0, 1, 2, or 3,
R₂₀ and R₂₁ are independently selected from H, and C₁₋₆ alkyl; and
the wavy line indicates the bond to X1.

The exit vector X may each be such that each X is independently selected from a divalent substituent selected from a single covalent bond (e.g. is null or absent), —C(═O)—, —CH₂—, —O—, —S—, —C(═O)—NR₁₁—; —NR₁₁C(═O)—; —NR₁₁—; —C(═O)—O—; —O—CH₂—C(═O)—NR₁₁—; —NR₁₁—C(═O)—CH₂—O—; —C(═O)—CH₂—O—;

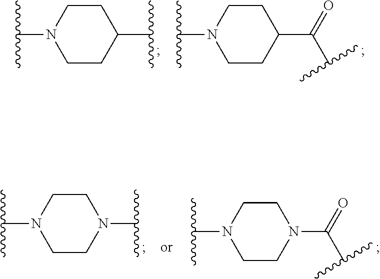

wherein each R₁₁ is independently selected from H, and C₁₋₆ alkyl, and
the bonds, or wavy lines in the formulae, indicate the bonds to M_{PL} and L or L and M_{PL}.

The exit vector may be bonded to M_{PL} and L in either orientation by either bond.

The linker may be flexible or rigid or partially rigid. Thus, L may be a divalent linking group comprising

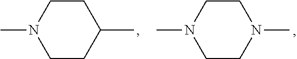

C₃₋₈ cycloalkyl, C₅₋₁₀ heteroarylene, phenylene, a C₁₋₁₂ alkylene chain which may contain one or more carbon-carbon double or triple bonds, a paraformaldehyde chain
—(CH₂CH₂)ₙ(CH₂)ₘ(OCH₂)ₛ(CH₂CH₂)ₚ(CH₂)_{q}—, a polyethylene glycol chain —(CH₂CH₂)ₙ(CH₂)ₘ(OCH₂CH₂)ᵥ(CH₂CH₂)ₚ(CH₂)_{q}—, which chains may be interrupted by one, two or three groups selected from —O—, —S—, —NH—, halo,

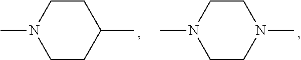

C₃₋₈ cycloalkyl, C₅₋₁₀ heteroarylene and/or phenylene; wherein n, m, p and q are independently 0, 1 or 2, s and v are independently 1 to 12, optionally 1 to 8, optionally 1 to 6.

The exit vector X1 may each be such that each X1 is independently selected from a single covalent bond (e.g. is null or absent), —C(═O)—, —CH₂—, —O—, —S—, —C(═O)—NR₁₁—; —NR₁₁C(═O)—; —NR₁₁—; —C(═O)—O—; —O—CH₂—C(═O)—NR₁₁—; —NR₁₁—C(═O)—CH₂—O—; —C(═O)—CH₂—O—;

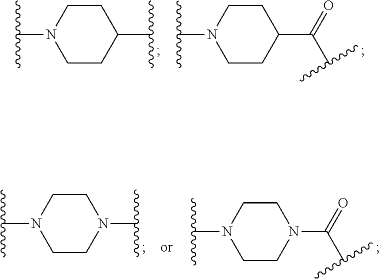

wherein each R₁₁ is independently selected from H, and C₁₋₆ alkyl, and the bonds or wavy lines in the formulae indicate the bonds to U_{L} and L or L and U_{L}. The exit vector may be bonded to U_{L} and L in either orientation by either bond.

Preferably, the compound may be selected from compounds of the formulae as set out in claim 10.

In a further aspect, there is provided a compound according to the first aspect, or a pharmaceutically acceptable salt thereof, for use as a medicament.

In a further aspect, there is provided a compound according to the first aspect, or a pharmaceutically acceptable salt thereof, for use as a medicament in the treatment of a viral disease.

In a further aspect, there is provided a compound according to the first aspect, or a pharmaceutically acceptable salt thereof, for use as a medicament in the treatment of a coronavirus disease.

In a further aspect, there is provided a compound according to the first aspect, or a pharmaceutically acceptable salt thereof, for use as a medicament in the treatment of SARS-CoV-2.

In a second aspect, there is provided a combination comprising a compound according to the first aspect, or a pharmaceutically acceptable salt thereof, and at least one other active agent.

In a third aspect, there is provided a pharmaceutical composition comprising a compound according to the first aspect, or a combination according to the second aspect, and a pharmaceutically acceptable excipient, carrier or diluent.

In a fourth aspect there is provided a method of treatment of a subject suffering from a viral disease, the method comprising administering to said subject a therapeutically effective amount of a compound according to the first aspect or a pharmaceutical composition according to the second aspect.

In a fifth aspect there is provided a method (preferably an in vitro method) comprising providing a composition comprising a compound according to the first aspect, and contacting the composition with a source of a viral protease, optionally corono-viral protease; optionally SAR-CoV-2 Mpro.

In a sixth aspect there is provided a reagent comprising a compound according to the first aspect and a solvent.

Further particular and preferred aspects are set out in the accompanying independent and dependent claims. Features of the dependent claims may be combined with features of the independent claims as appropriate, and in combinations other than those explicitly set out in the claims.

### Definitions

“Substituted”, when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

In this specification, unless the context otherwise suggests, divalent groups may be connected to other parts of the molecule by either bond, thus in either orientation by either bond.

“Optionally substituted” refers to a parent group which may be unsubstituted or which may be substituted with one or more substituents. Suitably, unless otherwise specified, when optional substituents are present, the optional substituted parent group comprises from one to three optional substituents. Where a group may be “optionally substituted with 1, 2 or 3 groups”, this means that the group may be substituted with 0, 1, 2 or 3 of the optional substituents. Suitably, the group is substituted with 1, 2 or 3 of the optional substituents. Where a group is “optionally substituted with one or two optional substituents”, this means that the group may be substituted with 0, 1 or 2 of the optional substituents. Suitably, the group may be optionally substituted with 0 or 1 optional substituents. In some aspects, suitably the group is not optionally substituted. In other aspects, suitably the group is substituted with 1 of the optional substituents.

Optional substituents may be selected from C₁₋₈ alkyl, C₂₋₇ alkenyl, C₂₋₇ alkynyl, C₁₋₁₂ alkoxy, C₅₋₂₀ aryl, C₃₋₁₀ cycloalkyl, C₃₋₁₀ cycloalkenyl, C₃₋₁₀ cycloalkynyl, C₃₋₂₀ heterocyclyl, C₃₋₂₀ heteroaryl, acetal, acyl, acylamido, acyloxy, amidino, amido, amino, aminocarbonyloxy, azido, carboxy, cyano, ether, formyl, guanidino, halo, hemiacetal, hemiketal, hydroxamic acid, hydroxyl, imidic acid, imino, ketal, nitro, nitroso, oxo, oxycarbonyl, oxycarboyloxy, sulfamino, sulfamyl, sulfate, sulfhydryl, sulfinamino, sulfinate, sulfino, sulfinyl, sulfinyloxy, sulfo, sulfonamido, sulfonamino, sulfonate, sulfonyl, sulfonyloxy, uredio groups. In some aspects, the optional substituents are 1, 2 or 3 optional substituents independently selected from OH, C₁₋₈ alkyl, OC₁₋₁₂ alkyl, and halogen. More suitably, the optional substituents are selected from OH, C₁₋₈ alkyl and OC₁₋₁₂ alkyl; more suitably, the optional substituents are selected from C₁₋₈ alkyl and OC₁₋₁₂ alkyl.

“Independently” or “Independently selected” is used in the context of statement that, for example, “each R₁₆, R₁₇, is independently H, C₁₋₈ alkyl, . . . ” and means that each instance of the functional group, e.g. R₁₆, is selected from the listed options independently of any other instance of R₁₆ or R₁₇ in the compound. Hence, for example, H may be selected for the first instance of R₁₆ in the compound; methyl may be selected for the next instance of R₁₆ in the compound; and ethyl may be selected for the first instance of R₁₇ in the compound.

“C₁₋₈ alkyl”: refers to straight chain and branched saturated hydrocarbon groups, generally having from 1 to 8 carbon atoms; suitably a C₁₋₇ alkyl; suitably a C₁₋₆ alkyl; suitably a C₁₋₅ alkyl; more suitably a C₁₄ alkyl; more suitably a C₁₋₃ alkyl. Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, n-octyl and the like.

“Alkylene” refers to a divalent radical derived from an alkane which may be a straight chain or branched, as exemplified by —CH₂CH₂CH₂CH₂—. The alkylene may have the number of carbons as discussed above for alkyl groups.

“C₆₋₂₆ aralkyl” refers to an arylalkyl group having 6 to 26 carbon atoms and comprising an alkyl group substituted with an aryl group. Suitably the alkyl group is a C₁₋₆ alkyl group and the aryl group is phenyl. Examples of C₆₋₂₆ aralkyl include benzyl and phenethyl. In some cases, the C₆₋₂₆ aralkyl group may be optionally substituted, and an example of an optionally substituted C₆₋₂₆ aralkyl group is 4-methoxylbenzyl.

“C₅₋₂₀ Aryl”: refers to fully unsaturated monocyclic, bicyclic and polycyclic aromatic hydrocarbons having at least one aromatic ring and having a specified number of carbon atoms that comprise their ring members (e.g., C₅₋₂₀ aryl refers to an aryl group having from 5 to 20 carbon atoms as ring members). The aryl group may be attached to a parent group or to a substrate at any ring atom and may include one or more non-hydrogen substituents unless such attachment or substitution would violate valence requirements. Suitably, a C₆₋₁₄ aryl is selected from a C₆₋₁₂ aryl, more suitably, a C₆₋₁₀ aryl. Examples of aryl groups include phenyl.

“Arylene” refers to a divalent radical derived from an aryl group, e.g. —C₆H₄— which is the arylene derived from phenyl.

“C₃₋₈ cycloalkyl” or “3- to 8-membered cycloalkyl” means a closed ring of carbon atoms having 3 to 8 carbon atoms, preferably 3 to 7 carbon atoms, more preferably 3 to 6 carbon atoms and encompasses, for example, cyclopropyl, cyclobutyl, cyclopentyl, cyclohexyl, cycloheptyl and cyclooctyl.

“C₃₋₈ cycloalkylene” or “3- to 8-membered cycloalkylene” refers to a divalent radical derived from a cycloalkyl group, e.g. —C₆H₁₀—.

“C₃₋₈ cycloalkenylene” refers to a divalent radical derived from a cycloalkenyl group, that is a carbocyclic group with one or more C═C, e.g. —C₆H₈—.

Halogen or halo: refers to a group selected from F, Cl, Br, and I. Suitably, the halogen or halo is F or Cl. In some aspects, suitably, the halogen is F. In other aspects, suitably the halogen is Cl.

“C₅₋₁₀ heteroaryl” or “5- to 10-membered heteroaryl”: refers to unsaturated monocyclic or bicyclic aromatic groups comprising from 5 to 10 ring atoms, whether carbon or heteroatoms, of which from 1 to 5 are ring heteroatoms. Suitably, any monocyclic heteroaryl ring has from 5 to 6 ring atoms and from 1 to 3 ring heteroatoms. Suitably each ring heteroatom is independently selected from nitrogen, oxygen, and sulfur. The bicyclic rings include fused ring systems and, in particular, include bicyclic groups in which a monocyclic heterocycle comprising 5 ring atoms is fused to a benzene ring.

The heteroaryl group may be attached to a parent group or to a substrate at any ring atom and may include one or more non-hydrogen substituents unless such attachment or substitution would violate valence requirements or result in a chemically unstable compound.

Examples of monocyclic heteroaryl groups include, but are not limited to, those derived from:
N₁: pyrrole, pyridine;
O₁: furan;
S₁: thiophene;
N₁O₁: oxazole, isoxazole, isoxazine;
N₂O₁: oxadiazole (e.g. 1-oxa-2,3-diazolyl, 1-oxa-2,4-diazolyl, 1-oxa-2,5-diazolyl, 1-oxa-3,4-diazolyl);
N₃O₁: oxatriazole;
N₁S₁: thiazole, isothiazole;
N₂: imidazole, pyrazole, pyridazine, pyrimidine, pyrazine;
N₃: triazole, triazine; and,
N₄: tetrazole.

Examples of heteroaryl which comprise fused rings, include, but are not limited to, those derived from:
O₁: benzofuran, isobenzofuran;
N₁: indole, isoindole, indolizine, isoindoline;
S₁: benzothiofuran;
N₁O₁: benzoxazole, benzisoxazole;
N₁S₁: benzothiazole;
N₂: benzimidazole, indazole;
O₂: benzodioxole;
N₂O₁: benzofurazan;
N₂S₁: benzothiadiazole;
N₃: benzotriazole; and
N₄: purine (e.g., adenine, guanine), pteridine;
“heteroarylene” refers to a divalent radical derived from a heteroaryl group (such as those described above) as exemplified by pyridinyl —[C₅H₃N]—. Heteroarylenes may be monocyclic, bicyclic, or tricyclic ring systems. Representative heteroarylenes, are not limited to, but may be selected from triazolylene, tetrazolylene, oxadiazolylene, pyridylene, furylene, benzofuranylene, thiophenylene, benzothiophenylene, quinolinylene, pyrrolylene, indolylene, oxazolylene, benzoxazolylene, imidazolylene, benzimidazolylene, thiazolylene, benzothiazolylene, isoxazolylene, pyrazolylene, isothiazolylene, pyridazinylene, pyrimidinylene, pyrazinylene, triazinylene, cinnolinylene, phthalazinylene, quinazolinylene, pyrimidylene, azepinylene, oxepinylene, and quinoxalinylene. Heteroarylenes are optionally substituted.

“C₆₋₁₆ heteroarylalkyl” refers to an alkyl group substituted with a heteroaryl group. Suitably the alkyl is a C₁₋₆ alkyl group and the heteroaryl group is C₅₋₁₀ heteroaryl as defined above. Examples of C₆₋₁₆ heteroarylalkyl groups include pyrrol-2-ylmethyl, pyrrol-3-ylmethyl, pyrrol-4-ylmethyl, pyrrol-3-ylethyl, pyrrol-4-ylethyl, imidazol-2-ylmethyl, imidazol-4-ylmethyl, imidazol-4-ylethyl, thiophen-3-ylmethyl, furan-3-ylmethyl, pyridin-2-ylmethyl, pyridin-2-ylethyl, thiazol-2-ylmethyl, thiazol-4-ylmethyl, thiazol-2-ylethyl, pyrimidin-2-ylpropyl, and the like.

“C₃₋₂₀ heterocyclyl”: refers to saturated or partially unsaturated monocyclic, bicyclic or polycyclic groups having ring atoms composed of 3 to 20 ring atoms, whether carbon atoms or heteroatoms, of which from 1 to 10 are ring heteroatoms. Suitably, each ring has from 3 to 8 ring atoms and from 1 to 4 ring heteroatoms (e.g., suitably C₃₋₅ heterocyclyl refers to a heterocyclyl group having 3 to 5 ring atoms and 1 to 4 heteroatoms as ring members). The ring heteroatoms are independently selected from nitrogen, oxygen, and sulphur.

As with bicyclic cycloalkyl groups, bicyclic heterocyclyl groups may include isolated rings, spiro rings, fused rings, and bridged rings. The heterocyclyl group may be attached to a parent group or to a substrate at any ring atom and may include one or more non-hydrogen substituents unless such attachment or substitution would violate valence requirements or result in a chemically unstable compound.

Examples of monocyclic heterocyclyl groups include, but are not limited to, those derived from:
N₁: aziridine, azetidine, pyrrolidine, pyrroline, 2H-pyrrole or 3H-pyrrole, piperidine, dihydropyridine, tetrahydropyridine, azepine;
O₁: oxirane, oxetane, tetrahydrofuran, dihydrofuran, tetrahydropyran, dihydropyran, pyran, oxepin;
S₁: thiirane, thietane, tetrahydrothiophene, tetrahydrothiopyran, thiepane;
O₂: dioxoiane, dioxane, and dioxepane;
O₃: trioxane;
N₂: imidazoiidine, pyrazolidine, imidazoline, pyrazoline, piperazine:
N₁O₁: tetrahydrooxazole, dihydrooxazole, tetrahydroisoxazole, dihydroisoxazole, morpholine, tetrahydrooxazine, dihydrooxazine, oxazine;
N₁S₁: thiazoline, thiazolidine, thiomorpholine;
N₂O₁: oxadiazine;
O₁S₁: oxathiole and oxathiane (thioxane); and
N₁O₁S₁: oxathiazine.

Examples of substituted monocyclic heterocyclyl groups include those derived from saccharides, in cyclic form, for example, furanoses, such as arabinofuranose, lyxofuranose, ribofuranose, and xylofuranse, and pyranoses, such as aliopyranose, altropyranose, glucopyranose, mannopyranose, gulopyranose, idopyranose, galactopyranose, and talopyranose.

### L

As discussed herein, L is a linker group. Suitably, L may comprise an alkylene chain, paraformaldehyde chain or polyethylene glycol chain is interrupted by one or more hetero-atoms (e.g., N, O and S) and/or one or more C₅₋₁₀ heteroarylene groups (e.g., pyrrolylene, pyrazolylene, pyrazolylene, 1,2,3-triazolylene, pyridinylene) and/or one or more phenylene groups. More suitably, the chains may be interrupted by from one to three hetero-atoms and/or from one to three C₅₋₁₀ heteroarylene groups and/or from one to three phenylene groups.

Suitably L is selected from an alkylene chain containing from 1 to 11 carbon atoms, from 1 to 10 carbon atoms, from 1 to 9 carbon atoms, from 1 to 8 carbon atoms, from 1 to 7 carbon atoms, from 1 to 6 carbon atoms, from 1 to 5 carbon atoms, from 1 to 4 carbon atoms, from 1 to 3 carbon atoms, which may contain one or more carbon-carbon double or triple bonds; a paraformaldehyde chain —(OCH₂)₁₋₁₂—, —(OCH₂)₁₋₁₁—, —(OCH₂)₁₋₁₀—, —(OCH₂)₁₋₉—, —(OCH₂)₁₋₈—, —(OCH₂)₁₋₇—, —(OCH₂)₁₋₆—, —(OCH₂)₁₋₅—, —(OCH₂)₁₋₄—, —(OCH₂)₁₋₃— a polyethylene glycol chain —(OCH₂CH₂)₁₋₅—, —(OCH₂CH₂)₁₋₄—, —(OCH₂CH₂)₁₋₃—; which chain may be interrupted by one or more hetero-atoms and/or C₅₋₉ heteroarylene groups and/or from one to three phenylene groups.

More suitably, L may be selected from an alkylene chain containing from 1 to 12 carbon atoms which may contain one or more carbon-carbon double or triple bonds.

More suitably, L may be selected from CH═CH, CH₂, CH₂CH₂, CH₂CH₂CH₂, CH₂CH₂CH₂CH₂ and CH₂CH₂CH₂CH₂CH₂.

L may be a moiety having 1-200 nonhydrogen atoms selected from C, N, O, S, or halogen, and optionally incorporates ether, oxo, carboxyl, carboxamide, carboxamidyl, urethanyl, branched, cyclic, unsaturated, amino acid, heterocyclyl, aryl or heteroaryl moieties. Linker L may be unbranched or branched, flexible or rigid, short or long and may incorporate any combination of moieties as deemed useful.

In some embodiments, at least a portion of the linker L may have a polyalkylene oxide polymeric region, which may enhance solubility of the compound of formula (I) or (II).

In some embodiments, the linker L may have a repeating unit of ethylene glycol, and may have a number of repeating ethylene glycol units of about 1 to about 6, or any number therebetween. In some embodiments, L may include about 3 to about 20, about 4 to about 15, about 5 to about 12 or about 6 to about 10 ethylene glycol units. In some embodiments, at least a portion of Linker L may include one or more amino acid moieties which may provide enhanced solubility for the compound of formula (I) or (II) Other polymeric types of moieties may be incorporated in the linker L, such as polyacids, polysaccharides, or polyamines. Other moieties such as substituted or unsubstituted cyclic, aromatic or heteroaromatic moieties may be used to enhance rigidity.

For example, the linker L can include ethylene glycol repeating units, and/or an amino acid sequence. In some embodiments, linker L₂ includes the formula:

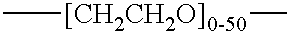

Any suitable number of ethylene glycol units can be used in the linker L of the present invention. For example, the linker L can include 1, 2, 3, 4, 5, 6 or more ethylene glycol units. In some embodiments, the linker L can include 5 ethylene glycol units. Several commercially available ethylene glycol groups (polyethylene glycol, PEG) are suitable in the linker L₂, such as H₂N-dPEG®₈-C(O)OH, having a discrete (“d”) polyethylene glycol having 8 ethylene glycol repeating units. Other discrete PEG units are commercially available and known to one of skill in the art, such as by Advanced ChemTech. In some embodiments, the linker L includes the formula:

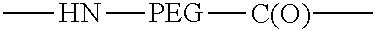

wherein PEG has 1-50 ethylene glycol units, usually 1 to 6 units.

The term “or pharmaceutically acceptable salts”, means that pharmaceutically acceptable salt, solvate, tautomeric, stereoisomeric forms of the shown structure are also included. “Mixtures thereof” means that mixture of these forms may be present, for example, the compounds of the invention may include both a tautomeric form and a pharmaceutically acceptable salt.

“Pharmaceutically acceptable” substances refers to those substances which are within the scope of sound medical judgment suitable for use in contact with the tissues of subjects without undue toxicity, irritation, allergic response, and the like, commensurate with a reasonable benefit-to-risk ratio, and effective for their intended use.

“Pharmaceutical composition” refers to the combination of one or more drug substances and one or more excipients.

As used herein, “solvate” refers to a complex of variable stoichiometry formed by a solute (e.g. formulas (1)-(1) (A), (B), (C), (D), or any other compound herein or a salt thereof) and a solvent. Pharmaceutically acceptable solvates may be formed for crystalline compounds wherein solvent molecules are incorporated into the crystalline lattice during crystallization. The incorporated solvent molecules can be water molecules or non-aqueous molecules, such as but not limited to, ethanol, isopropanol, dimethyl sulfoxide, acetic acid, ethanolamine, and ethyl acetate molecules.

The term “subject” as used herein refers to a human or non-human mammal. Examples of non-human mammals include livestock animals such as sheep, horses, cows, pigs, goats, rabbits and deer; and companion animals such as cats, dogs, rodents, and horses.

“Therapeutically effective amount” of a drug refers to the quantity of the drug or composition that is effective in treating a subject and thus producing the desired therapeutic, ameliorative, inhibitory, or preventative effect. The therapeutically effective amount may depend on the weight and age of the subject and the route of administration, among other things.

“Treating” refers to reversing, alleviating, inhibiting the progress of, or preventing a disorder, disease or condition to which such term applies, or to reversing, alleviating, inhibiting the progress of, or preventing one or more symptoms of such disorder, disease or condition.

“Treatment” refers to the act of “treating”, as defined immediately above.

As used herein the term “comprising” means “including at least in part of” and is meant to be inclusive or open ended. When interpreting each statement in this specification that includes the term “comprising”, features, elements and/or steps other than that or those prefaced by the term may also be present. Related terms such as “comprise” and “comprises” are to be interpreted in the same manner.

The term “consisting essentially of” limits the scope of a claim to the specified materials or steps “and those that do not materially affect the basic and novel characteristic(s)” of the claimed invention. When the phrase “consisting essentially of” appears in a clause of the body of a claim, rather than immediately following the preamble, it limits only the element set forth in that clause.

The term “consisting of” excludes any element, step, or ingredient not specified in the claim; “consisting of” defined as “closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole. It should be understood that while various embodiments in the specification are presented using “comprising” language, under various circumstances, a related embodiment is also described using “consisting essentially of” or “consisting of” language.

### Administration & Dose

Compounds of formula I may be administered alone or in combination with one or another or with one or more pharmacologically active compounds which are different from the compounds of formula I.

Compounds of the invention may suitably be combined with various components to produce compositions of the invention. Suitably the compositions are combined with a pharmaceutically acceptable carrier or diluent to produce a pharmaceutical composition (which may be for human or animal use). Suitable carriers and diluents include isotonic saline solutions, for example phosphate-buffered saline. Useful pharmaceutical compositions and methods for their preparation may be found in standard pharmaceutical texts. See, for example, Handbook for Pharmaceutical Additives, 3rd Edition (eds. M. Ash and I. Ash), 2007 (Synapse Information Resources, Inc., Endicott, New York, USA) and Remington: The Science and Practice of Pharmacy, 21st Edition (ed. D. B. Troy) 2006 (Lippincott, Williams and Wilkins, Philadelphia, USA) which are incorporated herein by reference.

The compounds of the invention may be administered by any suitable route. Suitably the compounds of the invention will normally be administered orally or by any parenteral route, in the form of pharmaceutical preparations comprising the active ingredient, optionally in the form of a non-toxic organic, or inorganic, acid, or base, addition salt, in a pharmaceutically acceptable dosage form.

The compounds of the invention, their pharmaceutically acceptable salts, and pharmaceutically acceptable solvates of either entity can be administered alone but will generally be administered in admixture with a suitable pharmaceutical excipient diluent or carrier selected with regard to the intended route of administration and standard pharmaceutical practice.

For example, the compounds of the invention or salts or solvates thereof can be administered orally, buccally or sublingually in the form of tablets, capsules (including soft gel capsules), ovules, elixirs, solutions or suspensions, which may contain flavouring or colouring agents, for immediate-, delayed-, modified-, sustained-, controlled-release or pulsatile delivery applications. The compounds of the invention may also be administered via fast dispersing or fast dissolving dosages forms.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and tale may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol.

The compounds of the invention can also be administered parenterally, for example, intravenously, intra-arterially, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Suitably formulation of the invention is optimised for the route of administration e.g. oral, intravenously, etc.

Administration may be in one dose, continuously or intermittently (e.g. in divided doses at appropriate intervals) during the course of treatment. Methods of determining the most effective means and dosage are well known to a skilled person and will vary with the formulation used for therapy, the purpose of the therapy, the target cell(s) being treated, and the subject being treated. Single or multiple administrations can be carried out with the dose level and the dose regimen being selected by the treating physician, veterinarian, or clinician.

Depending upon the disorder and patient to be treated, as well as the route of administration, the compositions may be administered at varying doses. For example, a typical dosage for an adult human may be 100 ng to 25 mg (suitably about 1 μg to about 10 mg) per kg body weight of the subject per day.

Suitably guidance may be taken from studies in test animals when estimating an initial dose for human subjects. For example when a particular dose is identified for mice, suitably an initial test dose for humans may be approx. 0.5× to 2× the mg/Kg value given to mice.

### Other Forms

Unless otherwise specified, included in the above are the well-known ionic, salt, solvate, and protected forms of these substituents. For example, a reference to carboxylic acid (—RCOOH) also includes the anionic (carboxylate) form (—RCOO—), a salt or solvate thereof, as well as conventional protected forms. Similarly, a reference to an amino group includes the protonated form (—RN+HR₁R₂), a salt or solvate of the amino group, for example, a hydrochloride salt, as well as conventional protected forms of an amino group. Similarly, a reference to a hydroxyl group also includes the anionic form (—O—), a salt or solvate thereof, as well as conventional protected forms.

### Isomers, Salts and Solvates

Certain compounds may exist in one or more particular geometric, optical, enantiomeric, diasteriomeric, epimeric, atropic, mesomeric stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; alpha- and beta-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH.

A reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not apply to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference in this specification to a particular compound includes all such isomeric forms, including (wholly or partially) racemic and other mixtures thereof.

Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

In some embodiments, the compound of formula (I) and salts and solvates thereof, comprises pharmaceutically acceptable salts of the compounds of formula (I).

Compounds of formula (I), which include compounds specifically named above, may form pharmaceutically acceptable complexes, salts, solvates and hydrates. These salts include nontoxic acid addition salts (including di-acids) and base salts.

If the compound is cationic, or has a functional group which may be cationic (e.g. —NH₂ may be —NH₃⁺), then an acid addition salt may be formed with a suitable anion.

Examples of suitable inorganic anions include, but are not limited to, those derived from the following inorganic acids hydrochloric acid, nitric acid, nitrous acid, phosphoric acid, sulfuric acid, sulphurous acid, hydrobromic acid, hydroiodic acid, hydrofluoric acid, phosphoric acid and phosphorous acids. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfonate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

For example, if the compound is anionic, or has a functional group which may be anionic (e.g. —RCOOH may be —RCOO—), then a base salt may be formed with a suitable cation. Examples of suitable inorganic cations include, but are not limited to, metal cations, such as an alkali or alkaline earth metal cation, ammonium and substituted ammonium cations, as well as amines. Examples of suitable metal cations include sodium (Na⁺) potassium (K⁺), magnesium (Mg²⁺), calcium (Ca²⁺), zinc (Zn²⁺), and aluminum (Al₃⁺). Examples of suitable organic cations include, but are not limited to, ammonium ion (i.e. NH₄⁺) and substituted ammonium ions (e.g. NH₃R₊, NH₂R₂₊, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺. Examples of suitable amines include arginine, N,N′-dibenzylethylene-diamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2011) Pharmaceutically acceptable salts may be prepared using various methods. For example, one may react a compound of formula 1 with an appropriate acid or base to give the desired salt. One may also react a precursor of the compound of Formula I with an acid or base to remove an acid- or base-labile protecting group or to open a lactone or lactam group of the precursor. Additionally, one may convert a salt of the compound of Formula 1 to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

It may be convenient or desirable to prepare, purify, and/or handle a corresponding solvate of the active compound. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, acetone-d6, DMSO-d6).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

### BRIEF DESCRIPTION OF THE DRAWINGS

Embodiments of the present invention will now be described further, with reference to the accompanying drawings, in which:

FIG. 1 shows in (a) the structure of the PROTAC according to the invention TS449F1; (b) the parent inhibitor compound 23R; graphs of results of inhibitory potency studies using a binary IC50 in vitro inhibitory assay against SARS-CoV-2 Mpro for the (c) PROTAC and (d) parent compound.

FIG. 2 shows in (a) the structure of the PROTAC according to the invention TS365F1; (b) the structure of the PROTAC according to the invention TS355F1; graphs of results of inhibitory potency studies using a binary IC50 in vitro inhibitory assay against SARS-CoV-2 Mpro for (c) 365 and (d) 355.

FIG. 3 shows in (a) the structure of the PROTAC according to the invention BT153; (b) the parent inhibitor compound 13bK; graphs of results of inhibitory potency studies using a binary IC50 in vitro inhibitory assay against SARS-CoV-2 Mpro for the (c) PROTAC 153 and (d) parent compound.

FIG. 4 shows in vitro biology of PROTACs. (A). BT153 shows an EC50 of 3.2 μM in an antiviral assay with VeroE6 infected with SARS-CoV-2 (virus strain SARS-CoV2/ZG/297-20, University Hospital for Infectious Diseases, Zagreb, Croatia, see: https://doi.org/10.1101/2020.07.09.195040) at an MOI of 0.05. (B). A plaque reduction assay performed in Vero E6 cells with BT153 demonstrates efficient reduction of viable virus.

FIG. 5 shows in vitro biology of PROTACs. (A) Chemical structures of selected C10778-series PROTACs, C10778N, C10778P, C10778R. (B) Antiviral assays with Vero E6 cells infected with SARS-CoV-2 at an MOI of 0.05, following dosing with the compounds shown in (A). (C) A plaque reduction assay performed in Vero E6 cells demonstrating that C10778N efficiently reduces viable virus.

### DESCRIPTION OF THE PREFERRED EMBODIMENTS

An advantage of PROTACs according to the invention, is that they may not bind within the active site of a protein where loss of efficacy mutations can occur. Typical antiviral small molecules inhibitors employ an occupancy driven mechanism of action where the binding affinity plays a large part in the efficacy of the compound. For example, in small molecule inhibitors against HIV protease, it has been shown that the accumulation of several mutations results in drug resistance to clinically approved compounds. PROTACs operate via an event driven process whereby sub-stoichiometric amounts of the degrader can be used because the process of degradation is catalytic and PROTACs are recycled. Further, mutations that reduce the affinity of the PROTAC for the target may still lead to degradation of the target providing the compound still induces proximity between the E3 ligase and the target protein. An additional benefit of antiviral PROTACs is that the portion of the compound binding to the target protein does not need to be within the active site which is often the site where resistance mutations are present. An antiviral PROTAC could bind to a location on the target that is distal to the active site and still lead to effective degradation.

### Synthetic Strategies

The compounds of Formula (I) may be prepared using the techniques described below. Some of the schemes and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in a number of treatises, including Richard Larock, Comprehensive Organic Transformations, A Guide to Functional Group Preparations, 2nd Ed (2010), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a diacid, etc.). In addition, in some instances, reaction intermediates may be used in subsequent steps without isolation or purification (i.e., in situ).

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry, 4th Edition, (2006) and P. Kocienski, Protective Groups, 3rd Edition (2005).

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78° C. to 0° C.). Any reference in the disclosure to a stoichiometric range, a temperature range, a pH range, etc., whether or not expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1,-dioxide); and phosphorus-containing solvents (e.g., HMPA, hexamethylphosphoramide).

### EXAMPLES

The isonitrile orthoester building block was synthesized adapting a procedure from the literature [J. Org. Chem. 2009, 74, 884-887].

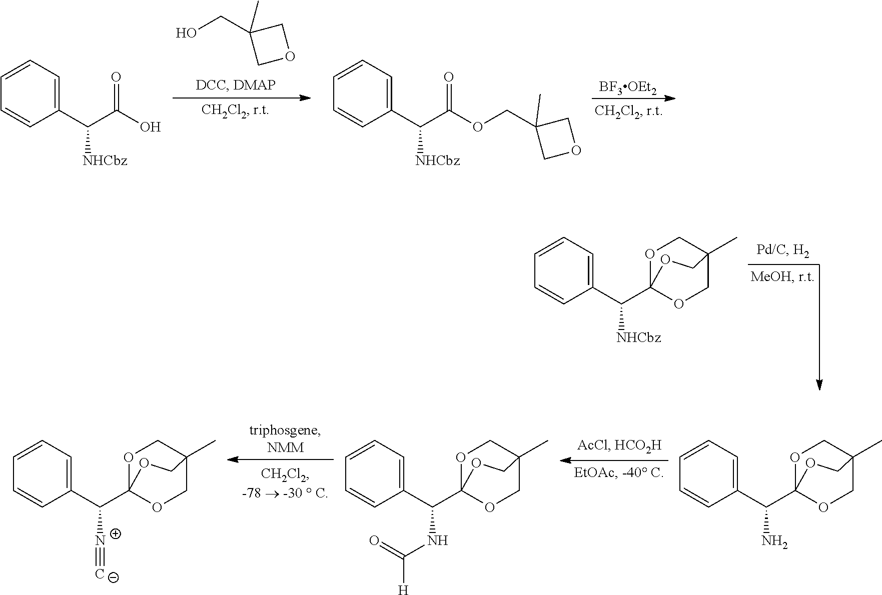

Step 1: To a solution of 3-Methyl-3-oxetanemethanol (4.30 g, 42.1 mmol, 1.2 eq.), DCC (7.30 g, 35.4 mmol, 1.01 eq.) and DMAP (42.8 mg, 0.35 mmol, 0.01 eq.) in CH₂Cl₂ (200 mL) was added Cbz-phenyl glycine (10.0 g, 35.0 mmol, 1.0 eq.) in portions at 0° C. The reaction mixture was stirred at room temperature for 1 h, before the precipitate was filtered off. The filtrate was washed with H₂O, 1 M HCl and brine. The organic phase was dried over Na₂SO₄ and concentrated in vacuo. The residue was dissolved in EtOAc and filtered through a short plug of silica to afford the oxetane (12.9 g, 99%) as a white solid. m/z (ESI): 370.2 [M+H⁺]⁺

Step 2: To a solution of the oxetane (12.9 g, 34.9 mmol, 1.0 eq.) in CH₂Cl₂ (100 mL) was added BF₃·OEt₂ (0.44 mL, 3.49 mmol, 0.1 eq.) and the reaction mixture was stirred at room temperature until complete conversion was achieved. Et₃N (0.73 mL, 5.23 mmol, 0.15 eq.) was added and the reaction mixture was concentrated in vacuo. The residue was dissolved in EtOAc (250 mL) and washed with sat. K₂CO₃ and brine. The organic phase was dried over Na₂SO₄ and concentrated in vacuo. The crude product was purified by recrystallization from EtOAc/hexane to afford the orthoester (8.61 g, 67%) as a white crystalline solid. ¹H NMR (500 MHz, CDCl₃) δ 7.42-7.27 (m, 10H), 5.68 (d, J=8.9 Hz, 1H), 5.10 (d, J=12.1 Hz, 1H), 5.05 (d, J=12.2 Hz, 1H), 4.92 (d, J=8.9 Hz, 1H), 3.89 (s, 6H), 0.79 (s, 3H). m/z (ESI): 370.2 [M+H⁺]⁺

Step 3: To a solution of the orthoester (8.61 g, 23.3 mmol, 1.0 eq.) in MeOH (200 mL) was added Pd/C (10% Pd, 2.48 g, 2.33 mmol, 0.1 eq.). The atmosphere was exchange by three times evacuation and purging with H₂. The reaction was stirred at room temperature overnight under an H₂ atmosphere. After complete conversion, the reaction mixture was filtered through Celite and washed thoroughly with MeOH. The filtrate was concentrated in vacuo to afford the amine (5.32 g, 97%) as a white solid.

Step 4: To a solution of acetyl chloride (1.86 mL, 26.0 mmol, 1.15 eq.) and formic acid (1.02 mL, 27.1 mmol, 1.2 eq.) in dry EtOAc (50 mL) was added Et₃N (7.80 mL, 56-5 mmol, 2.5 eq.) dropwise keeping the internal temperature between −60 to −78° C. A white precipitate is forming. The temperature was raised to −30° C. and a solution of the starting material (5.32 g, 22.6 mmol, 1.0 eq.) in dry EtOAc (20 mL) was added dropwise. After 10 min, TLC analysis indicated complete conversion of the starting material. The white precipitate was filtered off and washed thoroughly with EtOAc. The filtrate was concentrated in vacuo and dissolved in CH₂Cl₂. The organic phase was washed with sat. K₂CO₃ (+a little amount of 1 M KOH), dried over Na₂SO₄ and concentrated in vacuo to afford the formamide (5.67 g, 95%) as a white solid. ¹H NMR (500 MHz, CDCl₃) δ 8.21 (dd, J=1.7, 0.9 Hz, 1H), 7.43-7.24 (m, 5H), 6.45 (d, J=9.0 Hz, 1H), 5.24 (d, J=8.8 Hz, 1H), 3.90 (s, 6H), 0.80 (s, 3H). m/z (ESI): 264.2 [M+H⁺]⁺

Step 5: To a solution of the formamide (5.67 g, 21.5 mmol, 1.0 eq.) and N-methylmorpholine (4.80 mL, 43.1 mmol, 2.0 eq.) in CH₂Cl₂ (50 mL) was added triphosgene (2.23 g, 7.54 mmol, 0.35 eq.) in portions at −78° C. The reaction mixture was allowed to warm to −30° C. and stirred at this temperature until complete conversion of the starting material was observed by TLC analysis. The reaction was quenched by the addition of sat NH₄Cl (50 mL) and extracted with CH₂Cl₂ (3×50 mL). The combined organic phases were dried over Na₂SO₄ and concentrated in vacuo. Purification by flash column chromatography (silica gel, 50% EtOAc/cyclohexane) afforded the isonitrile (3.17 g, 62%) as a white solid. ¹H NMR (500 MHz, CDCl₃) δ 7.39-7.35 (m, 2H), 7.32-7.26 (m, 3H), 4.71 (s, 1H), 3.85 (s, 6H), 0.73 (s, 3H).

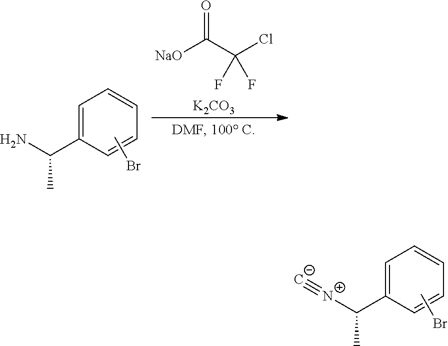

A solution of bromo phenyl glycine (1.0 eq.), sodium chlorodifluoroacetate (2.0 eq.) and K₂CO₃ (2.0 eq.) in DMF (0.1-0.2 M) is stirred at 100° C. overnight under an inert atmosphere. The reaction is quenched with H₂O at room temperature and extracted with CH₂Cl₂ (3×). The combined organic phases are dried over Na₂SO₄ and concentrated in vacuo. Purification by flash column chromatography (silica gel) affords the desired isonitrile.

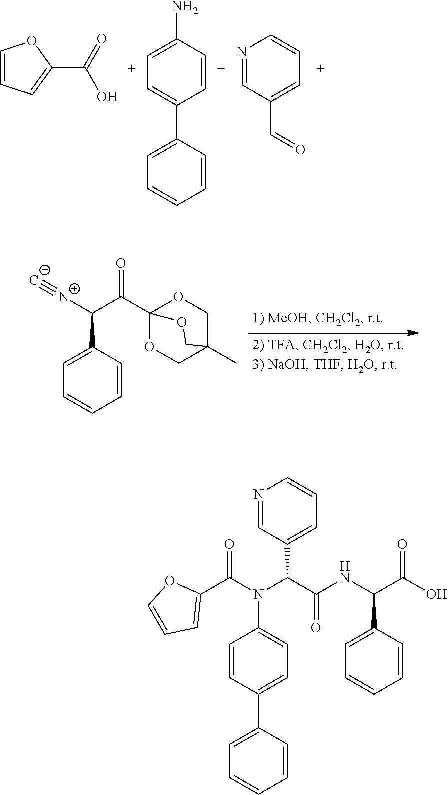

Step 1: Biphenylamine (830 mg, 4.90 mmol, 1.0 eq.) and 3-pyridinecarboxaldehyde (0.46 mL, 4.90 mmol, 1.0 eq.) were stirred in MeOH (50 mL) for 30 min upon which a precipitate had formed. 2-Furoic acid (550 mg, 4.90 mmol, 1.0 eq.) and a solution of the isonitrile (1.20 g, 4.90 mmol, 1.0 eq.) in CH₂Cl₂ (5 mL) were added and the reaction mixture was stirred overnight at room temperature. The reaction mixture was concentrated in vacuo. Purification by flash column chromatography (silica gel, 10-20% acetone/CH₂Cl₂) afforded the intermediate (1.57 g, 52%) as a slightly yellow oil. m/z (ESI): 616.4 [M+H⁺]⁺

Step 2: To a solution of the intermediate (1.57 g, 2.56 mmol, 1.0 eq.) in CH₂Cl₂ (25 mL) and H₂O (0.5 mL) was added TFA (1.0 mL) and the reaction mixture was stirred at room temperature for 1 h. The reaction was concentrated in vacuo and the residue was dissolved in THF (15 mL). An aqueous NaOH solution (1 M, 12.8 mL, 5.0 eq.) was added and the reaction was stirred at room temperature for 1 h. The mixture was acidified with 1 M HCl and extracted with EtOAc (3×25 mL). The combined organic phases were washed with brine, dried over Na₂SO₄ and concentrated in vacuo. The crude product was purified by RP-HPLC (35-65% MeCN/H₂O+0.1% formic acid) to afford the desired carboxylic acid diastereomer (RR, 380 mg, 28%) as well as the undesired diastereomer (SR, 535 mg, 39%) as white solids.

RR-Diastereomer: ¹H NMR (500 MHz, DMSO-d₆) δ 12.84 (s, 1H), 9.06 (d, J=6.5 Hz, 1H), 8.43 (d, J=2.3 Hz, 1H), 8.34 (dd, J=4.8, 1.7 Hz, 1H), 7.69 (d, J=1.6 Hz, 1H), 7.65-7.62 (m, 2H), 7.60-7.50 (m, 3H), 7.46-7.33 (m, 10H), 7.18 (dd, J=7.9, 4.8 Hz, 1H), 6.50 (s, 1H), 6.33 (dd, J=3.6, 1.7 Hz, 1H), 5.50 (d, J=3.5 Hz, 1H), 5.38 (d, J=6.4 Hz, 1H). m/z (ESI): 532.4 [M+H⁺]⁺

SR-Diastereomer: ¹H NMR (500 MHz, DMSO) δ 13.07 (s, 1H), 9.14 (d, J=7.4 Hz, 1H), 8.29 (dd, J=4.8, 1.6 Hz, 1H), 8.27 (d, J=2.2 Hz, 1H), 7.71 (d, J=1.7 Hz, 1H), 7.64-7.59 (m, 2H), 7.58-7.49 (m, 2H), 7.42 (dd, J=8.4, 7.0 Hz, 2H), 7.37-7.32 (m, 2H), 7.30-7.19 (m, 7H), 7.07 (dd, J=7.9, 4.8 Hz, 1H), 6.54 (s, 1H), 6.35 (dd, J=3.6, 1.7 Hz, 1H), 5.53 (d, J=3.5 Hz, 1H), 5.46 (d, J=7.4 Hz, 1H). m/z (ESI): 532.4 [M+H⁺]⁺

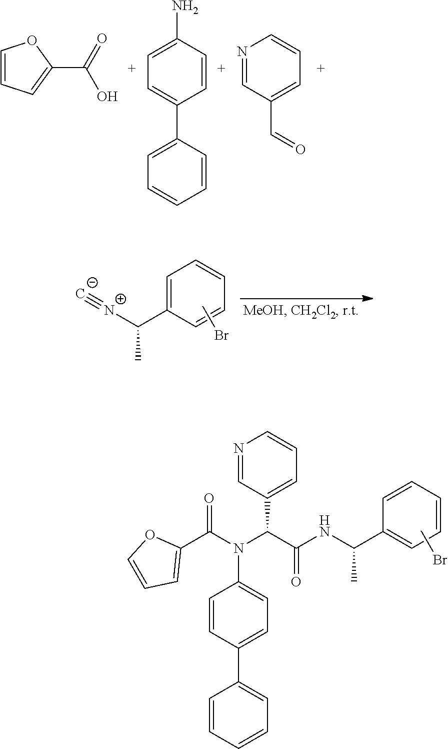

Biphenylamine (1.0 eq.) and 3-pyridinecarboxaldehyde (1.0 eq.) are stirred in MeOH for 30 min upon which a precipitate forms. 2-Furoic acid (1.0 eq.) and a solution of bromo (1-isocyanoethyl)benzene (1.0 eq.) in CH₂Cl₂ are added and the reaction mixture is stirred overnight at room temperature. The reaction mixture is concentrated in vacuo. Purification by flash column chromatography (silica gel) affords the product.

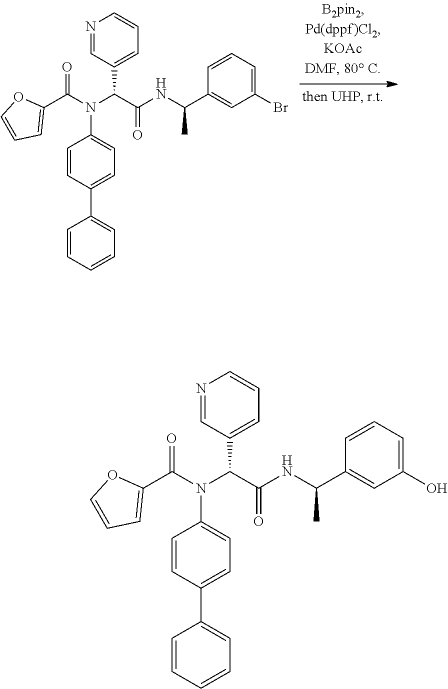 [SMILES: C[C@@H](NC(=O)[C@@H](c1cccnc1)N(C(=O)c1ccco1)c1ccc(-c2ccccc2)cc1)c1cccc(Br)c1 ; C[C@@H](NC(=O)[C@@H](c1cccnc1)N(C(=O)c1ccco1)c1ccc(-c2ccccc2)cc1)c1cccc(O)c1]

To a solution of the bromide (300 mg, 0.52 mmol, 1.0 eq.), bis(pinacolato)diboron (328 mg, 1.29 mmol, 2.5 eq.) and KOAc (152 mg, 1.55 mmol, 3.0 eq.) in DMF (5 mL) was added Pd(dppf)Cl₂—CH₂Cl₂ (37.8 mg, 0.05 mmol, 0.1 eq.) and the reaction mixture was stirred at 80° C. for 2 h under an inert atmosphere. After cooling to room temperature urea hydrogen peroxide adduct (145 mg, 1.55 mmol, 3.0 eq.) was added in portions and the reaction mixture was stirred at room temperature for 1 h. The reaction was quenched by the addition of sat NH₄Cl (50 mL) and extracted with EtOAc (3×50 mL). The combined organic phases were dried over Na₂SO₄ and concentrated in vacuo. Purification by column chromatography (silica gel, 10-20% acetone/CH₂Cl₂) afforded the desired phenol (205 mg, 77%) as a colorless oil. ¹H NMR (700 MHz, DMSO-d₆) δ 9.26 (s, 1H), 8.68 (d, J=7.7 Hz, 1H), 8.32-8.29 (m, 2H), 7.69 (d, J=1.8 Hz, 1H), 7.64-7.46 (m, 5H), 7.42 (t, J=7.7 Hz, 2H), 7.36-7.33 (m, 1H), 7.27 (dt, J=8.0, 2.0 Hz, 1H), 7.08 (dd, J=7.9, 4.4 Hz, 1H), 7.00-6.96 (m, 1H), 6.57-6.55 (m, 1H), 6.55 (d, J=1.4 Hz, 1H), 6.49 (d, J=7.7 Hz, 1H), 6.34 (dd, J=3.6, 1.7 Hz, 1H), 6.32 (s, 1H), 5.54 (dq, J=3.6 Hz, 1H), 4.90 (p, J=7.1 Hz, 1H), 1.35 (d, J=7.0 Hz, 3H). m/z (ESI): 518.2 [M+H⁺]⁺

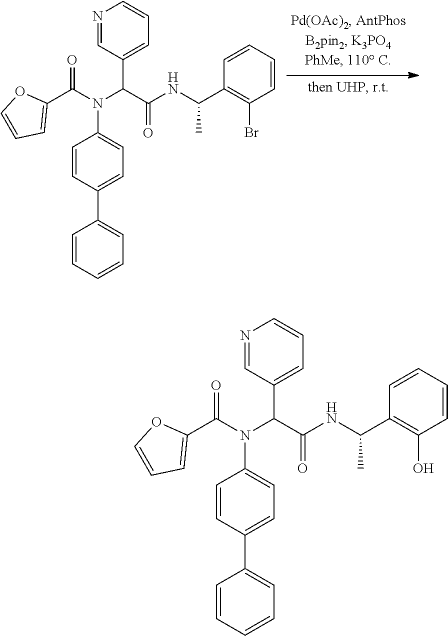 [SMILES: C[C@H](NC(=O)C(c1cccnc1)N(C(=O)c1ccco1)c1ccc(-c2ccccc2)cc1)c1ccccc1Br ; C[C@H](NC(=O)C(c1cccnc1)N(C(=O)c1ccco1)c1ccc(-c2ccccc2)cc1)c1ccccc1O]

The bromide (600 mg, 1.03 mmol, 1.0 eq.), bis(pinacolato)diboron (787 mg, 3.10 mmol, 3.0 eq.) palladium acetate (7.0 mg, 0.03 mmol, 0.03 eq.), AntPhos (25.0 mg, 0.06 mmol, 0.06 eq.) and K₃PO₄ (658 mg, 3.10 mmol, 3.0 eq.) were dissolved in dry toluene (5 mL) and the solution was degassed by bubbling argon through it for 15 min. The reaction mixture was stirred at 110° C. overnight under an inert atmosphere. After cooling to room temperature urea hydrogen peroxide adduct (291 mg, 3.10 mmol, 3.0 eq.) was added in portions and the reaction mixture was stirred at room temperature for 1 h. The reaction was quenched by the addition of sat NH₄Cl (50 mL) and extracted with EtOAc (3×50 mL). The combined organic phases were dried over Na₂SO₄ and concentrated in vacuo. Purification by column chromatography (silica gel, 10-20% acetone/CH₂Cl₂) afforded the desired phenol (130 mg, 24%) as well as the debrominated benzene (103 mg, 20%) as mixtures of diastereomers that weren't separated at this stage. m/z (ESI): 518.2 [M+H⁺]⁺

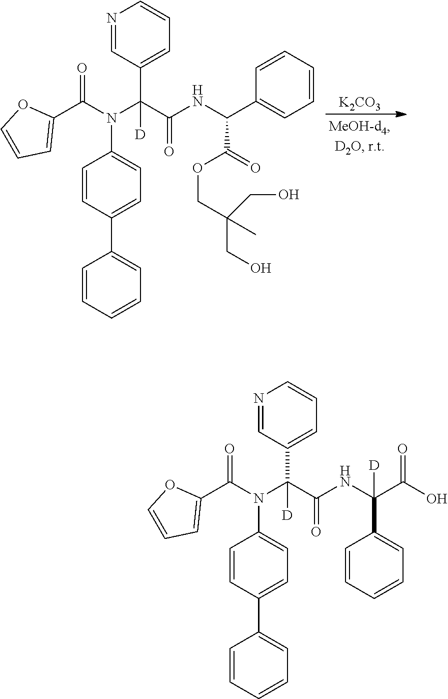

The starting material was obtained according to the previously described procedure by using deuterated 3-pyridinecarboxaldehyde instead of the non-deuterated aldehyde. To a solution of the diol (85.0 mg, 0.13 mmol, 1.0 eq.) in MeOH-d₄ (5 mL) and D₂O (2 mL) was added K₂CO₃ (55-5 mg, 0.40 mmol, 3.0 eq) and the reaction mixture was stirred overnight at room temperature. The mixture was acidified using a solution of conc. HCl in D₂O and extracted with EtOAc (3×15 mL). The combined organic phases were dried over Na₂SO₄ and concentrated in vacuo. The crude product was purified by RP-HPLC (35-95% MeCN/H₂O+0.1% formic acid) to afford the racemic RR-diastereomer of the carboxylic acid (10 mg, 15%). ¹H NMR (500 MHz, DMSO) δ 12.85 (s, 1H), 9.05 (s, 1H), 8.43 (s, 1H), 8.34 (d, J=4.6 Hz, 1H), 7.71-7.66 (m, 1H), 7.65-7.62 (m, 2H), 7.59-7.50 (m, 3H), 7.46-7.23 (m, 10H), 7.18 (dd, J=7.9, 4.8 Hz, 1H), 6.33 (dd, J=3.6, 1.7 Hz, 1H), 5.50 (d, J=3.5 Hz, 1H). m/z (ESI): 534.2 [M+H⁺]⁺

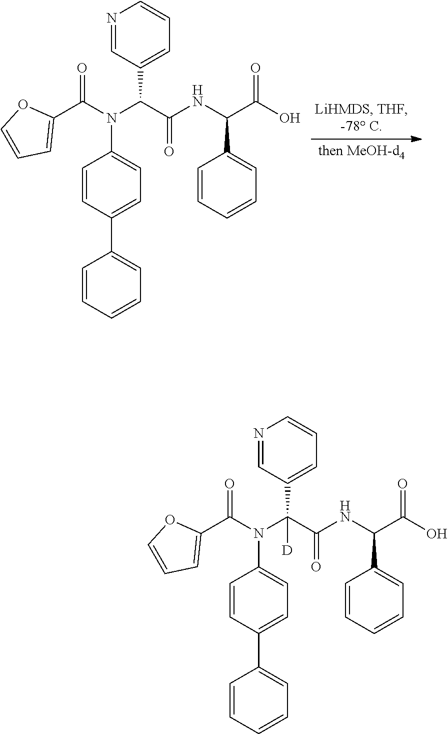 [SMILES: O=C(O)[C@H](NC(=O)[C@@H](c1cccnc1)N(C(=O)c1ccco1)c1ccc(-c2ccccc2)cc1)c1ccccc1 ; [2H][C@@](C(=O)N[C@@H](C(=O)O)c1ccccc1)(c1cccnc1)N(C(=O)c1ccco1)c1ccc(-c2ccccc2)cc1]

To a solution of the carboxylic acid (84.4 mg, 0.16 mmol, 1.0 eq.) in THF was added LiHMDS (1 M in THF, 0.32 mL, 0.32 mmol, 2.0 eq.) dropwise at −78° C. The reaction mixture was stirred at −78° C. for 1 h and then quenched by the addition of MeOH-d₄. The mixture was warmed to room temperature and stirred for an additional 30 min. Sat. NH₄Cl was added and the aqueous phase was extracted with EtOAc (3×15 mL). The combined organic phases were dried over Na₂SO₄ and concentrated in vacuo. The crude product was purified by RP-HPLC (35-95% MeCN/H₂O+0.1% formic acid) to afford the RR-diastereomer of the mono-deuterated carboxylic acid (29.2 mg, 35%). ¹H NMR (500 MHz, DMSO) δ 12.81 (s, 1H), 9.06 (d, J=6.5 Hz, 1H), 8.43 (d, J=2.3 Hz, 1H), 8.34 (dd, J=4.8, 1.7 Hz, 1H), 7.69 (d, J=1.6 Hz, 1H), 7.65-7.62 (m, 2H), 7.60-7.50 (m, 3H), 7.46-7.33 (m, 10H), 7.18 (dd, J=7.9, 4.8 Hz, 1H), 6.34 (dd, J=3.6, 1.7 Hz, 1H), 5.56 (d, J=3.5 Hz, 1H), 5.37-5.29 (m, 1H). m/z (ESI): 533.0 [M+H⁺]⁺

### General Procedures

### General Procedure A

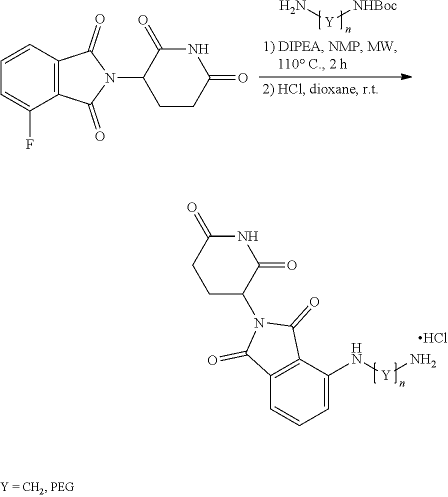

The amine building blocks are synthesized adapting a procedure from the literature [Org. Lett. 2019, 21, 3838]. 4-Fluorothalidomide (1.0 eq.) and the respective amine (1.3 eq.) are dissolved in NMP (0.2-0.5 M) in a microwave tube. DIPEA (3.0 eq.) is added and the tube is sealed. The reaction mixture is stirred under microwave irradiation at 110° C. for 2 h. Afterwards the mixture is diluted with EtOAc and washed with brine. The organic phase is dried over Na₂SO₄ and concentrated in vacuo at 55° C. to remove NMP completely. Purification by flash column chromatography (silica gel) affords the Boc-protected amine intermediate as a bright yellow-green solid. This intermediate is stirred with HCl (4 M, 15 eq.) in dioxane for 1 h at room temperature. The reaction mixture is concentrated in vacuo to afford the ammonium hydrochloride as a bright yellow-green solid.

### General Procedure B

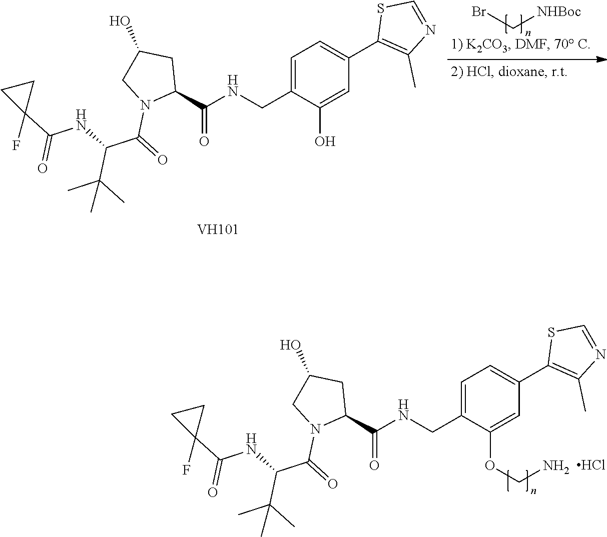

VH101 (1.0 eq.) is dissolved in DMF (0.1-0.2 M) and the respective bromide (1.5 eq.) and K₂CO₃ (2.5 eq.) are added. The reaction mixture is heated at 70° C. until LCMS analysis indicates complete conversion of the starting material. The reaction is quenched with sat. NH₄Cl and extracted with EtOAc (3×). The combined organic phases are washed with brine, dried over Na₂SO₄ and concentrated in vacuo. Purification by flash column chromatography (silica gel) affords the Boc-protected amine intermediate as a white solid. This intermediate is stirred with HCl (4 M, 15 eq.) in dioxane for 1 h at room temperature. The reaction mixture is concentrated in vacuo to afford the ammonium hydrochloride as a white solid.

### General Procedure C

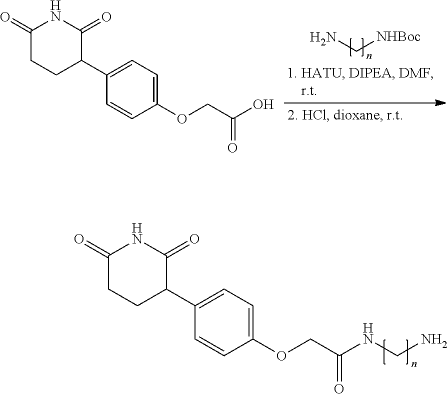

The starting material was synthesized according to the literature [Angew. Chem. Int. Ed. 2021, 60, 26663-26670]. To a solution of the starting piperazine (1.0 eq.), the amine (1.3 eq) and HATU (1.3 eq.) in DMF is added DIPEA (3.0 eq.) and the resulting mixture is stirred overnight at room temperature. The reaction is quenched by the addition of sat. NaHCO₃ and extracted with EtOAc (3×). The combined organic phases are washed with 1M HCl and brine, dried over Na₂SO₄ and concentrated in vacuo. Purification by flash column chromatography (silica gel) affords the Boc-protected amine intermediate. This intermediate is stirred with HCl (4 M, 15 eq.) in dioxane for 1 h at room temperature. The reaction mixture is concentrated in vacuo to afford the ammonium hydrochloride as a colorless solid.

### General Procedure D

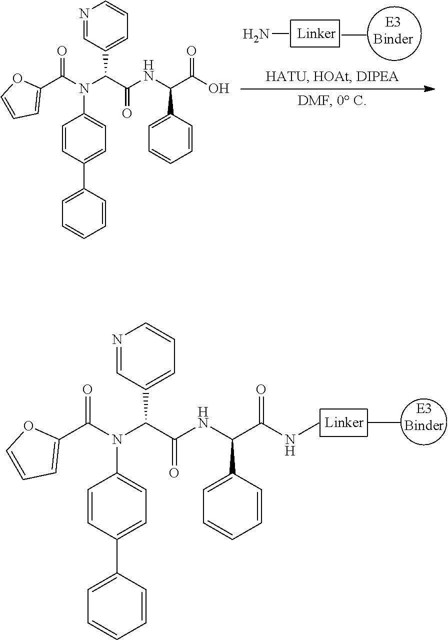

The carboxylic acid (1.0 eq.), the respective amine (1.2 eq.), HATU (1.3 eq.) and HOAt (1.0 eq.) are dissolved in DMF (0.1-0.2 M). DIPEA (3.0 eq.) is added at 0° C. and the reaction mixture is stirred at this temperature until LCMS analysis indicates complete conversion of the starting material. The crude reaction mixture is directly purified by RP-HPLC (35-95% MeCN/H₂O+0.1% formic acid). The product containing fractions are lyophilized to dryness to yield the respective PROTACs.

### General Procedure E

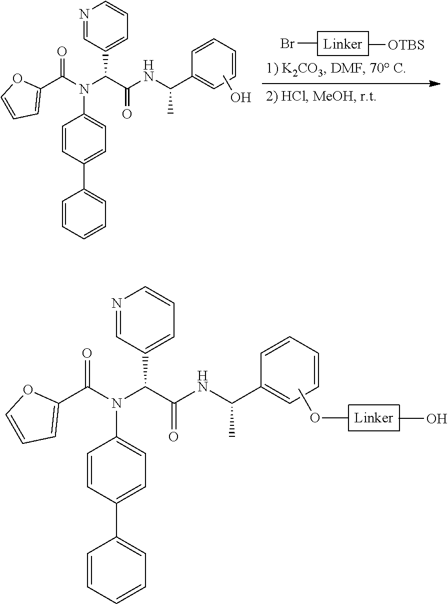

To a solution of the phenol (1.0 eq.) and K₂CO₃ (2.5 eq.) in DMF (0.1-0.2 M) is added the respective bromide (1.8 eq.) and the reaction mixture is stirred at 70° C. overnight. The reaction is quenched by the addition of sat. NH₄Cl and extracted with EtOAc (3×). The combined organic phases are dried over Na₂SO₄ and concentrated in vacuo. Purification by flash column chromatography (silica gel) affords the TBS-protected intermediate. This intermediate is stirred in a mixture of HCl in MeOH (10-15 eq) for 30 min at room temperature. The reaction is quenched by the addition of sat. NaHCO₃ and extracted with EtOAc (3×). The combined organic phases are washed with sat. NH₄Cl and brine, dried over Na₂SO₄, and concentrated in vacuo. The resulting alcohol was used without further purification in the next step.

### General Procedure F

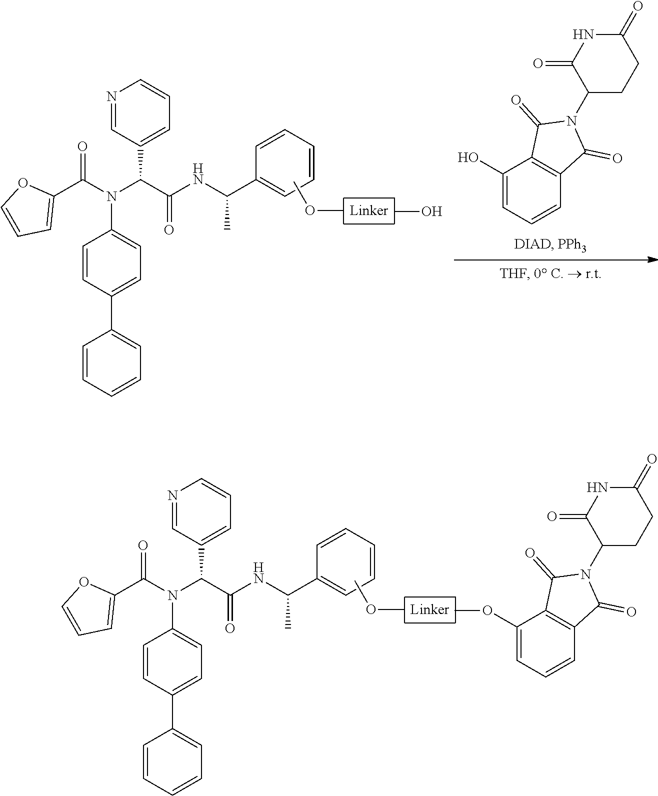

To a solution of the respective alcohol (1.0 eq.), 4-hydroxythalidomide (2.0 eq.) and triphenylphosphine (2.0 eq) in THF is added DIAD (2.0 eq.) dropwise at 0° C. The reaction is stirred at room temperature until LCMS analysis indicates complete conversion of the starting material. The reaction mixture is concentrate in vacuo and directed purified by RP-HPLC (20-95% MeCN/H₂O+0.1% formic acid). The product containing fractions are lyophilized to dryness to yield the respective PROTACs.

TABLE 1

| Cmpd | | |
|---|---|---|
| # | Compound Structure | m/z |
| TS284F1 (rac) | 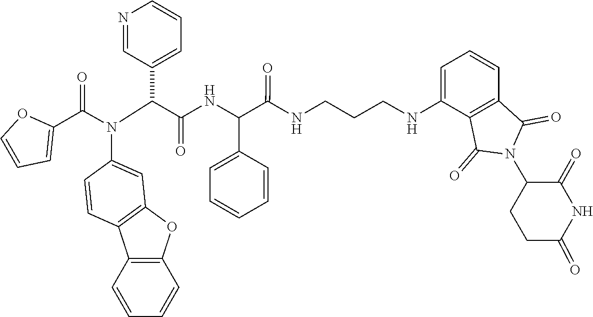 | 857.9 [M + H]⁺ |
| TS296F1 (rac) | 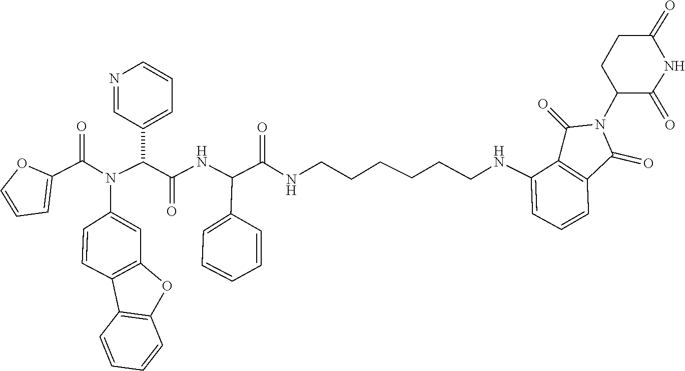 | 899.9 [M + H]⁺ |
| TS297F1 (rac) | 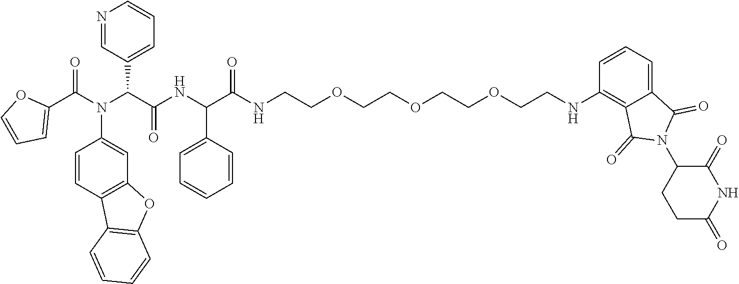 [SMILES: O=C1CCC(N2C(=O)c3cccc(NCCOCCOCCOCCNC(=O)C(NC(=O)[C@@H](c4cccnc4)N(C(=O)c4ccco4)c4ccc5c(c4)oc4ccccc45)c4ccccc4)c3C2=O)C(=O)N1] | 975.9 [M + H]⁺ |
| TS301F1 (rac) | 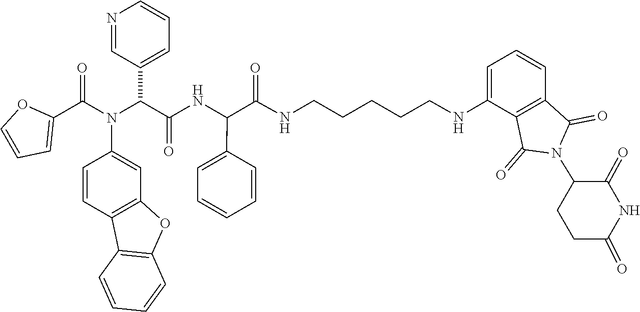 [SMILES: O=C1CCC(N2C(=O)c3cccc(NCCCCCNC(=O)C(NC(=O)[C@@H](c4cccnc4)N(C(=O)c4ccco4)c4ccc5c(c4)oc4ccccc45)c4ccccc4)c3C2=O)C(=O)N1] | 885.9 [M + H]⁺ |
| TS303F1 (rac) | 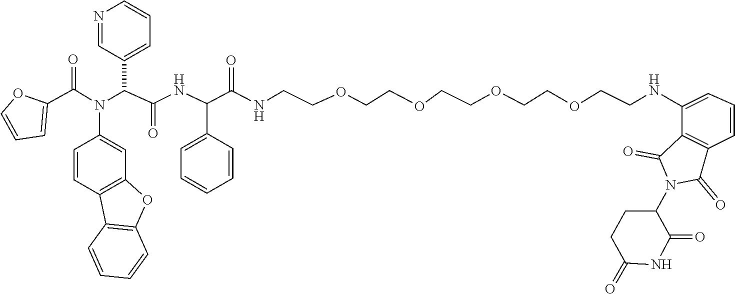 [SMILES: O=C1CCC(N2C(=O)c3cccc(NCCOCCOCCOCCOCCNC(=O)C(NC(=O)[C@@H](c4cccnc4)N(C(=O)c4ccco4)c4ccc5c(c4)oc4ccccc45)c4ccccc4)c3C2=O)C(=O)N1] | 1019.9 [M + H]⁺ |
| TS312F1 (rac) | 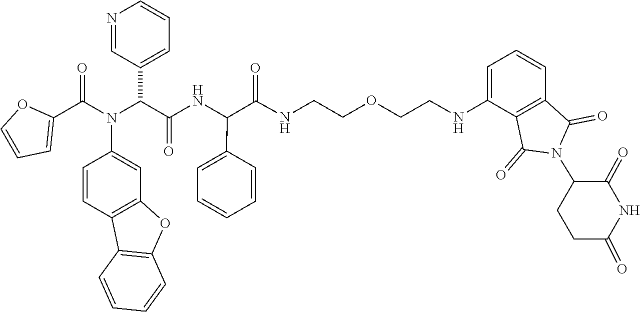 [SMILES: O=C1CCC(N2C(=O)c3cccc(NCCOCCNC(=O)C(NC(=O)[C@@H](c4cccnc4)N(C(=O)c4ccco4)c4ccc5c(c4)oc4ccccc45)c4ccccc4)c3C2=O)C(=O)N1] | 888.3 [M + H]⁺ |
| TS313F1 (rac) | 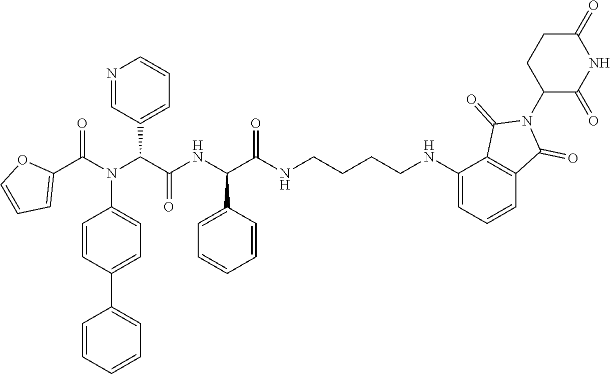 [SMILES: O=C1CCC(N2C(=O)c3cccc(NCCCCNC(=O)[C@H](NC(=O)[C@@H](c4cccnc4)N(C(=O)c4ccco4)c4ccc(-c5ccccc5)cc4)c4ccccc4)c3C2=O)C(=O)N1] | 872.3 [M + H]⁺ |
| TS355F1 | 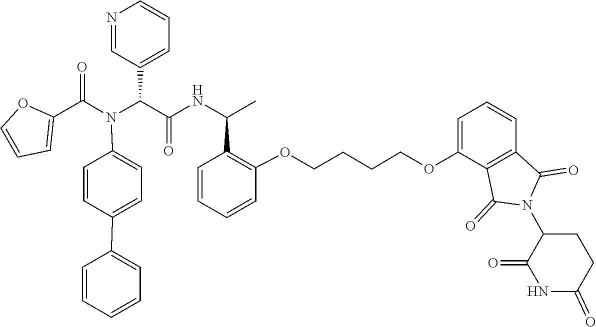 [SMILES: C[C@H](NC(=O)[C@@H](c1cccnc1)N(C(=O)c1ccco1)c1ccc(-c2ccccc2)cc1)c1ccccc1OCCCCOc1cccc2c1C(=O)N(C1CCC(=O)NC1=O)C2=O] | 846.3 [M + H]⁺ |
| TS325F2 | 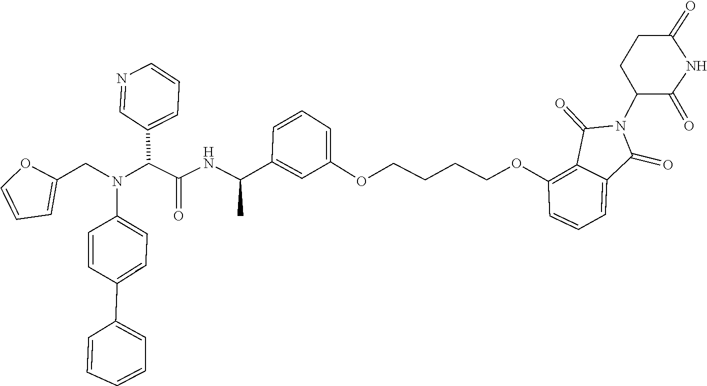 | 846.3 [M + H]⁺ |
| TS335F2 | 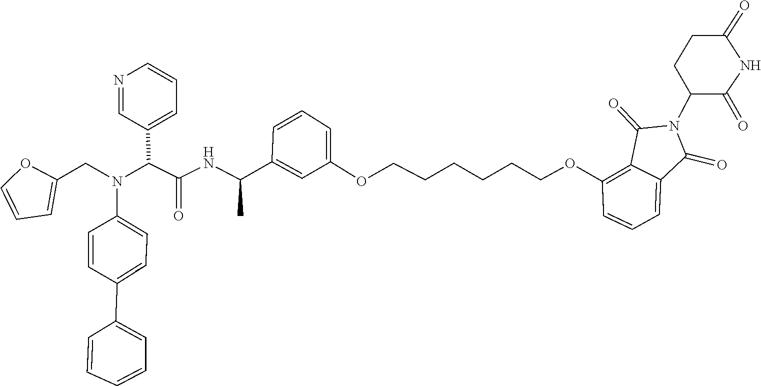 | 874.3 [M + H]⁺ |
| TS338F2 | 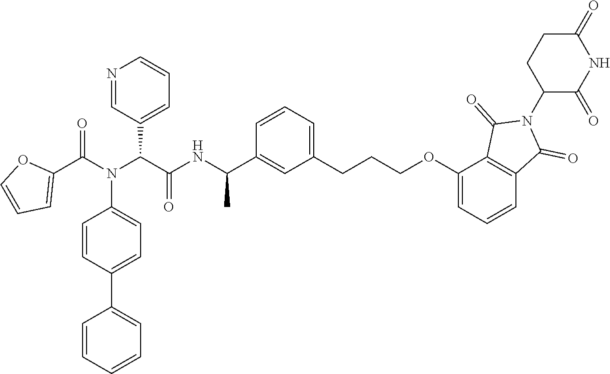 [SMILES: C[C@@H](NC(=O)[C@@H](c1cccnc1)N(C(=O)c1ccco1)c1ccc(-c2ccccc2)cc1)c1cccc(CCCOc2cccc3c2C(=O)N(C2CCC(=O)NC2=O)C3=O)c1] | 818.3 [M + H]⁺ |
| TS339F2 | 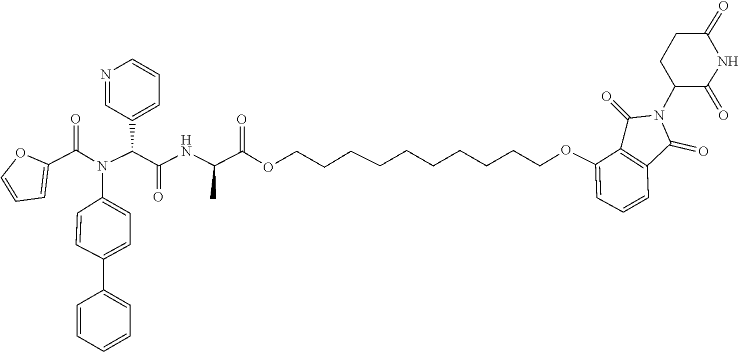 | 902.4 [M + H]⁺ |
| TS366F1 (rac) TS449F1 (80% ee) | 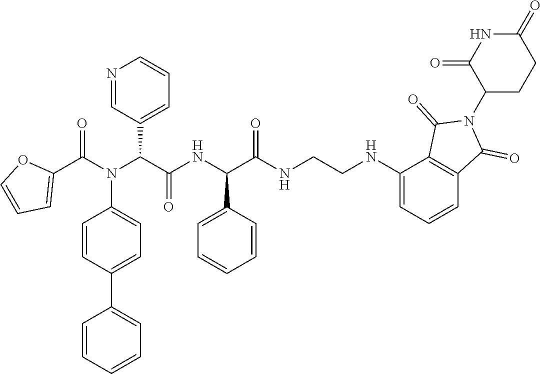 [SMILES: O=C1CCC(N2C(=O)c3cccc(NCCNC(=O)[C@H](NC(=O)[C@@H](c4cccnc4)N(C(=O)c4ccco4)c4ccc(-c5ccccc5)cc4)c4ccccc4)c3C2=O)C(=O)N1] | 830.2 [M + H]⁺ |
| TS361F1 (rac) TS450F1 (80% ee) | 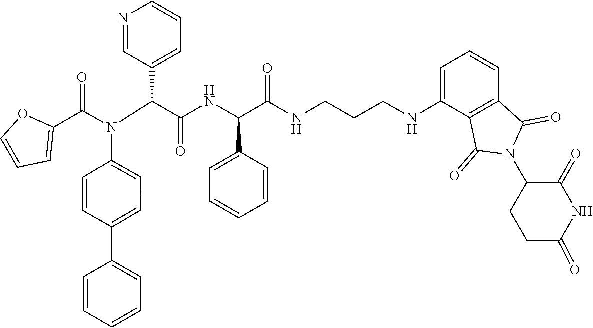 | 844.3 [M + H]⁺ |
| TS365F1 (rac) | 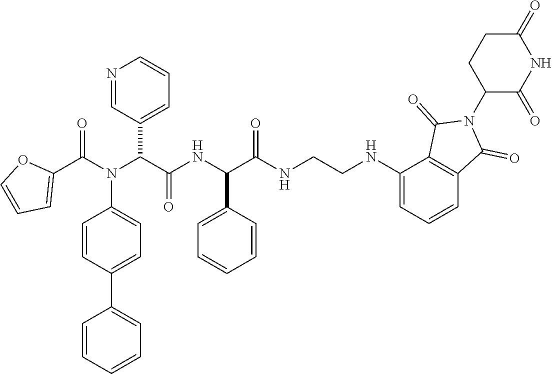 [SMILES: O=C1CCC(N2C(=O)c3cccc(NCCNC(=O)[C@H](NC(=O)[C@@H](c4cccnc4)N(C(=O)c4ccco4)c4ccc(-c5ccccc5)cc4)c4ccccc4)c3C2=O)C(=O)N1] | 844.3 [M + H]⁺ |
| TS369F1 (rac) | 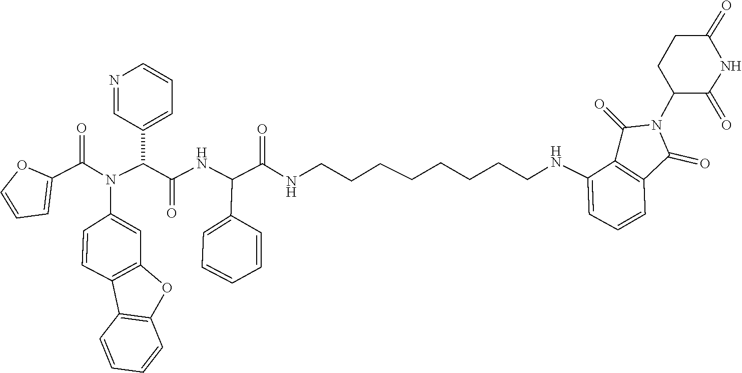 | 928.3 [M + H]⁺ |
| TS370F1 (rac) | 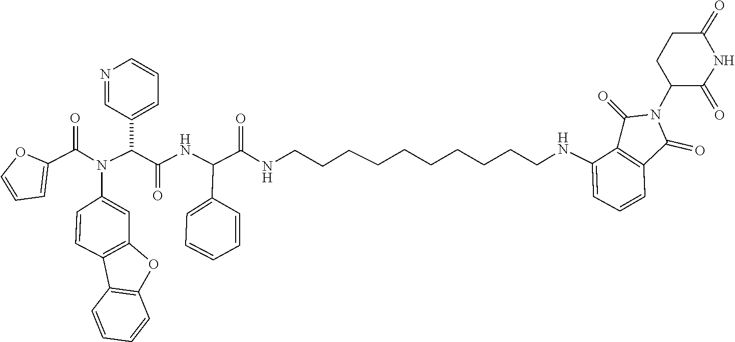 | 956.3 [M + H]⁺ |
| TS360F1 (rac) TS451F1 (80% ee) | 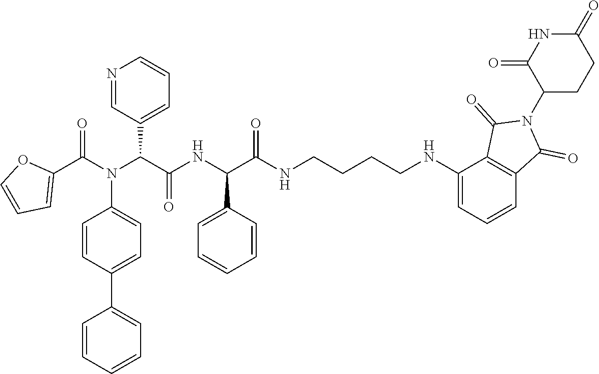 [SMILES: O=C1CCC(N2C(=O)c3cccc(NCCCCNC(=O)[C@H](NC(=O)[C@@H](c4cccnc4)N(C(=O)c4ccco4)c4ccc(-c5ccccc5)cc4)c4ccccc4)c3C2=O)C(=O)N1] | 858.3 [M + H]⁺ |
| TS453F1 (80% ee) | 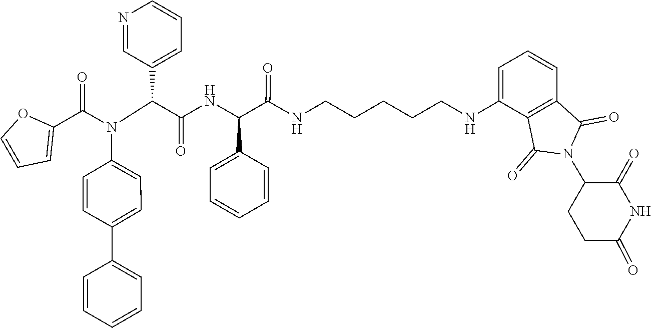 | 874.5 [M + H]⁺ |
| TS454F1 (80% ee) | 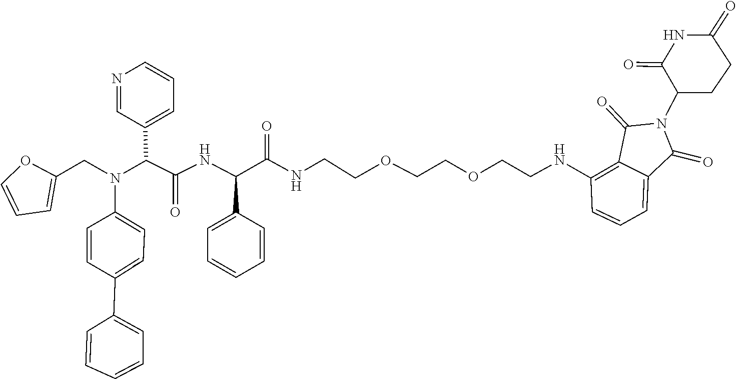 | 918.5 [M + H]⁺ |

TABLE 2

| Cmpd | | |
|---|---|---|
| # | Compound Structure | m/z |
| TS363F1 (rac) TS455F1 (80% ee) | 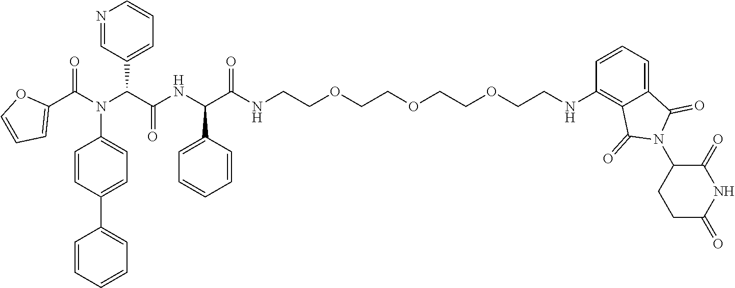 [SMILES: O=C1CCC(N2C(=O)c3cccc(NCCOCCOCCOCCNC(=O)[C@H](NC(=O)[C@@H](c4cccnc4)N(C(=O)c4ccco4)c4ccc(-c5ccccc5)cc4)c4ccccc4)c3C2=O)C(=O)N1] | 962.3 [M + H]⁺ |
| TS362F1 (rac) TS456F1 (80% ee) | 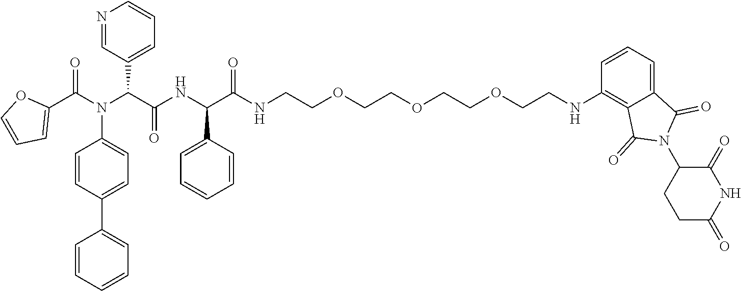 [SMILES: O=C1CCC(N2C(=O)c3cccc(NCCOCCOCCOCCNC(=O)[C@H](NC(=O)[C@@H](c4cccnc4)N(C(=O)c4ccco4)c4ccc(-c5ccccc5)cc4)c4ccccc4)c3C2=O)C(=O)N1] | 1006.3 [M + H]⁺ |
| TS457F1 (rac) | 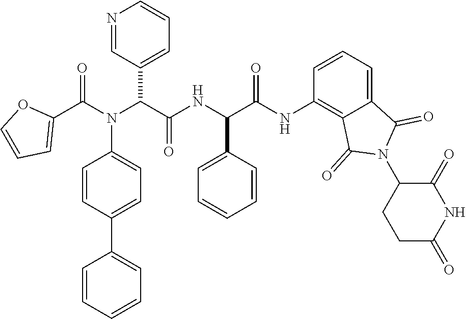 [SMILES: O=C1CCC(N2C(=O)c3cccc(NC(=O)[C@H](NC(=O)[C@@H](c4cccnc4)N(C(=O)c4ccco4)c4ccc(-c5ccccc5)cc4)c4ccccc4)c3C2=O)C(=O)N1] | [M + H]⁺ |
| TS462 (80% ee) | 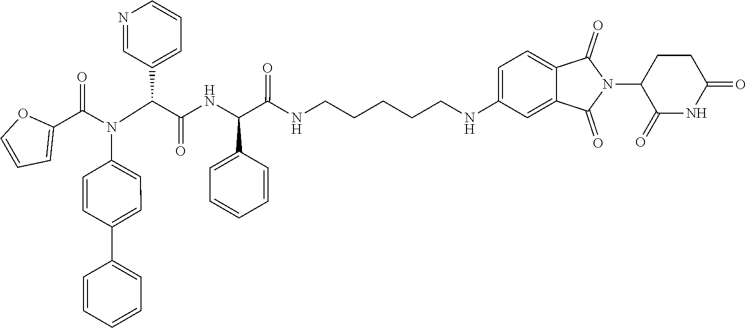 | 874.5 [M + H]⁺ |
| TS464 (80% ee) | 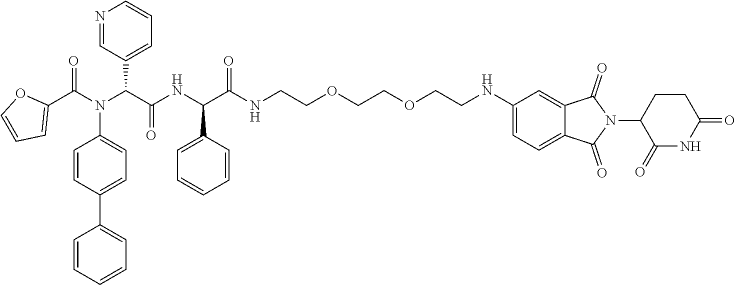 [SMILES: O=C1CCC(N2C(=O)c3ccc(NCCOCCOCCNC(=O)[C@H](NC(=O)[C@@H](c4cccnc4)N(C(=O)c4ccco4)c4ccc(-c5ccccc5)cc4)c4ccccc4)cc3C2=O)C(=O)N1] | 918.6 [M + H]⁺ |
| TS465 (80% ee) | 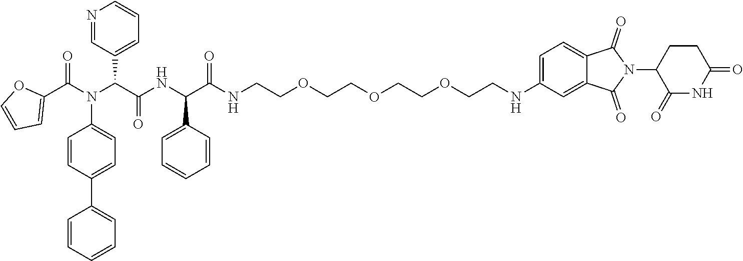 [SMILES: O=C1CCC(N2C(=O)c3ccc(NCCOCCOCCOCCNC(=O)[C@H](NC(=O)[C@@H](c4cccnc4)N(C(=O)c4ccco4)c4ccc(-c5ccccc5)cc4)c4ccccc4)cc3C2=O)C(=O)N1] | 962.6 [M + H]⁺ |
| TS466 (80% ee) | 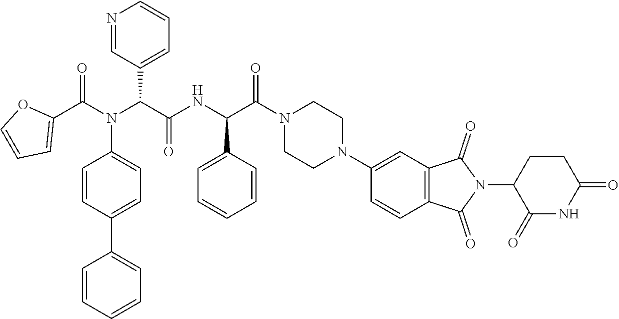 [SMILES: O=C1CCC(N2C(=O)c3ccc(N4CCN(C(=O)[C@H](NC(=O)[C@@H](c5cccnc5)N(C(=O)c5ccco5)c5ccc(-c6ccccc6)cc5)c5ccccc5)CC4)cc3C2=O)C(=O)N1] | 856.5 [M + H]⁺ |
| TS470 (80% ee) | 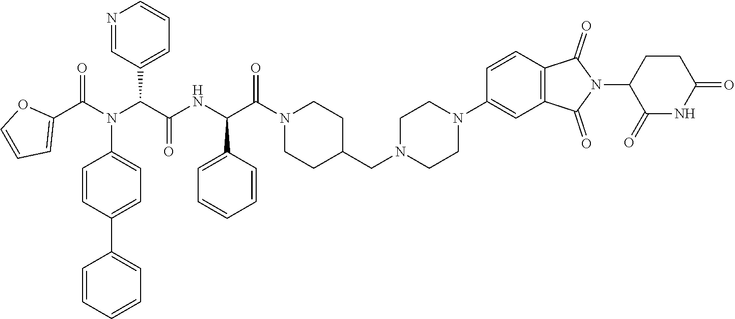 [SMILES: O=C1CCC(N2C(=O)c3ccc(N4CCN(CC5CCN(C(=O)[C@H](NC(=O)[C@@H](c6cccnc6)N(C(=O)c6ccco6)c6ccc(-c7ccccc7)cc6)c6ccccc6)CC5)CC4)cc3C2=O)C(=O)N1] | 953.6 [M + H]⁺ 477.4 [M + 2H]²⁺ |
| TS471 (80% ee) | 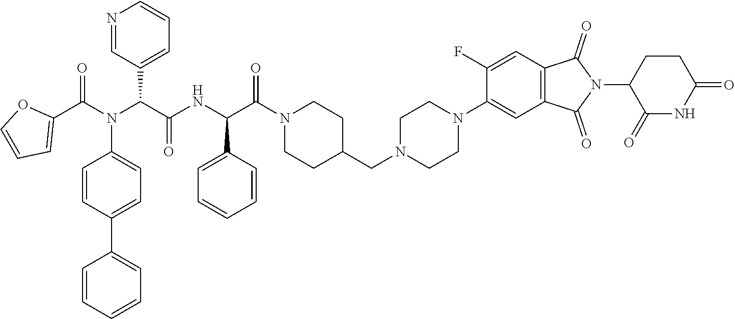 [SMILES: O=C1CCC(N2C(=O)c3cc(F)c(N4CCN(CC5CCN(C(=O)[C@H](NC(=O)[C@@H](c6cccnc6)N(C(=O)c6ccco6)c6ccc(-c7ccccc7)cc6)c6ccccc6)CC5)CC4)cc3C2=O)C(=O)N1] | 971.5 [M + H]⁺ 486.4 [M + 2H]²⁺ |
| TS476 (80% ee) | 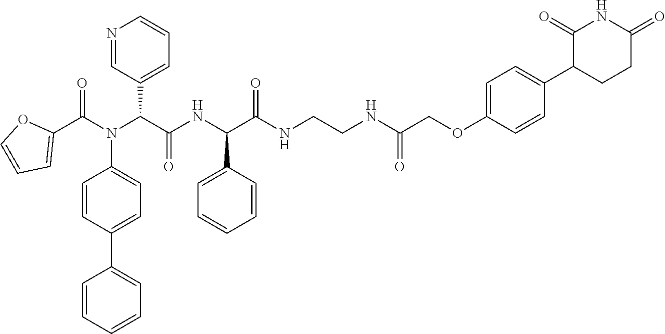 [SMILES: O=C(COc1ccc(C2CCC(=O)NC2=O)cc1)NCCNC(=O)[C@H](NC(=O)[C@@H](c1cccnc1)N(C(=O)c1ccco1)c1ccc(-c2ccccc2)cc1)c1ccccc1] | 819.5 [M + H]⁺ |
| TS477 (80% ee) | 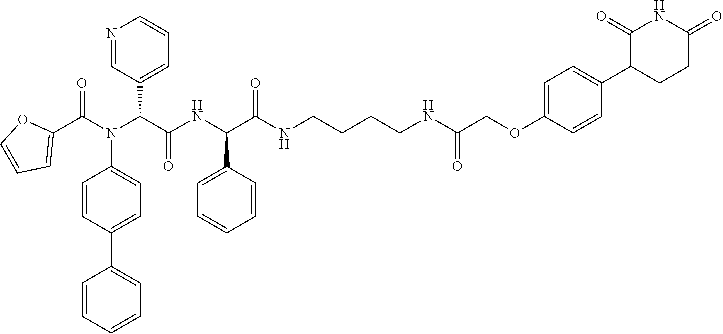 [SMILES: O=C(COc1ccc(C2CCC(=O)NC2=O)cc1)NCCCCNC(=O)[C@H](NC(=O)[C@@H](c1cccnc1)N(C(=O)c1ccco1)c1ccc(-c2ccccc2)cc1)c1ccccc1] | 847.5 [M + H]⁺ |
| TS524 (rac) | 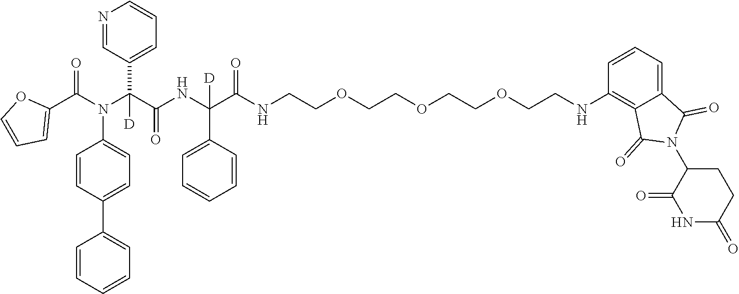 [SMILES: [2H]C(NC(=O)[C@]([2H])(c1cccnc1)N(C(=O)c1ccco1)c1ccc(-c2ccccc2)cc1)(C(=O)NCCOCCOCCOCCNc1cccc2c1C(=O)N(C1CCC(=O)NC1=O)C2=O)c1ccccc1] | 964.2 [M + H]⁺ |
| TS577 (80% ee) | 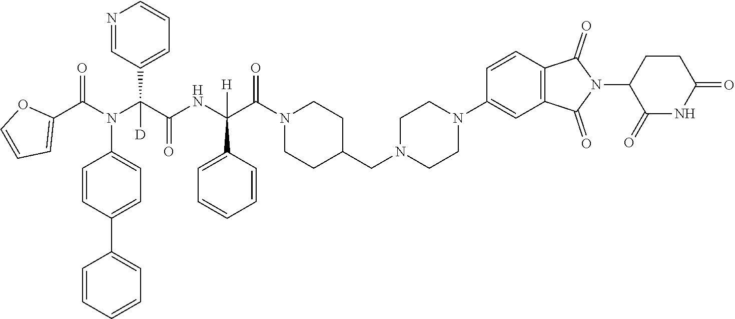 [SMILES: [2H][C@@](C(=O)N[C@H](C(=O)N1CCC(CN2CCN(c3ccc4c(c3)C(=O)N(C3CCC(=O)NC3=O)C4=O)CC2)CC1)c1ccccc1)(c1cccnc1)N(C(=O)c1ccco1)c1ccc(-c2ccccc2)cc1] | 954.1 [M + H]⁺ 477.7 [M + 2H]²⁺ |
| TS578 (80% ee) | 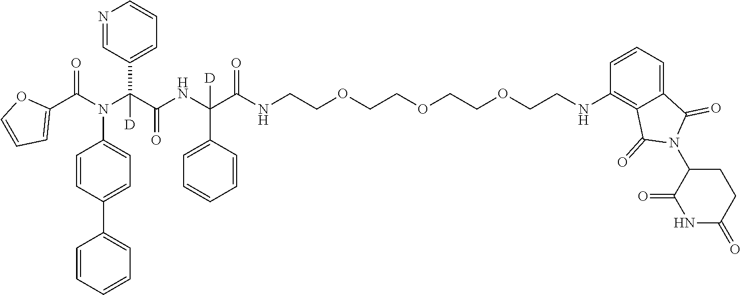 | 963.0 [M + H]⁺ |
| TS467 (80% ee) | 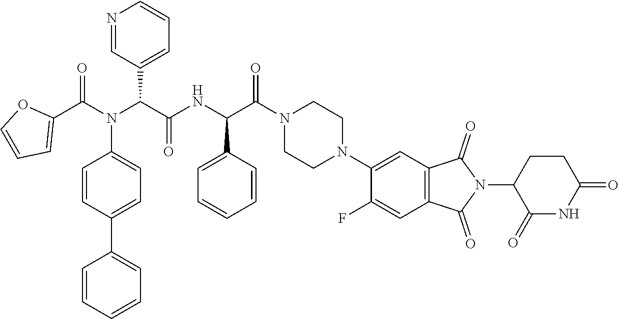 [SMILES: O=C1CCC(N2C(=O)c3cc(F)c(N4CCN(C(=O)[C@H](NC(=O)[C@@H](c5cccnc5)N(C(=O)c5ccco5)c5ccc(-c6ccccc6)cc5)c5ccccc5)CC4)cc3C2=O)C(=O)N1] | 874.5 [M + H]⁺ |

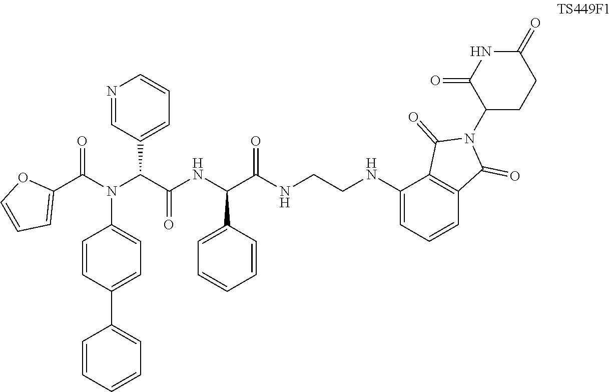 [SMILES: O=C1CCC(N2C(=O)c3cccc(NCCNC(=O)[C@H](NC(=O)[C@@H](c4cccnc4)N(C(=O)c4ccco4)c4ccc(-c5ccccc5)cc4)c4ccccc4)c3C2=O)C(=O)N1]

¹H NMR (700 MHz, DMSO-d₆): δ=11.10 (s, 1H), 8.98 (d, J=7.4 Hz, 1H), 8.53-8.48 (m, 2H), 8.42 (d, J=4.9 Hz, 1H), 7.71-7.66 (m, 2H), 7.64-7.59 (m, 2H), 7.59-7.51 (m, 2H), 7.48 (ddd, J=8.9, 7.1, 2.2 Hz, 1H), 7.46-7.41 (m, 4H), 7.40-7.18 (m, 7H), 7.10 (d, J=8.7 Hz, 1H), 7.00 (dd, J=7.0, 1.7 Hz, 1H), 6.67 (t, J=6.3 Hz, 1H), 6.54 (s, 1H), 6.33 (dd, J=3.6, 1.7 Hz, 1H), 5.54 (d, J=3.6 Hz, 1H), 5.46 (dd, J=7.3, 5.5 Hz, 1H), 5.04 (dd, J=12.8, 5.4 Hz, 1H), 3.36-3.16 (m, 5H), 2.90-2.83 (m, 1H), 2.61-2.55 (m, 1H), 1.99 (ddt, J=13.6, 8.2, 5.9 Hz, 1H) ppm.

¹³C NMR (176 MHz, DMSO-d₆): δ 173.3, 171.9, 170.6, 170.5, 169.1, 168.8, 167.8, 158.7, 146.7, 146.6, 146.6, 145.9, 140.0, 139.0, 139.0, 138.3, 136.7, 132.6, 132.0, 129.5, 128.9, 128.8, 128.5, 128.4, 128.1, 127.7, 127.7, 127.1, 127.0, 117.5, 116.9, 111.9, 111.1, 109.7, 109.7, 62.3, 57.4, 57.4, 49.0, 38.7, 31.4, 22.6 ppm.

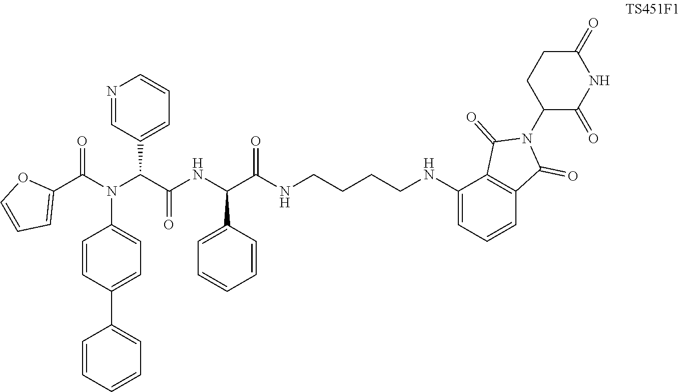 [SMILES: O=C1CCC(N2C(=O)c3cccc(NCCCCNC(=O)[C@H](NC(=O)[C@@H](c4cccnc4)N(C(=O)c4ccco4)c4ccc(-c5ccccc5)cc4)c4ccccc4)c3C2=O)C(=O)N1]

¹H NMR (700 MHz, DMSO-d₆): δ=11.09 (s, 1H), 8.93 (d, J=7.3 Hz, 1H), 8.49 (s, 1H), 8.41 (s, 1H), 8.27 (t, J=5.7 Hz, 1H), 7.71-7.65 (m, 2H), 7.63-7.59 (m, 2H), 7.59-7.49 (m, 3H), 7.47-7.39 (m, 4H), 7.38-7.15 (m, 7H), 7.04 (d, J=8.6 Hz, 1H), 7.00 (d, J=7.0 Hz, 1H), 6.53 (s, 1H), 6.47 (s, 1H), 6.33 (dd, J=3.7, 1.7 Hz, 1H), 5.54 (d, J=3.6 Hz, 1H), 5.46 (d, J=7.3 Hz, 1H), 5.03 (dd, J=12.9, 5.5 Hz, 1H), 3.25-3.19 (m, 2H), 3.12-3.00 (m, 2H), 2.86 (dddd, J=17.2, 14.0, 5.0, 1.3 Hz, 1H), 2.62-2.56 (m, 1H), 2.54-2.46 (m, 1H), 2.03-1.98 (m, 1H), 1.48-1.38 (m, 4H) ppm.

¹³C NMR (126 MHz, DMSO-d₆): δ 173.3, 170.6, 169.9, 169.4, 168.9, 167.8, 158.7, 151.0, 148.5, 146.8, 146.7, 145.9, 139.9, 139.3, 139.0, 138.9, 138.7, 136.8, 132.6, 132.1, 131.6, 129.5, 128.7, 128.4, 128.1, 127.6, 127.0, 123.8, 117.7, 116.8, 111.9, 110.9, 109.4, 62.4, 57.4, 49.0, 41.9, 38.7, 31.4, 26.7, 26.4, 22.6 ppm.

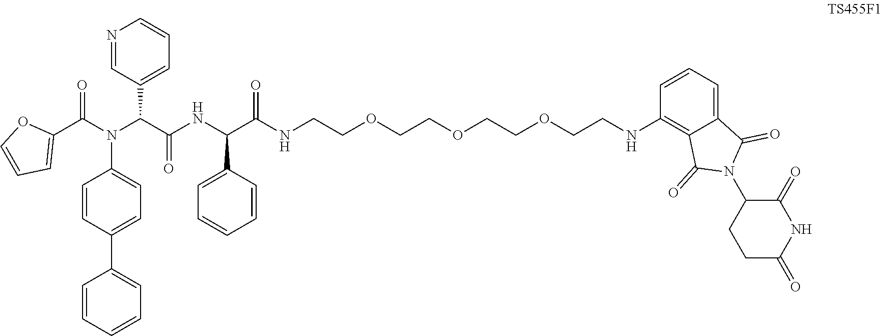 [SMILES: O=C1CCC(N2C(=O)c3cccc(NCCOCCOCCOCCNC(=O)[C@H](NC(=O)[C@@H](c4cccnc4)N(C(=O)c4ccco4)c4ccc(-c5ccccc5)cc4)c4ccccc4)c3C2=O)C(=O)N1]

¹H NMR (700 MHz, DMSO-d₆): δ=11.09 (s, 1H), 8.91 (d, J=7.6 Hz, 1H), 8.42 (s, 1H), 8.35 (d, J=5.0 Hz, 1H), 8.34 (t, J=5.8 Hz, 1H), 7.68 (d, J=1.6 Hz, 1H), 7.64-7.61 (m, 2H), 7.56 (dd, J=8.6, 7.1 Hz, 1H), 7.58-7.50 (m, 4H), 7.47-7.41 (m, 4H), 7.35 (td, J=7.5, 1.9 Hz, 4H), 7.30-7.28 (m, 1H), 7.21 (dd, J=7.9, 4.9 Hz, 1H), 7.12 (d, J=8.6 Hz, 1H), 7.03 (d, J=7.0 Hz, 1H), 6.59 (t, J=5.8 Hz, 1H), 6.53 (s, 1H), 6.33 (dd, J=3.7, 1.7 Hz, 1H), 5.54 (d, J=7.5 Hz, 1H), 5.52 (d, J=3.6 Hz, 1H), 5.05 (dd, J=12.9, 5.5 Hz, 1H), 3.58 (t, J=5.5 Hz, 2H), 3.53-3.51 (m, 2H), 3.48 (dd, J=5.8, 3.5 Hz, 2H), 3.44 (t, J=5.3 Hz, 4H), 3.41 (td, J=4.8, 3.4 Hz, 4H), 3.20-3.11 (m, 2H), 2.87 (ddd, J=17.1, 13.9, 5.5 Hz, 1H), 2.60-2.50 (m, 2H), 2.04-1.98 (m, 1H) ppm.

¹³C NMR (176 MHz, DMSO-d₆): δ 173.3, 170.6, 170.0, 169.4, 169.0, 167.8, 158.7, 146.9, 146.7, 145.9, 140.0, 139.1, 139.0, 138.7, 136.7, 132.5, 132.1, 129.4, 128.7, 128.4, 128.0, 127.7, 127.0, 123.7, 117.9, 116.8, 111.9, 111.2, 109.7, 70.2, 70.2, 70.2, 70.0, 69.3, 69.2, 62.4, 57.2, 49.0, 42.1, 39.3, 31.4, 22.6 ppm.

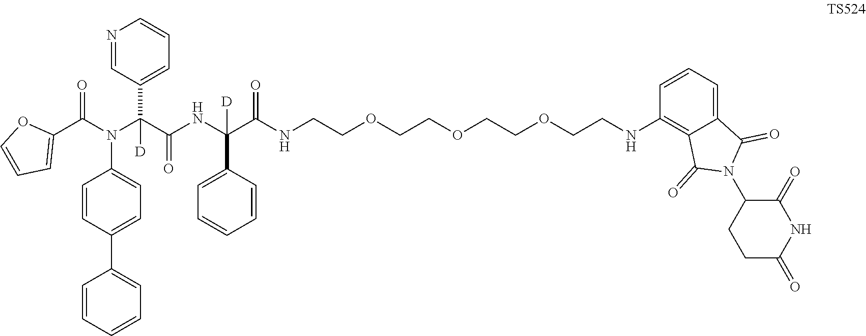 [SMILES: [2H][C@@](NC(=O)[C@]([2H])(c1cccnc1)N(C(=O)c1ccco1)c1ccc(-c2ccccc2)cc1)(C(=O)NCCOCCOCCOCCNc1cccc2c1C(=O)N(C1CCC(=O)NC1=O)C2=O)c1ccccc1]

¹H NMR (700 MHz, DMSO-d₆): δ=11.09 (s, 1H), 8.91 (s, 1H), 8.45 (s, 1H), 8.39 (s, 1H), 8.34 (t, J=5.7 Hz, 1H), 7.68-7.67 (m, 1H), 7.64-7.61 (m, 2H), 7.56 (td, J=10.9, 9.8, 7.3 Hz, 4H), 7.49-7.40 (m, 5H), 7.37-7.33 (m, 3H), 7.31-7.18 (m, 3H), 7.12 (d, J=8.6 Hz, 1H), 7.03 (d, J=7.0 Hz, 1H), 6.59 (t, J=5.9 Hz, 1H), 6.33 (dd, J=3.6, 1.7 Hz, 1H), 5.53 (d, J=3.6 Hz, 1H), 5.05 (dd, J=12.9, 5.4 Hz, 1H), 3.58 (t, J=5.5 Hz, 2H), 3.54-3.51 (m, 2H), 3.49-3.47 (m, 2H), 3.46-3.29 (m, 9H), 3.20-3.11 (m, 2H), 2.87 (ddd, J=17.1, 13.9, 5.5 Hz, 1H), 2.60-2.55 (m, 1H), 2.01 (dddd, J=12.8, 9.5, 4.7, 2.1 Hz, 1H) ppm.

¹³C NMR (176 MHz, DMSO-d₆): δ=173.3, 170.5, 170.0, 169.4, 168.9, 167.8, 158.7, 146.9, 146.7, 145.9, 140.0, 139.1, 139.0, 138.7, 136.7, 132.6, 132.1, 129.4, 128.7, 128.3, 128.0, 127.7, 127.0, 123.8, 117.9, 116.8, 111.8, 111.2, 109.7, 70.2, 70.2, 70.2, 70.0, 69.3, 69.2, 62.1, 56.8, 49.0, 42.1, 39.2, 31.4, 22.6 ppm.

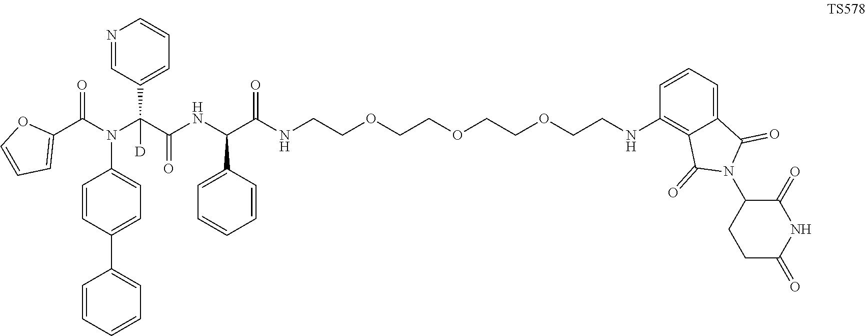 [SMILES: [2H][C@@](C(=O)N[C@@H](C(=O)NCCOCCOCCOCCNc1cccc2c1C(=O)N(C1CCC(=O)NC1=O)C2=O)c1ccccc1)(c1cccnc1)N(C(=O)c1ccco1)c1ccc(-c2ccccc2)cc1]

¹H NMR (700 MHz, DMSO-d₆): δ=11.09 (s, 1H), 8.92 (d, J=7.5 Hz, 1H), 8.46 (s, 1H), 8.39 (s, 1H), 8.34 (t, J=5.7 Hz, 1H), 7.68 (dd, J=1.6, 0.7 Hz, 1H), 7.64-7.61 (m, 2H), 7.59 (d, J=7.8 Hz, 1H), 7.56 (dd, J=8.6, 7.1 Hz, 2H), 7.52 (d, J=6.6 Hz, 2H), 7.47-7.41 (m, 4H), 7.37-7.33 (m, 3H), 7.31-7.22 (m, 3H), 7.12 (d, J=8.6 Hz, 1H), 7.03 (d, J=7.0 Hz, 1H), 6.59 (t, J=5.9 Hz, 1H), 6.33 (dd, J=3.6, 1.7 Hz, 1H), 5.55-5.52 (m, 2H), 5.05 (dd, J=12.9, 5.5 Hz, 1H), 3.58 (t, J=5.5 Hz, 2H), 3.52 (dd, J=5.9, 3.5 Hz, 2H), 3.48 (dd, J=5.8, 3.5 Hz, 2H), 3.46-3.29 (m, 9H), 3.21-3.11 (m, 2H), 2.87 (ddd, J=17.1, 13.9, 5.4 Hz, 1H), 2.58 (dt, J=17.2, 3.1 Hz, 1H), 2.03-1.98 (m, 1H) ppm.

¹³C NMR (176 MHz, DMSO-d₆): δ=173.3, 170.5, 170.0, 169.4, 168.9, 167.8, 158.7, 151.0, 148.5, 146.9, 146.7, 145.9, 140.0, 139.1, 139.0, 138.7, 136.7, 132.6, 132.1, 131.6, 129.4, 128.7, 128.3, 128.0, 127.7, 127.0, 127.0, 123.8, 117.9, 116.8, 111.8, 111.2, 109.7, 70.2, 70.2, 70.2, 70.0, 69.3, 69.2, 62.1, 57.1, 49.0, 42.1, 39.3, 31.4, 22.6 ppm.

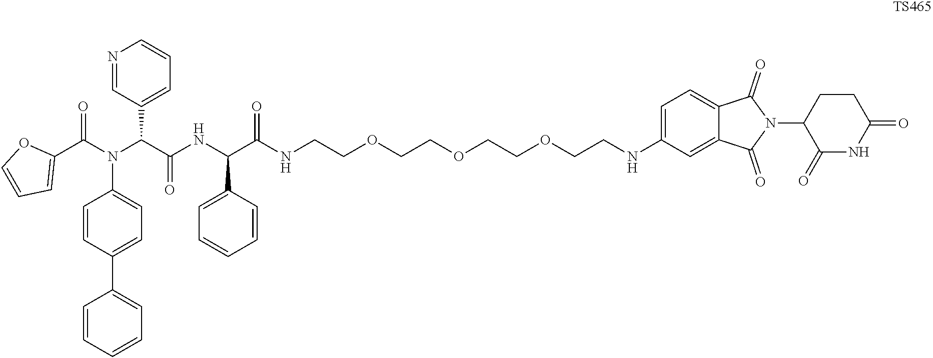 [SMILES: "TS465" ; O=C1CCC(N2C(=O)c3ccc(NCCOCCOCCOCCNC(=O)[C@H](NC(=O)[C@@H](c4cccnc4)N(C(=O)c4ccco4)c4ccc(-c5ccccc5)cc4)c4ccccc4)cc3C2=O)C(=O)N1]

¹H NMR (700 MHz, DMSO-d₆): δ=11.06 (s, 1H), 8.92 (d, J=7.5 Hz, 1H), 8.47 (s, 1H), 8.41 (s, 1H), 8.34 (t, J=5.7 Hz, 1H), 7.68 (dd, J=1.7, 0.7 Hz, 1H), 7.64-7.58 (m, 4H), 7.58-7.49 (m, 3H), 7.48-7.40 (m, 4H), 7.35 (td, J=7.4, 1.6 Hz, 3H), 7.32-7.06 (m, 3H), 6.99 (d, J=2.1 Hz, 1H), 6.88 (dd, J=8.4, 2.2 Hz, 1H), 6.53 (s, 1H), 6.33 (dd, J=3.6, 1.7 Hz, 1H), 5.54 (d, J=4.0 Hz, 2H), 5.53 (d, J=7.7 Hz, 1H), 5.03 (dd, J=12.9, 5.5 Hz, 1H), 3.56 (t, J=5.5 Hz, 2H), 3.52-3.50 (m, 2H), 3.50-3.47 (m, 2H), 3.45-3.29 (m, 8H), 3.21-3.12 (m, 2H), 2.86 (ddd, J=17.1, 13.9, 5.5 Hz, 1H), 2.60-2.47 (m, 2H), 2.01-1.96 (m, 1H) ppm.

¹³C NMR (176 MHz, DMSO-d₆): δ=173.3, 170.7, 170.0, 168.9, 168.2, 167.6, 158.7, 154.9, 150.9, 148.3, 146.7, 145.9, 140.0, 139.5, 139.1, 139.0, 138.6, 134.6, 132.1, 131.8, 129.4, 128.7, 128.6, 128.4, 128.0, 127.7, 127.0, 127.0, 125.5, 123.9, 116.8, 116.6, 111.9, 70.2, 70.2, 70.2, 70.0, 69.2, 69.2, 62.4, 57.2, 49.1, 42.9, 39.3, 31.5, 22.7 ppm.

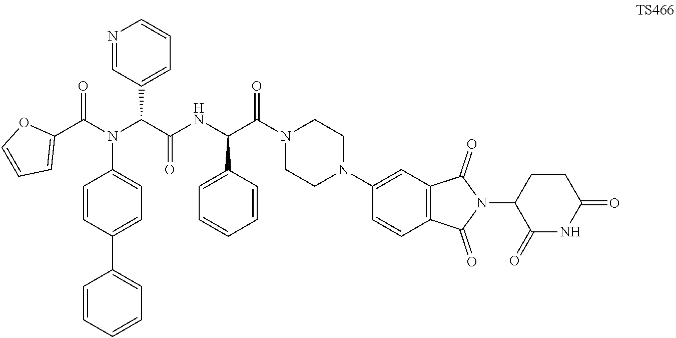 [SMILES: O=C1CCC(N2C(=O)c3ccc(N4CCN(C(=O)[C@H](NC(=O)[C@@H](c5cccnc5)N(C(=O)c5ccco5)c5ccc(-c6ccccc6)cc5)c5ccccc5)CC4)cc3C2=O)C(=O)N1]

¹H NMR (500 MHz, DMSO-d₆): δ=11.09 (s, 1H), 9.03 (d, J=6.8 Hz, 1H), 8.47 (d, J=2.3 Hz, 1H), 8.38 (dd, J=4.9, 1.6 Hz, 1H), 7.70-7.60 (m, 5H), 7.55 (d, J=8.0 Hz, 2H), 7.49-7.31 (m, 9H), 7.30-7.19 (m, 3H), 7.16 (dd, J=8.7, 2.3 Hz, 1H), 6.48 (s, 1H), 6.33 (dd, J=3.6, 1.7 Hz, 1H), 6.00 (d, J=6.8 Hz, 1H), 5.52 (d, J=3.6 Hz, 1H), 5.06 (dd, J=12.8, 5-4 Hz, 1H), 3.70-3.26 (m, 8H), 2.99-2.92 (m, 1H), 2.87 (ddd, J=16.8, 13.8, 5.4 Hz, 1H), 2.62-2.46 (m, 1H), 2.04-1.97 (m, 1H) ppm.

¹³C NMR (126 MHz, DMSO-d₆): δ=173.3, 170.6, 168.8, 168.5, 167.9, 167.4, 158.7, 155.2, 151.3, 148.6, 146.7, 145.9, 139.9, 139.4, 139.0, 139.0, 137.2, 134.2, 132.1, 131.4, 129.5, 129.1, 128.7, 128.5, 128.4, 127.0, 125.4, 123.7, 119.1, 118.3, 116.8, 111.9, 108.5, 62.3, 54.4, 49.2, 44.6, 41.7, 31.4, 22.6 ppm.

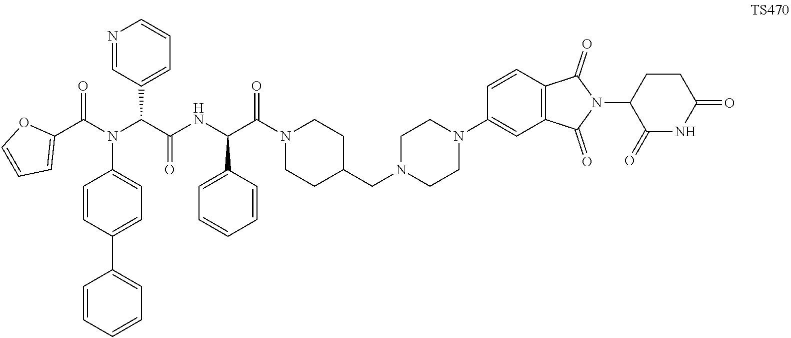 [SMILES: O=C1CCC(N2C(=O)c3ccc(N4CCN(CC5CCN(C(=O)[C@H](NC(=O)[C@@H](c6cccnc6)N(C(=O)c6ccco6)c6ccc(-c7ccccc7)cc6)c6ccccc6)CC5)CC4)cc3C2=O)C(=O)N1]

¹H NMR (500 MHz, DMSO-d₆): δ=11.10 (s, 1H), 8.98-8.80 (m, 1H), 8.40 (d, J=2.4 Hz, 1H), 8.33 (dt, J=4.9, 2.7 Hz, 1H), 7.74-7.12 (m, 22H), 6.46 (d, J=3.0 Hz, 1H), 6.33 (dd, J=3.6, 1.7 Hz, 1H), 5.99-5.87 (m, 1H), 5.50 (d, J=3.6 Hz, 1H), 5.06 (dd, J=12.6, 5.1 Hz, 1H), 4.41-4.22 (m, 1H), 3.96-3.84 (m, 1H), 3.53-3.19 (m, 6H), 3.07-2.71 (m, 2H), 2.62-2.38 (m, 5H), 2.16-1.89 (m, 3H), 1.78-1.35 (m, 3H) ppm.

¹³C NMR (126 MHz, DMSO-d₆): δ 171.2, 168.4, 166.7, 166.5, 166.1, 165.8, 165.6, 165.3, 156.6, 153.0, 149.9, 147.0, 144.6, 143.8, 137.8, 137.0, 136.9, 136.4, 135.4, 132.1, 130.0, 129.0, 127.3, 127.1, 126.9, 126.6, 126.4, 126.3, 126.2, 124.9, 123.3, 121.2, 116.6, 114.7, 109.7, 106.7, 60.4, 60.3, 52.4, 51.9, 47.1, 43.1, 42.9, 39.9, 29.3, 27.9, 20.5 ppm.

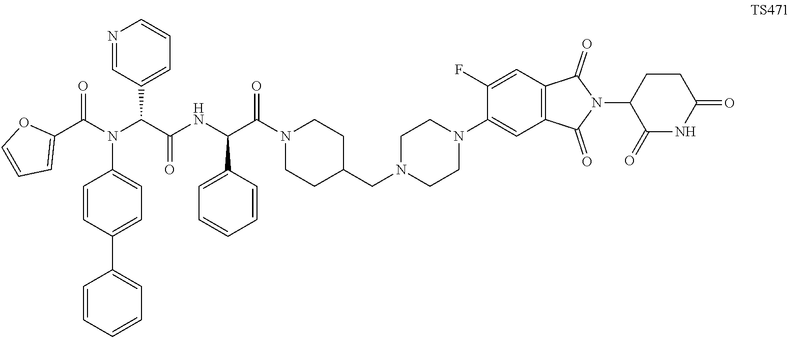 [SMILES: O=C1CCC(N2C(=O)c3cc(F)c(N4CCN(CC5CCN(C(=O)[C@H](NC(=O)[C@@H](c6cccnc6)N(C(=O)c6ccco6)c6ccc(-c7ccccc7)cc6)c6ccccc6)CC5)CC4)cc3C2=O)C(=O)N1]

¹H NMR (500 MHz, DMSO-d₆): δ=11.12 (s, 1H), 9.00-8.77 (m, 1H), 8.40 (d, J=2.4 Hz, 1H), 8.34 (t, J=5.2 Hz, 1H), 7.81-7.12 (m, 20H), 6.46 (d, J=3.1 Hz, 1H), 6.33 (dd, J=3.5, 1.7 Hz, 1H), 6.00-5.88 (m, 1H), 5.50 (d, J=3.6 Hz, 1H), 5.10 (dd, J=13.0, 5.4 Hz, 1H), 4.43-4.21 (m, 1H), 3.94-3.85 (m, 1H), 3.35-3.10 (m, 6H), 3.03-2.72 (m, 2H), 2.50 (p, J=1.9 Hz, 6H), 2.21-1.89 (m, 3H), 1.77-1.61 (m, 2H), 1.59-1.35 (m, 1H) ppm.

¹³C NMR (176 MHz, DMSO-d₆): δ=173.2, 170.4, 168.8, 168.6, 167.7, 167.1, 166.6, 158.7, 158.4, 157.8 (d, J=253.6 Hz), 152.0, 151.9, 149.2, 149.1, 146.8, 145.9, 139.9, 139.9, 139.1, 139.0, 138.5, 137.6, 132.1, 131.1, 131.0, 129.4, 129.2, 129.0, 128.7, 128.5, 128.4, 128.3, 123.3, 116.8, 112.5 (d, J=22.4 Hz), 111.8, 62.5, 62.4, 54.5, 54.0, 49.6, 31.4, 30.1, 22.5, 21.5 ppm.

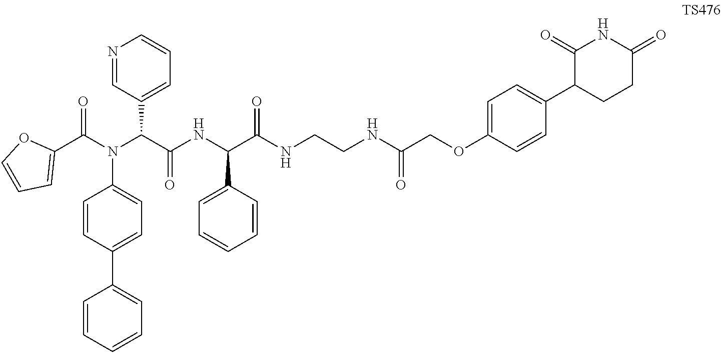 [SMILES: O=C(COc1ccc(C2CCC(=O)NC2=O)cc1)NCCNC(=O)[C@H](NC(=O)[C@@H](c1cccnc1)N(C(=O)c1ccco1)c1ccc(-c2ccccc2)cc1)c1ccccc1]

¹H NMR (700 MHz, DMSO-d₆): δ=10.79 (s, 1H), 8.95 (d, J=7.3 Hz, 1H), 8.48 (s, 1H), 8.39 (s, 1H), 8.36 (t, J=5.4 Hz, 1H), 8.05 (t, J=5.5 Hz, 1H), 7.67 (s, 1H), 7.65-7.59 (m, 3H), 7.54 (d, J=8.0 Hz, 2H), 7.48-7.40 (m, 4H), 7.39-7.32 (m, 3H), 7.30 (t, J=7.3 Hz, 1H), 7.27-7.19 (m, 2H), 7.11 (d, J=8.7 Hz, 2H), 6.88 (d, J=8.7 Hz, 2H), 6.54 (s, 1H), 6.33 (dd, J=3.6, 1.6 Hz, 1H), 5.53 (d, J=3.6 Hz, 1H), 5.47 (d, J=7.2 Hz, 1H), 4.40 (d, J=14.8 Hz, 1H), 4.39 (d, J=14.8 Hz, 1H), 3.77 (dd, J=11.6, 4.9 Hz, 1H), 3.19-3.11 (m, 4H), 2.64 (ddd, J=17.1, 11.8, 5.3 Hz, 1H), 2.47 (dt, J=17.3, 4.4 Hz, 1H), 2.13 (ddd, J=12.1, 12.1, 4.4 Hz, 1H), 1.98 (ddd, J=13.5, 4.9, 4.8 Hz, 1H) ppm.

¹³C NMR (176 MHz, DMSO-d₆): δ=174.9, 173.9, 170.3, 169.0, 168.4, 163.5, 158.7, 157.0, 146.7, 145.9, 140.0, 139.2, 139.1, 139.0, 138.5, 132.3, 132.1, 130.1, 129.4, 128.8, 128.4, 128.1, 127.8, 127.0, 127.0, 123.8, 116.8, 115.0, 111.9, 67.4, 62.5, 57.5, 47.0, 38.8, 38.4, 31.9, 26.4 ppm.

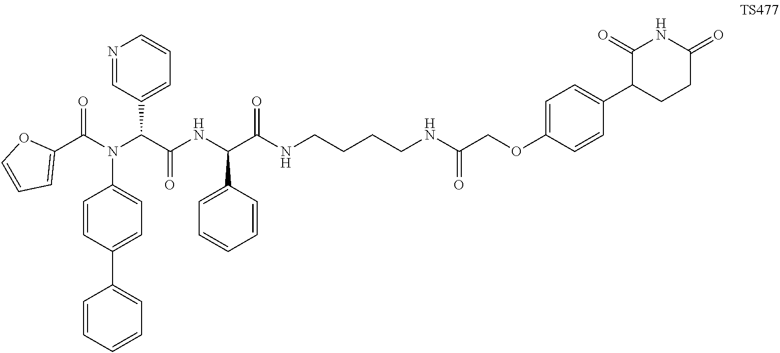 [SMILES: O=C(COc1ccc(C2CCC(=O)NC2=O)cc1)NCCCCNC(=O)[C@H](NC(=O)[C@@H](c1cccnc1)N(C(=O)c1ccco1)c1ccc(-c2ccccc2)cc1)c1ccccc1]

¹H NMR (700 MHz, DMSO-d₆): δ=10.80 (s, 1H), 8.93 (d, J=7.4 Hz, 1H), 8.53 (d, J=1.4 Hz, 0H), 8.44 (s, 1H), 8.24 (t, J=5.7 Hz, 1H), 8.04 (t, J=5.9 Hz, 1H), 7.71 (d, J=7.8 Hz, 1H), 7.68 (dd, J=1.7, 0.8 Hz, 1H), 7.65-7.61 (m, 2H), 7.59-7.50 (m, 2H), 7.46-7.41 (m, 5H), 7.40-7.33 (m, 4H), 7.32-7.17 (m, 2H), 7.12 (d, J=8.4 Hz, 2H), 6.88 (d, J=8.4 Hz, 2H), 6.55 (s, 1H), 6.33 (dd, J=3.6, 1.7 Hz, 1H), 5.55 (d, J=3.6 Hz, 1H), 5.48 (d, J=7.4 Hz, 1H), 4.42 (s, 2H), 3.77 (ddd, J=11.6, 5.0, 2.9 Hz, 1H), 3.11-3.03 (m, 2H), 3.03-2.97 (m, 2H), 2.64 (ddd, J=17.1, 11.9, 5.3 Hz, 1H), 2.49-2.44 (m, 1H), 2.14 (ddd, J=13.0, 11.7, 4.4 Hz, 1H), 1.99 (ddd, J=13.2, 4.9, 4.9 Hz, 1H), 1.37-1.29 (m, 4H) ppm.

¹³C NMR (176 MHz, DMSO-d₆): δ=174.9, 173.9, 169.8, 168.7, 168.0, 158.7, 157.1, 146.7, 145.9, 140.0, 139.1, 139.0, 138.7, 132.3, 132.1, 132.0, 130.0, 129.4, 128.7, 128.6, 128.4, 128.1, 127.7, 127.3, 127.1, 127.0, 116.9, 115.0, 111.9, 67.5, 62.4, 57.4, 47.0, 38.8, 38.3, 31.9, 26.8, 26.7, 26.5 ppm.

### BT150 BT152 and BT153 Experimental:

All reagents and solvents were purchased from commercial sources and used without further purification. Nuclear magnetic resonance spectra were recorded on a Bruker Avance III HD spectrometer operating at 400 MHz for ¹H NMR and 100 MHz for ¹³C NMR. ¹H NMR and ¹³C NMR chemical shifts (S) are reported in parts per million (ppm) and are referenced to residual protium in solvent and to the carbon resonances of the residual solvent peak respectively. DEPT and correlation spectra were run in conjunction to aid assignment. Coupling constants (J) are quoted in Hertz (Hz), and the following abbreviations were used to report multiplicity: s=singlet, d=doublet, dd=doublet of doublets, ddd=double doublet of doublets, t=triplet, q=quartet, m=multiplet, br s=broad singlet. Purification by flash column chromatography was carried out using Fisher Scientific silica gel 60 Å (35-70 μm), or by using Biotage Selekt, Biotage Isolera, Grace Reveleris or Buchi Pure systems. Analytical thin layer chromatography was performed on glass plates pre-coated with silica gel (Analtech, UNIPLATE™ 250 μm/UV254), with visualization being achieved using UV light (254 nm) and/or by staining with alkaline potassium permanganate dip. Reaction monitoring LC-MS analyses were conducted using Agilent InfinityLab LC/MSD systems. Chiral HPLC analysis was conducted using an Agilent 1100 series LC equipped with DAD (G1315B), Column Oven (G1316A), Autosampler (G1313A) with 100 μL loop, Quaternary Pump (G1311A) and an Agilent 1200 series Degasser (G1322A). Data collection and processing was conducted using Agilent Technologies Chemstation B.04.03. SFC purity analyses were conducted using a Waters ACQUITY UPC² system with Binary Solvent Manager (K17C2B854M), Sample Manager (L17C2S782M), Convergence Manager (K17C2M838M), Column Manager (E18AZ3268M) and PDA Detector (M17C2P349A). Data were collected and processed using Empower 3 Build 3471 software. Optical rotations were recorded on a Bellingham & Stanley ADP450 polarimeter.

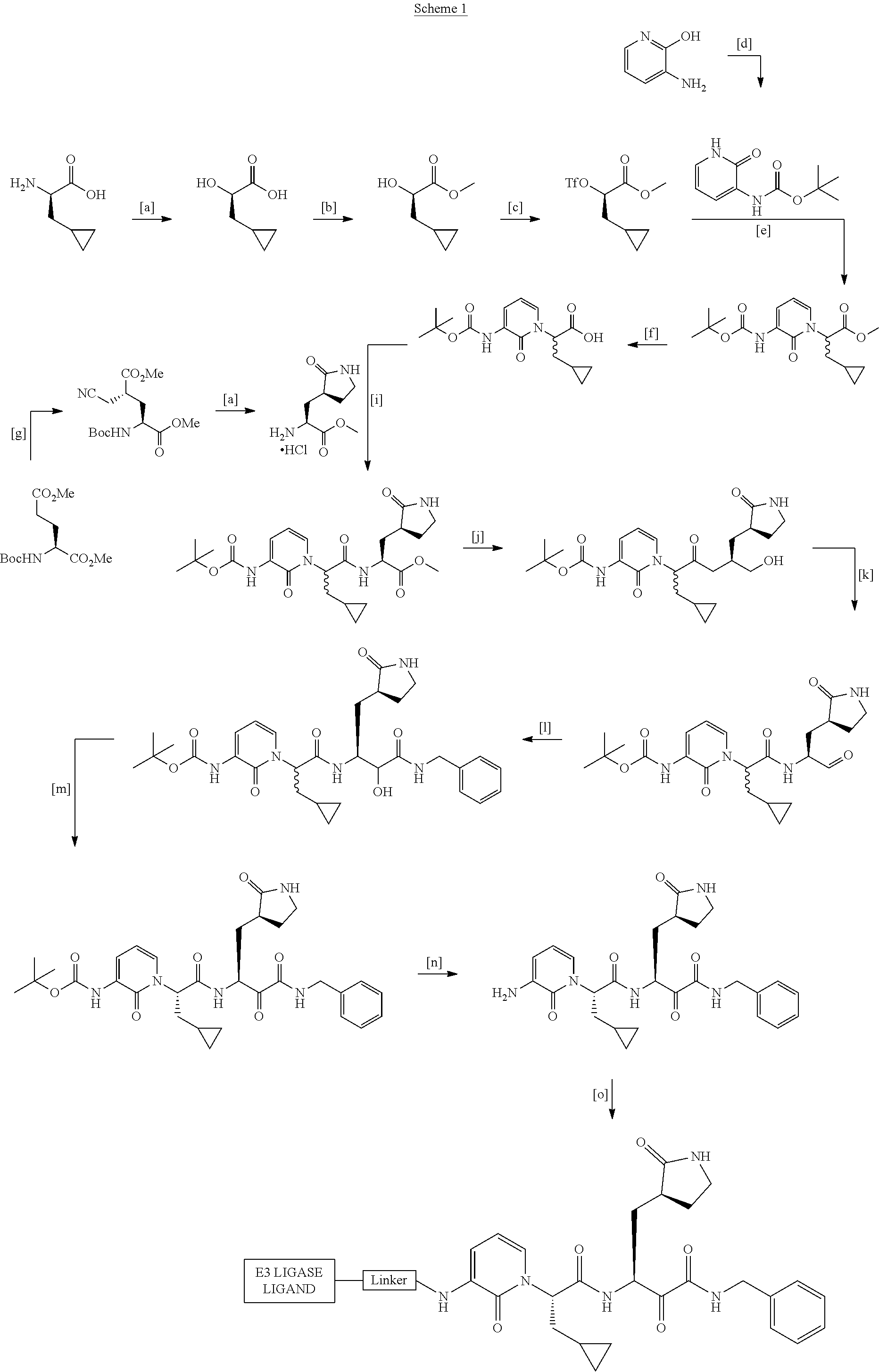

Reagents & Conditions: Conditions: [a] H₂SO₄, NaNO₂, H₂O, 0-5° C.; [b] SOCl₂, MeOH, 0° C.; [c] Tf₂O, 2,6-lutidine, DCM, 0° C.; [d] Boc₂O, THF, reflux; [e] NaH, THF, 0° C.; [f] LiOH·H₂O, MeOH, H₂O, RT; [g] BrCH₂CN, LHMDS, THF, −78° C.; [h] (i) NaBH₄, CoCl₂, MeOH, 0° C.; GO 4M HCl (1,4-dioxane); [i] EDC·HCl, HOBt, TEA, DCM, 0° C.; [j] NaBH₄, MeOH, RT; [k] DMP, NaHCO₃, DCM, RT; [1] (i) benzyl isocyanide, AcOH, DCM, RT; (ii) LiOH·H₂O, MeOH, H₂O, RT; [m] (i) DMP, NaHCO₃, DCM, RT; (ii) prep-HPLC; [n] TFA, DCM; [o] HO₂C-linker-E3Ligand, COMU, DIPEA, DMF.

### (R)-3-Cyclopropyl-2-hydroxypropanoic acid

To a stirred solution of D-cyclopropylalanine (45.00 g, 0.35 mol) in 2M H₂SO₄ (675 mL) at 0° C. was added a solution of sodium nitrite (120.00 g, 1.71 mol) in water (270 mL) in a dropwise fashion over 50 minutes, maintaining the temperature below 5° C. over the course of the addition. The resulting reaction mixture was stirred for 3 hours at 0-5° C., and then allowed to warm to ambient temperature and stirred for 16 hours. The reaction mixture was extracted with TBME (600 mL), and the aqueous phase was further extracted with TBME (5×200 mL). The combined organic extracts were dried over anhydrous magnesium sulfate and concentrated under reduced pressure to afford the title compound as a pale-yellow oil (21.20 g, 47%). ¹H NMR (CDCl₃) δ: 4.36 (dd, J₁=7.0 Hz, J₂=4.8 Hz, 1H), 1.76-1.66 (m, 2H), 0.95-0.82 (m, 1H), 0.60-0.44 (m, 2H), 0.20-0.06 (m, 2H). ¹³C NMR (CDCl₃) δ: 179.90, 70.86, 39.08, 6.70, 4.56, 3.94.

### Methyl (R)-3-cyclopropyl-2-hydroxypropanoate

To a stirred solution of (R)-3-cyclopropyl-2-hydroxypropanoic acid (21.10 g, 162.13 mmol) in methanol at ° C. was added thionyl chloride (23.65 mL, 324.27 mol) in a dropwise fashion over 20 minutes. The resulting solution was allowed to warm to ambient temperature over 2 hours, and was subsequently concentrated under reduced pressure. Purification by flash column chromatography, eluting with 25% EtOAc/petroleum ether (40:60), afforded the title compound as a colourless oil (12.54 g, 54%). ¹H NMR (CDCl₃) δ: 4.28 (dd, J₁=6.4 Hz, J₂=4.6 Hz, 1H), 3.79 (s, 3H), 1.74-1.58 (m, 3H), 0.92-0.80 (m, 1H), 0.53-0.43 (m, 2H), 0.15-0.04 (m, 2H). ¹³C NMR (CDCl₃) δ: 175.69, 70-93, 52.40, 39-39, 6.62, 4.43, 3.88.

### Methyl (R)-3-cyclopropyl-2-(((trifluoromethyl)sulfonyl)oxy)propanoate

To a stirred solution of methyl (R)-3-cyclopropyl-2-hydroxypropanoate (12.50 g, 86.70 mmol) in DCM (163 mL) at ° C. was added 2,6-lutidine (25.10 mL, 215.89 mmol) followed by triflic anhydride (32.50 mL, 193.35 mmol) in a dropwise fashion maintaining the temperature at 0° C. over the course of the addition, and thereafter for a further 40 minutes. The reaction mixture was treated with 1M HCl (aq) (200 mL) and brine (600 mL), and the organic phase was separated. The aqueous component was extracted with DCM (2×100 mL), and the combined organic extracts were dried over anhydrous magnesium sulfate and concentrated under reduced pressure to give a brown oil. The oil was taken up in diethyl ether (300 mL) and further washed with 1M HCl (aq) and brine before being dried over anhydrous magnesium sulfate and concentrated under reduced pressure. This afforded the title compound as a pale brown oil (21.74 g, 91%) which was used directly in the next step with no further purification. ¹H NMR (CDCl₃) δ: 5.20 (dd, J₁=7.8 Hz, J₂=4.5 Hz, 1H), 3.85 (s, 3H), 1.99-1.85 (m, 2H), 0.92-0.81 (m, 1H), 0.65-0.51 (m, 2H), 0.25-0.11 (m, 2H). ¹³C NMR (CDCl₃) δ: 167.73, 118.63 (d, J=320 Hz), 83.97, 53.29, 37.09, 6.67, 5.08, 3.90.

### tert-Buty 1 (2-oxo-1,2-dihydropyridin-3-yl)carbamate

To a stirred solution of 3-amino-2-hydroxypyridine (25.00 g, 227.04 mmol) in THF (530 mL) at ambient temperature was portion-wise added Boc anhydride (49.55 g, 227.04 mmol). The reaction mixture was heated to reflux, and after 4 hours, a further portion of Boc anhydride (39.60 g, 181.63 mmol) was added, with reflux being continued for a further 18 hours. The reaction mixture was cooled to ambient temperature and concentrated under reduced pressure. Purification by flash column chromatography, eluting with 2% MeOH/DCM, afforded the title compound as an off-white solid (25.65 g, 54%). ¹H NMR (CDCl₃) δ: 12.75 (br s, 1H), 8.14 (d, J=6.7 Hz, 1H), 7.57 (s, 1H), 7.03 (dd, J₁=6.6 Hz, J₂=1.7 Hz, 1H), 6.36 (t, J=7.0 Hz, 1H), 1.54 (s, 9H). ¹³C NMR (CDCl₃) δ: 158.96, 152.84, 129.84, 125.49, 122.08, 108.00, 80.96, 28.39.

### Methyl 2-(3-((tert-butoxycarbonyl)amino)-2-oxopyridin-1(2H)-yl)-3-cyclopropylpropanoate

To a stirred solution of tert-butyl (2-oxo-1,2-dihydropyridin-3-yl)carbamate (15.90 g, 75.63 mmol) in THF (475 mL) at 0° C. was portion-wise added sodium hydride (4.69 g, 117.23 mmol, 60% dispersion in mineral oil), and after completion of the addition, the reaction mixture was stirred at ° C. for a further 30 minutes. A solution of methyl (R)-3-cyclopropyl-2-(((trifluoromethyl)sulfonyl)oxy)propanoate (21.58 g, 78.13 mmol) in THF (225 mL) was then added dropwise at such a rate as to maintain the temperature at 0° C., with further stirring thereafter being maintained at this temperature for a total of 43 hours. The reaction mixture was concentrated under reduced pressure. Purification by flash column chromatography, eluting with 20% EtOAc/petroleum ether (40:60), afforded the title compound as a thick yellow oil (12-31 g, 48%). ¹H NMR (DMSO-d₆) δ: 7.85 (dd, J₁=7.5 Hz, J₂=1.1 Hz, 1H), 7.80 (s, 1H), 7.40 (dd, J₁=7.0 Hz, J₂=1.7 Hz, 1H), 6.34 (t, J=7.2 Hz, 1H), 5.23 (dd, J₁=9.8 Hz, J₂=5.2 Hz, 1H), 3.64 (s, 3H), 2.08-1.92 (m, 2H), 1.47 (s, 9H), 0.57-0.43 (m, 1H), 0.39-0.23, m, 2H), 0.11-0.04 (m, 1H), −0.08-−1.15 (m, 1H). ¹³C NMR (DMSO-d₆) δ: 169.54, 156.37, 152.14, 130.44, 128.48, 120.30, 105.48, 80.08, 61.80, 52.21, 33.07, 27.85, 7.36, 4.30, 3.35.

### 2-(3-((tert-Butoxycarbonyl)amino)-2-oxopyridin-1(2H)-yl)-3-cyclopropylpropanoic acid

To a stirred solution of methyl 2-(3-((tert-butoxycarbonyl)amino)-2-oxopyridin-1(2H)-yl)-3-cyclopropylpropanoate (12.30 g, 36.57 mmol) in methanol (346 mL) at ambient temperature was added water (69.2 mL) followed by lithium hydroxide monohydrate (3.07 g, 73.13 mmol), and the resulting reaction mixture was stirred for an hour. The reaction mixture was acidified to pH 6 with 1M HCl (aq), and then partially concentrated under reduced pressure to remove the bulk of the methanol. The resulting aqueous solution was initially extracted with diethyl ether (5×200 mL) which, after drying and concentration of the extracts afforded a small initial crop of product. The aqueous phase was subsequently extracted with DCM and further with 10% MeOH/DCM, these extracts also being dried over anhydrous magnesium sulfate and concentrated under reduced pressure to give further crops. All crops of product were combined and purified by flash column chromatography, eluting with 5% MeOH/DCM increased to 10% MeOH/DCM. This afforded the title compound as a pale yellow solid (8.80 g, 73%). ¹H NMR (DMSO-d₆) δ: 13.08 (br s, 1H), 7.82 (dd, J₁=7.4 Hz, J₂=1.4 Hz, 1H), 7.77 (s, 1H), 7.37 (dd, J₁=7.0 Hz, J₂=1.7 Hz, 1H), 6.31 (t, J=7.2 Hz, 1H), 5.24 (dd, J₁=10.6 Hz, J₂=4.9 Hz, 1H), 2-08-1.89 (m, 2H), 1.47 (s, 9H), 0.55-0.43 (m, 1H), 0.37-0.24 (m, 2H), 0.10-0.03 (m, 1H), −0.05-−0.13 (m, 1H). ¹³C NMR (DMSO-d₆) δ: 171.34, 156.80, 152.13, 129.78, 127.93, 119.36, 104.50, 79.95, 60.77, 35.56, 27.86, 7.97, 4.27, 3.56. m/z (ES+): 343.3 [M+Na⁺]⁺. [α]_{D}²⁴=−0.6° (c=1, MeOH); 1:1 mixture of enantiomers determined by Chiral HPLC (Chiralpak AD-H, 250×4.6 mm, 5 μm, 90% hexane+0.1% TFA isocratic).

### Dimethyl (2S,4R)-2-((tert-butoxycarbonyl)amino)-4-(cyanomethyl)pentanedioate

To a stirred solution of N-Boc L-glutamic acid dimethyl ester (40.00 g, 145.30 mmol) in THF (440 mL) at −70° C. was dropwise added LHMDS (313.84 mL, 313.84 mmol, 1M solution in THF) over 15 minutes maintaining the temperature below −63° C. during the course of the addition. After stirring the resulting reaction mixture at −70° C. for 1 hour, bromoacetonitrile (10.83 mL, 155.47 mmol) was added dropwise via syringe pump over 1 hour maintaining the temperate at or below −70° C. during the course of the addition. After stirring at −70° C. for a further 3.75 hours, cold methanol (29 mL) was added in one portion. After 30 minutes further stirring, a cold mixture of acetic acid (29 mL) in THF (175 mL) was added in one portion, and the reaction mixture was allowed to warm to ambient temperature with stirring overnight. The reaction mixture was poured into brine (720 mL) and the organic phase was separated. The aqueous component was extracted with ethyl acetate (2×500 mL), and the combined organic extracts were dried over anhydrous magnesium sulfate and concentrated under reduced pressure. Purification by flash column chromatography, eluting with 25% EtOAc/petroleum ether (40:60), afforded the title compound as a pale yellow oil (39.36 g, 86%). ¹H NMR (CDCl₃) δ: 5.13 (d, J=7.9 Hz, 1H), 4.44-4.33 (m, 1H), 3.76 (s, 3H), 3.75 (s, 3H), 2.91-2.81 (m, 1H), 2.78 (m, 2H), 2.23-2.10 (m, 2H), 1.44 (s, 9H). ¹³C NMR (CDCl₃) δ: 172.44, 172.18, 155.72, 117.27, 80.74, 52.90, 52.83, 51.17, 38.36, 34.07, 28.37, 19.15.

### Methyl (S)-2-((tert-butoxycarbonyl)amino)-3-((S)-2-oxopyrrolidin-3-yl)propanoate

A stirred solution of dimethyl (2S,4R)-2-((tert-butoxycarbonyl)amino)-4-(cyanomethyl)pentanedioate (39.00 g, 124.07 mmol) in methanol (730 mL) was treated with cobalt chloride hexahydrate (14.76 g, 62.04 mmol) and then cooled to 0° C. Sodium borohydride (18.78 g, 496.29 mmol) was added portion-wise over 1 hour maintaining the temperature below +2° C. during the course of the addition. The reaction mixture was allowed to slowly warm to ambient temperature and was stirred for 22 hours before being concentrated under reduced pressure. The resulting brown oil was partitioned between ethyl acetate (1500 mL), brine (720 mL) and 2M HCl (aq) (360 mL). The organic phase was separated, and the aqueous component was extracted with ethyl acetate (2×400 mL). The combined organic extracts were washed with brine (200 mL), dried over anhydrous magnesium sulfate and concentrated under reduced pressure. Purification by flash column chromatography, eluting with 4% MeOH/DCM, and subsequent trituration with petroleum ether (40:60) afforded the title compound as an off-white solid (14.98 g, 52.32 mmol, 42%). ¹H NMR (CDCl₃) δ: 5.76 (br s, 1H), 5.48 (d, J=7.9 Hz, 1H), 4.38-4.28 (m, 1H), 3.75 (s, 3H), 3.41-3.31 (m, 2H), 3.54-3.42 (m, 2H), 2.20-2.09 (m, 1H), 1.94-1.80 (m, 2H), 1.45 (s, 9H). ¹³C NMR (CDCl₃) δ: 179.76, 173.02, 155.88, 80.05, 52.55, 52.38, 40.51, 38.23, 34.29, 28.42, 28.25. m/z (ES+): 309.2 [M+Na⁺]⁺

### Methyl (S)-2-amino-3-((S)-2-oxopyrrolidin-3-yl)propanoate hydrochloride

Methyl (S)-2-((tert-butoxycarbonyl)amino)-3-((S)-2-oxopyrrolidin-3-yl)propanoate (12.96 g, 45.26 mmol) was treated with hydrogen chloride (226 mL, 905.27 mmol, 4M solution in 1,4-dioxane) and the resulting mixture was stirred at ambient temperature for 2 hours before being concentrated under reduced pressure. The residue was triturated with diethyl ether to afford the title compound as a colourless solid (10.08 g, quant.). ¹H NMR (DMSO-d₆) δ: 8.81 (br s, 3H), 7.96 (s, 1H), 4.17 (m, 1H), 3.76 (s, 3H), 3.26-3.14 (m, 2H), 2.66-2.56 (m, 1H), 2.35-2.25 (m, 1H), 2.14-2.04 (m, 1H), 1.92-1.83 (m, 1H), 1.73-1.61 (m, 1H). ¹³C NMR (DMSO-d6) δ: 178.03, 169.76, 66.37, 52.83, 50.99, 38.10, 31.77, 27.68. [α]_{D}²⁴=+45.6° (c=1.0, MeOH)

### Methyl (S)-2-(2-(3-((tert-butoxycarbonyl)amino)-2-oxopyridin-1(2H)-yl)-3-cyclopropylpropanamido)-3-((S)-2-oxopyrrolidin-3-yl)propanoate

To a stirred solution of 2-(3-((tert-butoxycarbonyl)amino)-2-oxopyridin-1(2H)-yl)-3-cyclopropylpropanoic acid (8.55 g, 26.52 mmol) in DCM (200 mL) at 0° C. was added HOBt (4.93 g, 29.18 mmol) and EDC·HCl (5.59 g, 29.18 mmol). After stirring at 0° C. for 1 hour a solution containing a mixture of methyl (S)-2-amino-3-((S)-2-oxopyrrolidin-3-yl)propanoate hydrochloride (6.19 g, 27.85 mmol) and triethylamine (2.95 g, 29.18 mmol) in dichloromethane (120 mL) was added dropwise. Further triethylamine was added dropwise to adjust the reaction mixture to pH 9, after which point stirring was maintained at ° C. overnight. After 18 hours the reaction mixture was warmed to ambient temperature over 6 hours. TLC indicated the reaction mixture contained a mixture of the acid starting material and the product. Additional portions of EDC·HCl (1.53 g, 7.98 mmol) and HOBt (1.34 g, 7.98 mmol) were added, followed by (S)-2-amino-3-((S)-2-oxopyrrolidin-3-yl)propanoate hydrochloride (2.43 g, 7.98 mmol) and triethylamine (0.85 g, 8.40 mmol). After 42 hours the reaction mixture was treated with water (200 mL) and the organic phase was separated. The aqueous component was extracted with DCM (100 mL) and the combined organic extracts were washed with saturated sodium bicarbonate solution (2×200 mL), dried over anhydrous magnesium sulfate and concentrated under reduced pressure. Purification by flash column chromatography, eluting with 3% MeOH/DCM, and subsequent trituration with petroleum ether (40:60) afforded the title compound as a white solid (8.87 g, 68%). ¹H NMR (DMSO-d₆) δ: 8.88 (dd, J₁=19.2 Hz, J₂=7.3 Hz, 1H), 7.79 (d, J=7.4 Hz, 1H), 6.29 (t, J=7.2 Hz, 1H), 7.75 (d, J=2.4 Hz, 1H), 7.66 (d, J=10.1 Hz, 1H), 6.29 (t, J=7.1 Hz, 1H), 5.72-5.56 (m, 1H), 4.34-4.24 (m, 1H), 3.61 (d, J=10.0 Hz, 3H), 3.19-3.03 (m, 2H), 2.30-2.17 (m, 1H), 2.16-1.87 (m, 3H), 1.85-1.71 (m, 1H), 1.67-1.53 (m, 2H), 1.46 (s, 9H), 0.57-0.41 (m, 1H), 0.36-0.27 (m, 2H), 0.17-0.09 (m, 1H), 0.03-−0.05 (m, 1H). ¹³C NMR (DMSO-d₆) δ: 177.85, 177.84, 172.08, 172.06, 169.39, 169.21, 156.83, 156.78, 152.18, 128.84, 128.80, 127.98, 127.97, 119.94, 119.87, 80.09, 58.06, 57.57, 51.98, 51.97, 50.58, 50.53, 37.64, 37.58, 35.66, 35.19, 32.37, 32.11, 27.88, 27.19, 27.10, 22.48, 19.87, 19.27, 18.64, 14.16, 11.24, 7.52, 7.49, 4.58, 4.43, 3.58, 3.51. m/z (ES+): 513.3 [M+Na⁺]⁺

### tert-Buty 1 (1-(-3-cyclopropyl-1-(((S)-1-hydroxy-3-((S)-2-oxopyrrolidin-3-yl)propan-2-yl)amino)-1-oxopropan-2-yl)-2-oxo-1,2-dihydropyridin-3-yl)carbamate

To a stirred solution of methyl (S)-2-(2-(3-((tert-butoxycarbonyl)amino)-2-oxopyridin-1(2H)-yl)-3-cyclopropylpropanamido)-3-((S)-2-oxopyrrolidin-3-yl)propanoate (8.70 g, 17-73 mmol) in methanol (180 mL) at ambient temperature was portion-wise added sodium borohydride (3.35 g, 88.68 mmol) over 20 minutes at such a rate so as to maintain the temperature around 20° C. The reaction mixture was stirred for 1.5 hours and then treated firstly slowly with water (20 mL) and then adjusted to pH 9 by dropwise addition of 1M hydrochloric acid. The reaction mixture was partially concentrated to a volume of about 50 mL, and then extracted with dichloromethane (3×30 mL). The combined organic extracts were dried over anhydrous magnesium sulfate and concentrated under reduced pressure to give the title compound as a white solid (8.13 g, 99%). ¹H NMR (DMSO-d₆) δ: 8.10 (dd, J₁=20.8 Hz, J₂=8.6 Hz, 1H), 7.78 (d, J=6.8 Hz, 1H), 7.73 (s, 1H), 7.53 (d, J=15.7 Hz, 1H), 7.44-7.37 (m, 1H), 6.28 (td, J₁=7.2 Hz, J₂=2.2 Hz, 1H), 5.62-5.49 (m, 1H), 4.67 (m, 1H), 3.76 (m, 1H), 3.39-3.20 (m, 2H), 3.19-1.98 (m, 2H), 2.21-1.86 (m, 3H), 1.85-1.69 (m, 2H), 1.64-1.28 (m, 2H), 1.46 (s, 9H), 0.55-0.40 (m, 1H), 0.37-0.24 (m, 2H), 0.19-0.07 (m, 1H), 0.03-−0.05 (m, 1H). ¹³C NMR (DMSO-d₆) δ: 178.73, 178.69, 168.87, 168.77, 156.79, 152.18, 129.02, 128.71, 127.96, 127.93, 119.86, 104.81, 104.74, 80.09, 63.67, 63.62, 58.33, 58.10, 49.17, 37.72, 37.51, 35.78, 35.48, 32-34, 32.23, 27.90, 27.62, 27-54, 7.57, 4-51, 4.46, 3.67, 3.53. m/z (ES+): 485.3 [M+Na⁺]⁺

### tert-Buty 1 (1-(3-cyclopropyl-1-oxo-1-(((S)-1-oxo-3-((S)-2-oxopyrrolidin-3-yl)propan-2-yl)amino)propan-2-yl)-2-oxo-1,2-dihydropyridin-3-yl)carbamate

To a stirred solution of tert-butyl (1-(3-cyclopropyl-1-(((S)-1-hydroxy-3-((S)-2-oxopyrrolidin-3-yl)propan-2-yl)amino)-1-oxopropan-2-yl)-2-oxo-1,2-dihydropyridin-3-yl)carbamate (8.00 g, 17.30 mmol) in DCM (800 mL) at ambient temperature was added NaHCO₃ (0.509 g, 6.05 mmol) and Dess-Martin periodinane (9.17 g, 216.20 mmol) and the solution was stirred for 1 hour. The reaction mixture was treated with a saturated aqueous solution of NaHCO₃ (200 mL) and the reaction mixture was filtered through Celite™. The organic phase was separated, and the aqueous component was extracted with DCM (2×50 mL). The combined organic extracts were dried over anhydrous magnesium sulfate and concentrated under reduced pressure to a yellow solid. Purification by flash column chromatography, eluting with 4% MeOH/DCM, afforded the title compound as a white solid (7.17 g, 90%). ¹H NMR (DMSO-d6) δ: 9.40 (d, J=10.1 Hz, 1H), 8.81 (dd, J₁=12.9 Hz, J₂=7.3 Hz, 1H), 7.86-7.67 (m, 2H), 7.67-7.50 (m, 1H), 7.43-7.37 (m, 1H), 6.33-6.25 (m, 1H), 5.68-5.54 (m, 1H), 4.37-4.13 (m, 1H), 3.24-2.96 (m, 3H), 2.31-1.28 (m, 6H), 1.46 (s, 9H), 0.57-0.39 (m, 1H), 0.37-0.23 (m, 2H), 0.20-0.07 (m, 1H), 0.04-−0.08 (m, 1H). m/z (ES−): 459.3 [M−H⁺]⁻

### (3S)-1-(Benzylamino)-3-(2-(3-((tert-butoxycarbonyl)amino)-2-oxopyridin-1(2H)-yl)-3-cyclopropylpropanaimido)-1-oxo-4-((S)-2-oxopyrrolidin-3-yl)butan-2-y 1 acetate

To a stirred solution of tert-butyl (1-(3-cyclopropyl-1-oxo-1-(((S)-1-oxo-3-((S)-2-oxopyrrolidin-3-yl)propan-2-yl)amino)propan-2-yl)-2-oxo-1,2-dihydropyridin-3-yl)carbamate (7.10 g, 15.42 mmol) and acetic acid (1.85 g, 30.83 mmol) in DCM (710 mL) at ambient temperature was added benzyl isocyanide (1.81 g, 15.42 mmol) and the reaction mixture was stirred for 22 hours before being concentrated under reduced pressure. Purification by flash column chromatography, eluting with 3% MeOH/DCM, afforded the title compound as an off-white solid (8-37 g, 85%). ¹H NMR (DMSO-d₆) δ: 8.68-8.23 (m, 2H), 7.81 (m, 1H), 7.73 (d, J=17.6 Hz, 1H), 7.60 (m, 1H), 7.47-7.16 (m, 6H), 6.35-6.26 (m, 1H), 5.65-5.52 (m, 1H), 5.16-4.85 (m, 1H), 4.35-4.14 (m, 3H), 3.16-2.98 (m, 2H), 2.22-1.58 (m, 8H), 1.47 (s, 9H), 1.57-1.02 (m, 2H), 0.55-0.42 (m, 1H), 0.37-0.27 (m, 2H), 0.19-0.09 (m, 1H), 0.05-−0.07 (m, 1H). m/z (ES+): 660.4 [M+Na⁺]⁺

### tert-Buty 1 (1-(1-(((2S)-4-(benzylamino)-3-hydroxy-4-oxo-1-((S)-2-oxopyrrolidin-3-yl)butan-2-yl)amino)-3-cyclopropyl-1-oxopropan-2-yl)-2-oxo-1,2-dihydropyridin-3-yl)carbamate

A stirred solution of (3S)-1-(benzylamino)-3-(2-(3-((tert-butoxycarbonyl)amino)-2-oxopyridin-1(2H)-yl)-3-cyclopropylpropanamido)-1-oxo-4-((S)-2-oxopyrrolidin-3-yl)butan-2-yl acetate (28.25 g, 44.30 mmol) in methanol (2000 mL) and water (400 mL) was treated with lithium hydroxide monohydrate (3.72 g, 88.60 mmol) and the resulting solution was stirred at ambient temperature for 1.5 hours. The reaction mixture was acidified to pH 6-7 with 1M hydrochloric acid and then partially concentrated under reduced pressure to remove the bulk of the methanol. The resulting solution was extracted with DCM (3×500 mL). The combined organic extracts were washed with brine (250 mL) dried over anhydrous magnesium sulfate and concentrated under reduced pressure to afford the title compound (a mixture of diastereomers) as an off-white foam (24.25 g, 92%). ¹H NMR (DMSO-d₆) δ: 8.40-7.70 (m, 4H), 7.58-7.15 (m, 7H), 6.31-6.26 (m, 1H), 5.86-5.78 (m, 1H), 5.75 (s, 1H), 5.70-5.54 (m, 1H), 4.32-3.88 (m, 4H), 3.19-2.96 (m, 2H), 2.26-0.91 (m, 6H), 1.45 (s, 9H), 0.52-0.36 (m, 1H), 0.34-0.23 (m, 2H), 0.18-0.9 (m, 1H), 0.01-−0.11 (m, 1H). m/z (ES−): 594.3 [M−H⁺]⁻

### tert-Buty 1 (1-(-1-(((S)-4-(benzylamino)-3,4-dioxo-1-((S)-2-oxopyrrolidin-3-yl)butan-2-yl)amino)-3-cyclopropyl-1-oxopropan-2-yl)-2-oxo-1,2-dihydropyridin-3-yl)carbamate

To a stirred solution of tert-butyl (1-(1-(((2S)-4-(benzylamino)-3-hydroxy-4-oxo-1-((S)-2-oxopyrrolidin-3-yl)butan-2-yl)amino)-3-cyclopropyl-1-oxopropan-2-yl)-2-oxo-1,2-dihydropyridin-3-yl)carbamate (3.60 g, 6.04 mmol) in DCM (436 mL) was added sodium hydrogen carbonate (0.22 g, 2.60 mmol) and Dess-Martin periodinane (3.33 g, 7.86 mmol), and the resulting mixture was stirred at ambient temperature for 1 hour. Following the addition of saturated aqueous NaHCO₃ solution (150 mL), the organic phase was separated, and the aqueous component was extracted with DCM (2×150 mL). The combined organic extracts were washed with brine, dried over anhydrous magnesium sulfate, and concentrated under reduced pressure. Purification by flash column chromatography, eluting with 2% MeOH/DCM, afforded the title compound as an off-white solid (3.59 g, quant.).

### tert-Buty 1 (1-((S)-1-(((S)-4-(benzylamino)-3,4-dioxo-1-((S)-2-oxopyrrolidin-3-yl)butan-2-yl)amino)-3-cyclopropyl-1-oxopropan-2-yl)-2-oxo-1,2-dihydropyridin-3-yl)carbamate, (S,S,S)-M_{PL}13b-K

Recrystallisation from acetonitrile afforded an off-white solid which was removed by filtration. Concentration of the filtrate and purification by preparative HPLC afforded the title compound as a pale pink solid. ¹H NMR (DMSO-d₆) (120° C.) δ: 8.63 (m, 1H), 8.51 (d, J=6.6 Hz, 1H), 7.77 (dd, J₁=7.7 Hz, J₂=1.7 Hz, 1H), 7.57 (s, 1H), 7.34-7.21 (m, 6H), 7.16 (s, 1H), 6.25 (t, J=7.3 Hz, 1H), 5.60 (dd, J₁=8.9 Hz, J₂=6.0 Hz, 1H), 5.05-4.99 (m, 1H), 4.36 (d, J=6.6 Hz, 1H), 3.20-3.09 (m, 2H), 2.36-2.26 (m, 1H), 2.21-2.12 (m, 1H), 2.06-1.92 (m, 2H), 1.86-1.63 (m, 3H), 1.50 (s, 9H), 0.66-0.55 (m, 1H), 0.41-0.33 (m, 1H), 0.15-0.09 (m, 1H), 0.04-−0.02 (m 1H). ¹³C NMR (DMSO-d₆) δ: 196.20, 177.88, 169.40, 160.73, 156.82, 152.17, 138.46, 128.75, 128.28, 128.08, 128.97, 127.40, 127.30, 126.93, 119.93, 104.85, 80.08, 57.79, 52.74, 42.02, 37.78, 35.42, 30.91, 27.89, 27.09, 7.48, 4.61, 3.55. HRMS (ES−) calculated for [C₃₁H₃₉N₅O₇—H⁺]⁻ found: 592.2771. [α]_{D}²⁶=−73.1° (c=0.5, MeOH); >97% purity determined by SFC (Trefoil AMY1 (150×3.0 nm, 2.5 μm), IPA:CO₂ (20-60% IPA isocratic)).

### (S)-3-((S)-2-(3-Amino-2-oxopyridin-1(2H)-yl)-3-cyclopropylpropanamido)-N-benzyl-2-oxo-4-((S)-2-oxopyrrolidin-3-yl)butanamide

To a stirred suspension of tert-Butyl (1-((S)-1-(((S)-4-(benzylamino)-3,4-dioxo-1-((S)-2-oxopyrrolidin-3-yl)butan-2-yl)amino)-3-cyclopropyl-1-oxopropan-2-yl)-2-oxo-1,2-dihydropyridin-3-yl)carbamate (200 mg, 0.30 mmol) in DCM (1.5 mL) at ambient temperature was added TFA (0.5 mL). The resulting solution was stirred at ambient temperature for one hour and then concentrated under reduced pressure. The residue was purified by flash column chromatography, eluting with 5% MeOH/DCM in the presence of NH4OH, which afforded the title compound as a pale green solid (101 mg, 68%).

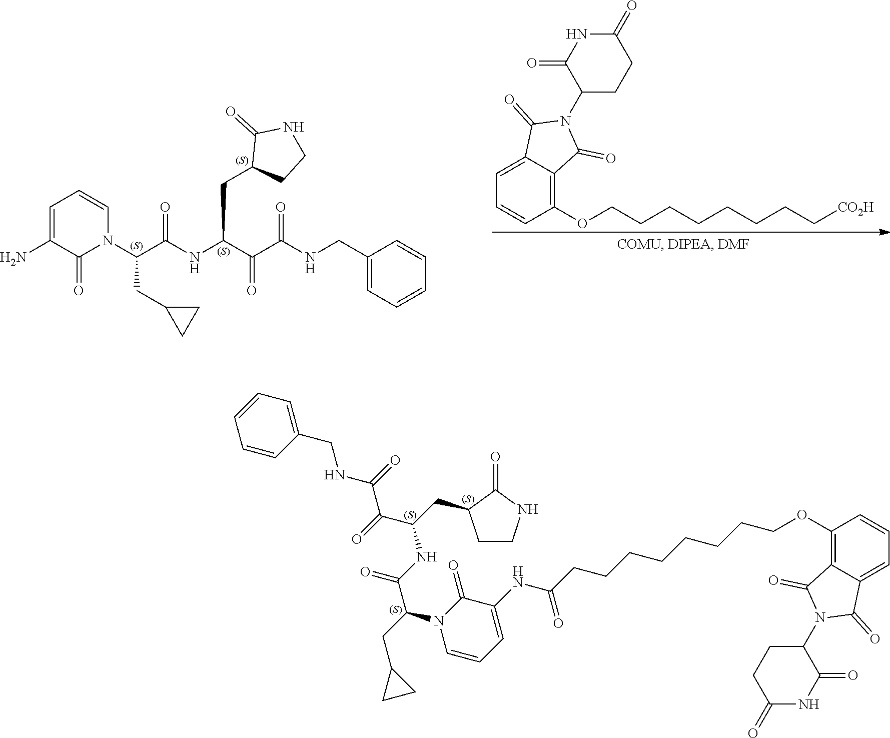

### N-(1-((S)-1-(((S)-4-(benzylamino)-3,4-dioxo-1-((S)-2-oxopyrrolidin-3-yl)butan-2-yl)amino)-3-cyclopropyl-1-oxopropan-2-yl)-2-oxo-1,2-dihydropyridin-3-yl)-9-((2-(2,6-dioxopiperidin-3-yl)-1,3-dioxoisoindolin-4-yl)oxy)nonanamide (BT-153)

To a stirred solution of 9-[2-(2,6-dioxo-3-piperidyl)-1,3-dioxo-isoindolin-4-yl]oxynonanoic acid (78.5 mg, 0.18 mmol) in DMF (3 mL) at ambient temperature was added COMU (102 mg) and DIPEA (78 mg). After 10 minutes, the reaction mixture was treated with a solution of (S)-3-((S)-2-(3-amino-2-oxopyridin-1(2H)-yl)-3-cyclopropylpropanamido)-N-benzyl-2-oxo-4-((S)-2-oxopyrrolidin-3-yl)butanamide (90 mg, 0.18 mmol) in DMF (2 mL), and the reaction mixture was stirred overnight. After dilution of the reaction mixture with H2O/DCM (40 mL, 1:1) and stirring for 10 minutes, the organic phase was separated. The aqueous component was extracted with DCM (2×20 mL), and the combined organic extracts were successively washed with NaHCO₃ (sat aq) (20 mL) and brine (4×20 mL) before being dried over anhydrous magnesium sulfate and concentrated under reduced pressure. Purification by flash column chromatography, eluting with 2-3% MeOH/DCM, afforded the title compound as gummy solid. Lyophilisation from H2O/MeCN (1:1) afforded the title compound as a pale beige solid (43 mg).

¹H NMR (DMSO-d6) (120° C.) δ: 10.56 (s, 1H), 8.70-8.45 (m, 3H), 8.14 (dd, J=7.3, 1.6, 1H), 7.77 (dd, J=8.3, 7.2, 1H), 7.46 (d, J=8.5, 1H), 7.42 (dd, J=7.2, 0.5, 1H), 7.35-7.22 (m, 7H), 6.36-6.19 (m, 1H), 5.66-5.58 (m, 1H), 5.16-4.98 (m, 2H), 4.38-4.31 (m, 2H), 4.25 (t, J=6.5, 2H), 3.22-3.08 (m, 2H), 2.92-2.80 (m, 1H), 2.68-2.51 (m, 2H), 2.41 (t, J=7.4, 2H), 2.37-1.90 (m, 5H), 1.88-1.44 (m, 10H), 1.42-1.26 (m, 3H), 0.67-0.54 (m, 1H), 0.41-0.33 (m, 2H), 0.16-−0.02 (m, 2H)

13C NMR (DMSO-d6) δ: 196.71, 178.39, 173.26, 172.66, 170.44, 169.97, 167.33, 165.78, 161.20, 157.44, 156.50, 138.95, 137.50, 133.72, 129.97, 128.77, 128.57, 127.89, 127.79, 127.43, 127.15, 120.27, 116.69, 115.59, 105.16, 94.86, 69.27, 53.24, 49.20, 42.51, 40.99, 38.29, 36.63, 31.43, 29.22, 29.04, 28.87, 27.57, 25.74, 25.60, 22.47, 8.01, 5.12, 4.03 m/z (ES+): 928.24 [M+Na⁺]⁺

The same conditions were analogously applied to synthesise BT150 and BT152 derivatives.

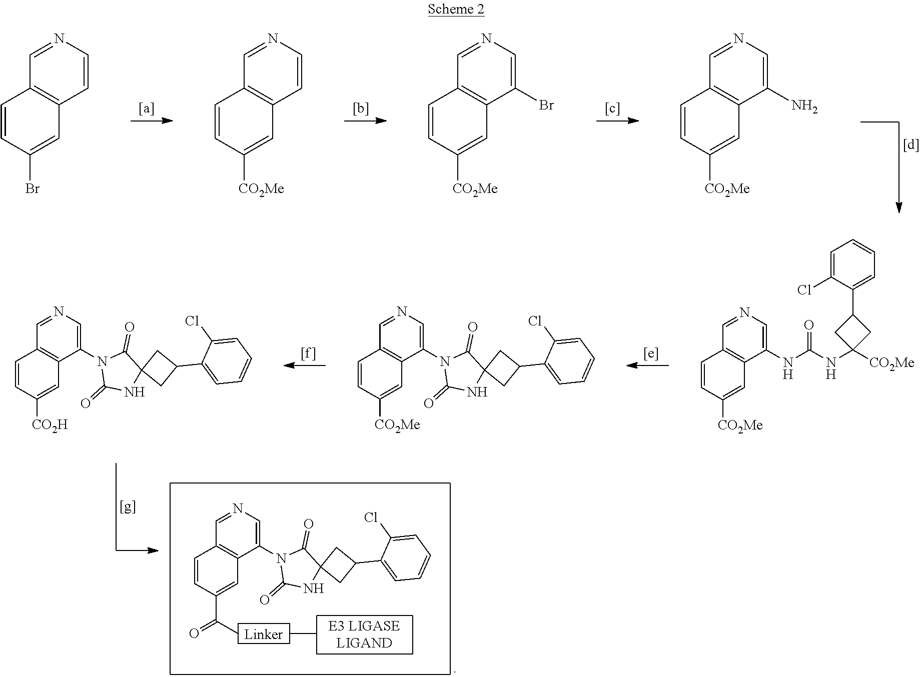

Reagents & Conditions: [a] Pd(OAc)₂, NaOAc, PPh₃, DMF, MeOH, CO, 3.5 bar, 100° C.; [b] NBS, AcOH, 90° C.; [c] NH₂Boc, Pd(OAc)₂, Xantphos, Cs₂CO₃, 1,4-dioxane; [d] (i) triphosgene, Et₃N, DCM; (ii) 1-amino-3-(2-chlorophenyl)cyclobutanecarboxylate hydrochloride; [e] (i) NaOH (s), MeOH; (ii) TFA, DCM; [f] (i) LiOH (aq), THF; (ii) TFA, DCM; [g] H₂N-linker-E3Ligand, HATU, DMAP (cat), DIPEA, DMF, N.B. The same conditions were analogously applied to synthesise 7-substituted isoquinoline carboxylate derivatives.

### Methyl isoquinoline-6-carboxylate

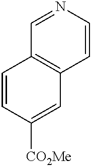 [SMILES: COC(=O)c1ccc2cnccc2c1]

To a stirred solution of 6-bromoisoquinoline (25.00 g, 120.16 mmol), sodium acetate (9.86 g, 120.16 mmol), triphenylphosphine (31.52 g, 120.16 mmol) in DMF (150 mL) and methanol (75 mL) in a steel vessel was added palladium (II) acetate (13.50 g, 60.01 mmol), and the resulting mixture was placed under an atmosphere of CO at 3.5 bar, and heated at 100° C. overnight. Upon cooling to ambient temperature, the reaction mixture was concentrated under reduced pressure, and the residue was partitioned between ethyl acetate (500 mL) and brine (500 mL). The organic phase was separated and the aqueous component was extracted with ethyl acetate (200 mL), before the combined organic extracts were dried over anhydrous magnesium sulfate and concentrated under reduced pressure. Purification by flash column chromatography, eluting with 0-2% MeOH/DCM, afforded the crude title compound as a yellow solid (27.00 g) which was used directly in the next stage without further purification. m/z (ES+): 188.10 [M+H⁺]⁺

Analogously, methyl isoquinoline-7-carboxylate was prepared from 7-bromoisoquinoline according to the same procedure.

### Methyl 4-bromoisoquinoline-6-carboxylate

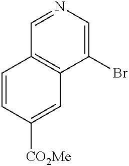 [SMILES: COC(=O)c1ccc2cncc(Br)c2c1]

To a stirred solution of methyl isoquinoline-6-carboxylate (27.00 g) in acetic acid (250 mL) was added NBS (18.74 g, 105.29 mmol) and the resulting reaction mixture was heated at 90° C. overnight. After cooling to ambient temperature, the reaction mixture was concentrated under reduced pressure, and the residue was dissolved in ethyl acetate (100 mL). The solution was washed with saturated aqueous sodium hydrogen carbonate solution (100 mL) and brine (100 mL) before being dried over anhydrous magnesium sulfate and concentrated under reduced pressure. Purification by flash column chromatography, eluting with 40-60% EtOAc/petroleum ether (40-60), afforded the title compound as a yellow solid (12.66 g, 40% over two stages). ¹H NMR (CDCl₃) δ: 9.25 (s, 1H), 8.87 (s, 1H), 8.79 (s, 1H), 8.27 (dd, J=8.5, 1.4, 1H), 8.07 (d, J=8.5, 1H), 4.04 (s, 3H); m/z (ES+): 265.9 [M+H⁺]⁺

Analogously, methyl 4-bromoisoquinoline-7-carboxylate was prepared from methyl isoquinoline-7-carboxylate according to the same procedure. ¹H NMR (CDCl₃) δ: 9.30 (s, 1H), 8.82 (s, 1H), 8.76 (d, J=1.6, 1H), 8.43 (dd, J=8.8, 1.6, 1H), 8.24 (d, J=8.8, 1H), 4.03 (s, 3H); m/z (ES+): 266 [M+H⁺]⁺

### Methyl 4-aminoisoquinoline-6-carboxylate

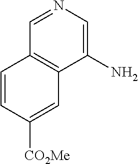 [SMILES: COC(=O)c1ccc2cncc(N)c2c1]

To a stirred solution of methyl 4-bromoisoquinoline-6-carboxylate (12.50 g, 46.98 mmol) in 1,4-dioxane (225 mL) was added palladium (II) acetate (1.01 g, 4.51 mmol), Xantphos (2.61 g, 4.51 mmol), cesium carbonate (22.04 g, 67.65 mmol) and tert-butyl carbamate (26.42 g, 225.49 mmol) in that order, with the resulting mixture being heated in a sealed vessel at 165° C. for 16 hours. Upon cooling to ambient temperature, the reaction mixture was treated with brine (200 mL) and the mixture was extracted with ethyl acetate (3×200 mL). The combined organic extracts were filtered through diatomaceous earth, the filter cake being well-washed with ethyl acetate, and the filtrate was dried over anhydrous magnesium sulfate and concentrated under reduced pressure. Purification by flash column chromatography, eluting with 0-5% MeOH/DCM, afforded the title compound as a yellow solid (2.95 g, 32%). ¹H NMR (DMSO-d6) δ: 8.85 (d, J=0.8, 1H), 8.54 (s, 1H), 8.01 (d, J=0.8, 2H), 7.97 (s, 1H), 6.24 (s, 2H), 3.93 (s, 3H); m/z (ES+): 203.2 [M+H⁺]⁺

Analogously, methyl 4-aminoisoquinoline-7-carboxylate was prepared from methyl 4-bromoisoquinoline-7-carboxylate according to the same procedure. ¹H NMR (DMSO-d6) δ: 8.68 (s, 1H), 8.61 (d, J=1.6, 1H), 8.23 (d, J=8.8, 1H), 8.06 (dd, J=8.8, 1.6, 1H), 7.99 (s, 1H), 6.03 (s, 2H), 3.92 (s, 3H); m/z (ES+): 203.2 [M+H⁺]⁺

### Methyl 4-(3-(3-(2-chlorophenyl)-1-(methoxycarbonyl)cyclobutyl)ureido)isoquinoline-6-carboxylate

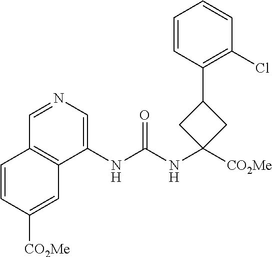 [SMILES: COC(=O)c1ccc2cncc(NC(=O)NC3(C(=O)OC)CC(c4ccccc4Cl)C3)c2c1]

To a stirred solution of methyl 4-aminoisoquinoline-6-carboxylate (2.20 g, 10.88 mmol) and triethylamine (4.54 mL, 32.64 mmol) in DCM (150 mL) at 0° C. was added a solution of triphosgene (1.61 g, 5.44 mmol) in DCM (5 mL) in a dropwise fashion, and the resulting solution was stirred for 45 mins maintaining the temperature at 0° C. After this time, methyl 1-amino-3-(2-chlorophenyl)cyclobutanecarboxylate hydrochloride (3.00 g, 10.88 mmol) was added to the reaction mixture in one portion, and the mixture was allowed to warm to ambient temperature with stirring overnight. The reaction mixture was diluted with DCM (100 mL) and washed with brine before the organics were dried over anhydrous magnesium sulfate, concentrated under reduced pressure and dried under high vacuum. This afforded the crude title compound (urea intermediate) (5.30 g) which was used directly in the next stage without further purification. m/z (ES+): 468.20 [M+H⁺]⁺; 490.20 [M+Na⁺]⁺ Analogously, methyl 4-(3-(3-(2-chlorophenyl)-1-(methoxycarbonyl)cyclobutyl)ureido)isoquinoline-7-carboxylate was prepared from methyl 4-aminoisoquinoline-7-carboxylate according to the same procedure.

### Methyl 4-(2-(2-chlorophenyl)-6,8-dioxo-5,7-diazaspiro[3.4]octan-7-yl)isoquinoline-6-carboxylate

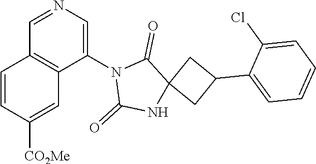 [SMILES: COC(=O)c1ccc2cncc(N3C(=O)NC4(CC(c5ccccc5Cl)C4)C3=O)c2c1]

A stirred solution of methyl 4-(3-(3-(2-chlorophenyl)-1-(methoxycarbonyl)cyclobutyl)ureido)isoquinoline-6-carboxylate (5.30 g) in methanol (100 mL) at 0° C. was treated with sodium hydroxide (0.56 g, 13.93 mmol, pellets finely crushed to granular powder before addition), and the resulting mixture was allowed to warm to ambient temperature with stirring over 45 mins. LCMS analysis indicated complete ester hydrolysis and partial cyclization. The reaction mixture was neutralised by dropwise addition of TFA, and then concentrated under reduced pressure. The residue was treated with DCM (150 mL) and TFA (25 mL) and heated at 60° C. for 2 hours and then allowed to cool to ambient temperature with stirring overnight. The reaction mixture was concentrated under reduced pressure and the residue redissolved in DCM (200 mL). The solution was washed with saturated aqueous sodium hydrogen carbonate solution (100 mL) before being dried over anhydrous magnesium sulfate and concentrated under reduced pressure. Purification by flash column chromatography, eluting with 50% EtOAc/DCM, afforded the title compound as a yellow solid (2.38 g, 50% over two stages). ¹H NMR (DMSO-d6) δ: 9.58 (s, 1H), 9.09 (s, 1H), 8.71 (s, 1H), 8.44 (d, J=8.6, 1H), 8.29 (s, 1H), 8.23 (dd, J=8.6, 1.4, 1H), 7.53 (d, J=7.0, 1H), 7.44 (dd, J=13.8, 7.5, 2H), 7.34-7.28 (m, 1H), 3.94 (s, 3H), 3.92-3.83 (m, 1H), 3.20-3.07 (m, 2H), 2.73-2.59 (m, 2H); m/z (ES+): 436.10 [M+H⁺]⁺

Analogously, methyl 4-(2-(2-chlorophenyl)-6,8-dioxo-5,7-diazaspiro[3,4]octan-7-yl)isoquinoline-7-carboxylate was prepared from methyl 4-(3-(3-(2-chlorophenyl)-1-(methoxycarbonyl)cyclobutyl)ureido)isoquinoline-7-carboxylate according to the same procedure. ¹H NMR (DMSO-d6) δ: 9.66 (s, 1H), 9.03 (s, 1H), 8.98 (d, J=1.6, 1H), 8.71 (s, 1H), 8.30 (dd, J=8.8, 1.6, 1H), 7.91 (d, J=8.8, 1H), 7.52 (d, J=6.5, 1H), 7.44 (m, 2H), 7.35-7.28 (m, 1H), 3.96 (s, 3H), 3.92-3.83 (m, 1H), 3.24-3.07 (m, 2H), 2.67-2.57 (m, 2H); m/z (ES+): 436.1 [M+H⁺]⁺

### 4-(2-(2-Chlorophenyl)-6,8-dioxo-5,7-diazaspiro[3,4]octan-7-yl)isoquinoline-6-carboxylic acid

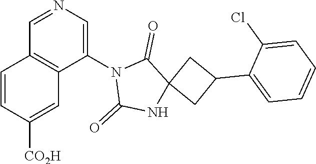 [SMILES: O=C(O)c1ccc2cncc(N3C(=O)NC4(CC(c5ccccc5Cl)C4)C3=O)c2c1]

To a stirred solution of methyl 4-(2-(2-chlorophenyl)-6,8-dioxo-5,7-diazaspiro[3,4]octan-7-yl)isoquinoline-6-carboxylate (2.38 g, 5.46 mmol) in THF (100 mL) in an ice-bath was added lithium hydroxide (13.20 mL, 1M solution in water) and stirring was maintained at 2-5° C. for 48 hours. After concentration under reduced pressure and high vacuum, the resulting yellow foam was treated with DCM (100 mL) and TFA (20 ML) and heated at 65° C. overnight. After cooling to ambient temperature, the reaction mixture was concentrated under reduced pressure and then twice reconcentrated from DCM (50 mL) to give the crude product as a yellow oil. The crude product was partitioned between 2-methyl-THF (250 mL) and water (100 mL) and the biphasic mixture was filtered through diatomaceous earth, the filter cake being well-washed with 2-methyl-THF. The organic phase of the filtrate was separated, and the aqueous component was twice further stirred with 2-methyl-THF (100 mL) for 10 mins before being separated. The combined organic extracts were dried over anhydrous magnesium sulfate and concentrated under reduced pressure to a pale brown foam. Lyophilisation from water (150 mL) and acetonitrile (50 mL) afforded the title compound as a pale brown solid (2.00 g) which was used directly in the next stage without further purification. m/z (ES+): 422.20 [M+H⁺]⁺

Analogously, 4-(2-(2-chlorophenyl)-6,8-dioxo-5,7-diazaspiro[3,4]octan-7-yl)isoquinoline-7-carboxylic acid was prepared from 4-(2-(2-chlorophenyl)-6,8-dioxo-5,7-diazaspiro[3,4]octan-7-yl)isoquinoline-7-carboxylate according to the same procedure.

### Degrader Synthesis C10778-Series—General Procedure

The target degraders were synthesised by treatment of the respective isoquinoline carboxylic acid (1 eq.) with the relevant amine functionalised linker (pre-appended to the E3-ligase ligand) (1-1.1 eq.) in DMF in the presence of HATU (1-1.1 eq.), DMAP (cat.) and DIPEA (2-3 eq.). Upon completion of the reaction, the reaction mixture was concentrated under reduced pressure. The residue was taken up in DCM and washed with 10% aqueous potassium carbonate solution, dried over anhydrous magnesium sulfate and concentrated under reduced pressure. The target compound was typically purified by flash column chromatography and obtained in final form by lyophilisation from water/acetonitrile.

From 4-(2-(2-Chlorophenyl)-6,8-dioxo-5,7-diazaspiro[3,4]octan-7-yl)isoquinoline-6-carboxylic acid was prepared:
C10778L: 4-(2-(2-chlorophenyl)-6,8-dioxo-5,7-diazaspiro[3.4]octan-7-yl)-N-(6-((2-(2,6-dioxopiperidin-3-yl)-1,3-dioxoisoindolin-4-yl)oxy)hexyl)isoquinoline-6-carboxamide
C10778N: 4-(2-(2-chlorophenyl)-6,8-dioxo-5,7-diazaspiro[3.4]octan-7-yl)-N-(8-((2-(2,6-dioxopiperidin-3-yl)-1,3-dioxoisoindolin-4-yl)oxy)octyl)isoquinoline-6-carboxamide
C107780: 4-(2-(2-chlorophenyl)-6,8-dioxo-5,7-diazaspiro[3.4]octan-7-yl)-N-(4-((2-(2,6-dioxopiperidin-3-yl)-1,3-dioxoisoindolin-4-yl)oxy)butyl)isoquinoline-6-carboxamide
C10778P: 4-(2-(2-chlorophenyl)-6,8-dioxo-5,7-diazaspiro[3.4]octan-7-yl)-N-(2-((2-(2,6-dioxopiperidin-3-yl)-1,3-dioxoisoindolin-4-yl)oxy)ethyl)isoquinoline-6-carboxamide
C10778Q: 4-(2-(2-chlorophenyl)-6,8-dioxo-5,7-diazaspiro[3.4]octan-7-yl)-N-(6-((2-(2,6-dioxopiperidin-3-yl)-1,3-dioxoisoindolin-5-yl)amino)hexyl)isoquinoline-6-carboxamide
C10778R: 4-(2-(2-chlorophenyl)-6,8-dioxo-5,7-diazaspiro[3.4]octan-7-yl)-N-(5-(2-(4-(2,6-dioxopiperidin-3-yl)phenoxy)acetamido)pentyl)isoquinoline-6-carboxamide
C10778S: 4-(2-(2-chlorophenyl)-6,8-dioxo-5,7-diazaspiro[3.4]octan-7-yl)-N-(2-(2-(2-(2-((2-(2,6-dioxopiperidin-3-yl)-1,3-dioxoisoindolin-4-yl)oxy)ethoxy)ethoxy)ethoxy)ethyl)isoquinoline-6-carboxamide

From 4-(2-(2-Chlorophenyl)-6,8-dioxo-5,7-diazaspiro[3,4]octan-7-yl)isoquinoline-7-carboxylic acid was prepared:
C10778M: 4-(2-(2-chlorophenyl)-6,8-dioxo-5,7-diazaspiro[3.4]octan-7-yl)-N-(6-((2-(2,6-dioxopiperidin-3-yl)-1,3-dioxoisoindolin-4-yl)oxy)hexyl)isoquinoline-7-carboxamide
   C10778T: 4-(2-(2-chlorophenyl)-6,8-dioxo-5,7-diazaspiro[3.4]octan-7-yl)-N-(8-((2-(2,6-dioxopiperidin-3-yl)-1,3-dioxoisoindolin-4-yl)oxy)octyl)isoquinoline-7-carboxamide
C10778U: 4-(2-(2-chlorophenyl)-6,8-dioxo-5,7-diazaspiro[3.4]octan-7-yl)-N-(4-((2-(2,6-dioxopiperidin-3-yl)-1,3-dioxoisoindolin-4-yl)oxy)butyl)isoquinoline-7-carboxamide
C10778V: 4-(2-(2-chlorophenyl)-6,8-dioxo-5,7-diazaspiro[3.4]octan-7-yl)-N-(2-((2-(2,6-dioxopiperidin-3-yl)-1,3-dioxoisoindolin-4-yl)oxy)ethyl)isoquinoline-7-carboxamide
C10778W: 4-(2-(2-chlorophenyl)-6,8-dioxo-5,7-diazaspiro[3.4]octan-7-yl)-N-(6-((2-(2,6-dioxopiperidin-3-yl)-1,3-dioxoisoindolin-5-yl)amino)hexyl)isoquinoline-7-carboxamide
C10778X: 4-(2-(2-chlorophenyl)-6,8-dioxo-5,7-diazaspiro[3.4]octan-7-yl)-N-(5-(2-(4-(2,6-dioxopiperidin-3-yl)phenoxy)acetamido)pentyl)isoquinoline-7-carboxamide
C10778Y: 4-(2-(2-chlorophenyl)-6,8-dioxo-5,7-diazaspiro[3.4]octan-7-yl)-N-(2-(2-(2-(2-((2-(2,6-dioxopiperidin-3-yl)-1,3-dioxoisoindolin-4-yl)oxy)ethoxy)ethoxy)ethoxy)ethyl)isoquinoline-7-carboxamide

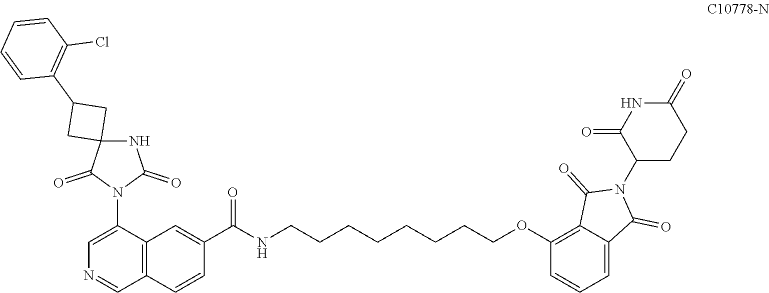 [SMILES: O=C1CCC(N2C(=O)c3cccc(OCCCCCCCCNC(=O)c4ccc5cncc(N6C(=O)NC7(CC(c8ccccc8Cl)C7)C6=O)c5c4)c3C2=O)C(=O)N1]

To a stirred solution of 4-(2-(2-chlorophenyl)-6,8-dioxo-5,7-diazaspiro[3,4]octan-7-yl)isoquinoline-6-carboxylic acid (0.50 g, 1.19 mmol) in DMF (60 mL) at 0° C. was sequentially added HATU (0.45 g, 1-17 mmol), DMAP (0.01 g), thalidomide 4′-ether-alkylC8-amine hydrochloride (0.47 g, 1.07 mmol) and DIPEA (0.35 g, 2.67 mmol). The reaction mixture was stirred at 0° C. for 30 mins and then allowed to warm to ambient temperature with stirring over three hours. The reaction mixture was concentrated under reduced pressure and the residue was dissolved in DCM (50 mL), washed with 10% aqueous potassium carbonate solution (50 mL), dried over anhydrous magnesium sulfate and concentrated under reduced pressure. Purification by flash column chromatography, eluting with 0-5% MeOH/DCM, and subsequent freeze drying from water/MeCN/EtOH (300 mL/100 mL/30 mL) afforded the title compound as a white solid (0.31 g, 33%). ¹H NMR (DMSO-d6) δ: 11.09 (s, 1H), 9.50 (s, 1H), 9.05 (br s, 1H), 8.89 (t, J=5.4, 1H), 8.63 (s, 1H), 8.35 (d, J=8.5, 1H), 8.16-8.04 (m, 2H), 7.81-7.75 (m, 1H), 7.54-7.38 (m, 4H), 7.32-7.25 (m, 1H), 5.07 (dd, J=12.8, 5.4, 1H), 4.14 (t, J=6.3, 2H), 3.92-3.82 (m, 1H), 3.43-3.07 (m, 5H), 2.92-2.82 (m, 1H), 2.72-2.45 (4H), 2.05-1.96 (m, 1H), 1.75-1.65 (m, 2H), 1.60-1.50 (m, 2H), 1.46-1.27 (m, 7H); ¹³C NMR (DMSO-d6) δ: 176.52, 172.82, 169.99, 166.86, 165.41, 165.30, 155.99, 154.21, 153.32, 143.88, 140.57, 137.00, 136.94, 133.24, 132.61, 131.92, 129.27, 129.15, 128.47, 128.26, 127.52, 127.45, 126.36, 124.30, 120.89, 119.73, 116.20, 115.11, 68.76, 57.12, 48.73, 30.96, 28.97, 28.90, 28.74, 28.67, 28.37, 26.46, 25.24, 22.00; m/z (ES+): 805.3 [M+H⁺]⁺

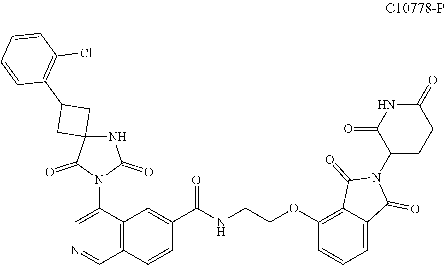 [SMILES: O=C1CCC(N2C(=O)c3cccc(OCCNC(=O)c4ccc5cncc(N6C(=O)NC7(CC(c8ccccc8Cl)C7)C6=O)c5c4)c3C2=O)C(=O)N1]

To a stirred solution of 4-(2-(2-chlorophenyl)-6,8-dioxo-5,7-diazaspiro[3,4]octan-7-yl)isoquinoline-6-carboxylic acid (150 mg, 0.36 mmol) in DMF (20 mL) at 0° C. was sequentially added HATU (115 mg, 0.30 mmol), DMAP (1.6 mg), thalidomide 4′-ether-alkylC2-amine hydrochloride (97 mg, 0.27 mmol) and DIPEA (0.12 mL, 0.69 mmol). The reaction mixture was stirred at 0° C. for 30 mins and then allowed to warm to ambient temperature with stirring for 16 hours. The reaction mixture was concentrated under reduced pressure and the residue was dissolved in DCM (50 mL), washed with 10% aqueous potassium carbonate solution (50 mL), dried over anhydrous magnesium sulfate and concentrated under reduced pressure. Purification by flash column chromatography, eluting with 2-5% MeOH/DCM, and subsequent freeze drying from water/MeCN (30 mL/15 mL) afforded the title compound as a white solid (72 mg, 28%). ¹H NMR (DMSO-d6) δ: 11.08 (s, 1H), 9.51 (s, 1H), 9.17-9.12 (m, 1H), 9.04 (br s, 1H), 8.65 (s, 1H), 8.38 (d, J=90.3, 1H), 8.18-8.14 (m, 2H), 7.82-7.77 (m, 1H), 7.60 (d, J=8.6, 1H), 7.50 (d, J=8.6, 1H), 7.46-7.39 (m, 2H), 7.31 (td, J=70.6, 1.5, 1H), 5.05 (dd, J=12.6, 5.5, 1H), 4.4 (t, J=50.7, 1H), 3.90-3.67 (m, 2H), 3.16-3.08 (m, 1H), 2.91-2.79 (m, 1H), 2.68-2.42 (m, 3H), 2.02-1.94 (m, 1H); ¹³C NMR (DMSO-d6) δ: 176.5, 172.8, 169.9, 166.7, 166.0, 165.2, 155.7, 154.2, 153.4, 144.0, 140.6, 137.0, 136.5, 133.3, 132.6, 132.0, 129.3, 129.2, 128.6, 128.3, 127.5, 127.4, 126.3, 124.4, 121.1, 120.3, 116.7, 115.6, 67.1, 57.1, 48.7, 30.9, 28.9, 22.0; m/z (ES+): 743.2 [M+Na⁺]⁺

### tert-Butyl N-[5-[[2-[4-(2,6-dioxo-3-piperidyl)phenoxy]acetyl]amino]pentyl]carbamate

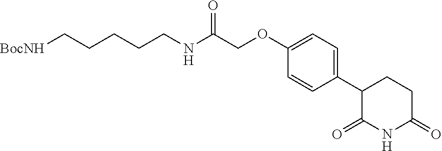 [SMILES: CC(C)(C)OC(=O)NCCCCCNC(=O)COc1ccc(C2CCC(=O)NC2=O)cc1]

To a stirred solution of phenyl glutarimide 4′-oxyacetic acid (0.50 g, 1.90 mmol) and N-Boc-cadaverine (0.38 g, 2.09 mmol) in DMF (20 mL) at 0° C. was sequentially added HATU (0.79 g, 0.34 mmol), DMAP (12 mg) and DIPEA (0.83 mL, 4.75 mmol). The reaction mixture was stirred at 0° C. for 30 mins and then allowed to warm to ambient temperature with stirring for 16 hours. The reaction mixture was concentrated under reduced pressure and the residue was dissolved in DCM (50 mL), washed with 10% aqueous potassium carbonate solution (50 mL), dried over anhydrous magnesium sulfate and concentrated under reduced pressure. Purification by flash column chromatography, eluting with 2-5% MeOH/DCM, afforded the title compound as a white solid (0.84 g, 99%). ¹H NMR (DMSO-d6) δ: 10.79 (s, 1H), 8.04 (t, J=50.7, 1H), 7.14 (d, J=8.7, 2H), 6.91 (d, J=8.7, 2H), 6.75 (t, J=50.2, 1H), 4.44 (s, 2H), 3.79 (dd, J=11.5, 4.9, 1H), 3.14-3.06 (m, 2H), 2.90-2.83 (m, 3H), 2.70-2.60 (m, 2H), 2.50-2.44 (m, 1H), 2.22-2.10 (m, 1H), 2.04-1.96 (m, 1H), 1.45-1.15 (m, 13H); m/z (ES+): 470.10 [M+Na⁺]⁺

### N-(5-Aminopentyl)-2-[4-(2,6-dioxo-3-piperidyl)phenoxy]acetamide hydrochloride

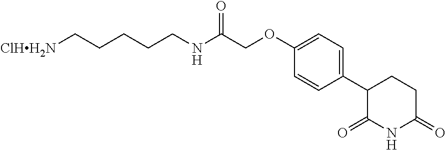

To a stirred solution of tert-butyl N-[5-[[2-[4-(2,6-dioxo-3-piperidyl)phenoxy]acetyl]amino]pentyl]carbamate (0.84 g, 1.88 mmol) in 1,4-dioxane (50 mL) was added hydrogen chloride (4.69 mL, 18.77 mmol, 4M solution in 1,4-dioxane), and the resulting solution was heated at 35° C. for 6 hours. After cooling to ambient temperature, the reaction mixture was concentrated under reduced pressure, and the residue was triturated with diethyl ether (50 mL). The solid precipitate was collected by filtration, the filter-cake being well washed with diethyl ether, and then dried under high vacuum to afford the title compound as a white solid (0.67 g, 90%). ¹H NMR (DMSO-d6) δ: 10.79 (s, 1H), 8.12 (t, J=5.7, 1H), 7.91 (br s, 3H), 7.15 (d, J=8.7, 2H), 6.88 (d, J=8.7, 2H), 4.45 (s, 2H), 3.80 (dd, J=11.5, 4.9, 1H), 3.12 (dd, J=13.1, 6.7, 2H), 2.77-2.60 (m, 3H), 2.50-2.45 (m, 1H), 2.22-2.10 (m, 1H), 2.04-1.96 (m, 1H), 1.60-1.50 (m, 2H), 1.48-1.39 (m, 2H), 1.32-1.23 (m, 2H); m/z (ES+): 348.10 [M_{(freebase)}+H⁺]⁺

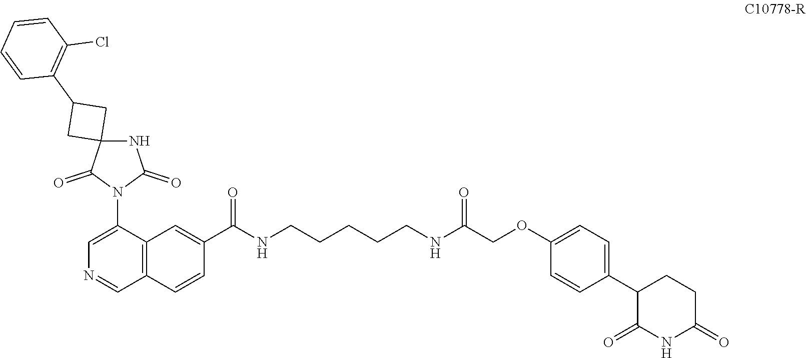 [SMILES: O=C(COc1ccc(C2CCC(=O)NC2=O)cc1)NCCCCCNC(=O)c1ccc2cncc(N3C(=O)NC4(CC(c5ccccc5Cl)C4)C3=O)c2c1]

To a stirred solution of 4-(2-(2-chlorophenyl)-6,8-dioxo-5,7-diazaspiro[3,4]octan-7-yl)isoquinoline-6-carboxylic acid (130 mg, 0.31 mmol) and N-(5-aminopentyl)-2-(4-(2,6-dioxo-3-piperidyl)phenoxy)acetamide hydrochloride (118 mg, 0.31 mmol) in DMF (20 mL) at 0° C. was sequentially added HATU (129 mg, 0.34 mmol), DMAP (0.2 mg) and DIPEA (0.13 mL, 0.77 mmol). The reaction mixture was stirred at 0° C. for 30 mins and then allowed to warm to ambient temperature with stirring for 16 hours. The reaction mixture was concentrated under reduced pressure and the residue was dissolved in DCM (50 mL), washed with 10% aqueous potassium carbonate solution (50 mL), dried over anhydrous magnesium sulfate and concentrated under reduced pressure. Purification by flash column chromatography, eluting with 2-5% MeOH/DCM, and subsequent freeze drying from water/MeCN (50 mL/25 mL) afforded the title compound as a white solid (90 mg, 39%). ¹H NMR (DMSO-d6) δ: 10.79 (s, 1H), 9.51-9.48 (m, 1H), 9.06 (br s, 1H), 8.88 (t, J=5.5, 1H), 8.63 (s, 1H), 8.35 (d, J=8.4, 1H), 8.16-8.12 (m, 2H), 8.05 (t, J=5.8, 1H), 7.52 (d, J=6.7, 1H), 7.46-7.35 (m, 2H), 7.30 (td, J=7.7, 1.6, 1H), 7.13 (d, J=8.7, 1H), 6.89 (d, J=8.7, 1H), 4.43 (s, 2H), 3.93-3.83 (m, 1H), 3.78 (dd, J=11.5, 4.9, 1H), 3.38-3.22 (m, 2H), 3.18-3.08 (m, 4H), 2.72-2.58 (m, 3H), 2.52-2.42 (m, 1H), 2.20-2.09 (m, 1H), 2.03-1.95 (m, 1H), 1.60-1.43 (m, 4H), 1.36-1.27 (m, 2H); ¹³C NMR (DMSO-d6) δ: 176.5, 174.4, 173.4, 167.5, 165.4, 156.7, 154.2, 153.3, 143.9, 140.6, 136.9, 132.6, 132.0, 131.8, 129.6, 129.3, 129.2, 128.5, 128.2, 127.6, 127.5, 126.3, 124.3, 120.9, 114.5, 67.1, 57.1, 46.5, 38.2, 31.4, 28.9, 28.8, 28.7, 26.0, 23.8; m/z (ES+): 751.1 [M+H⁺]⁺

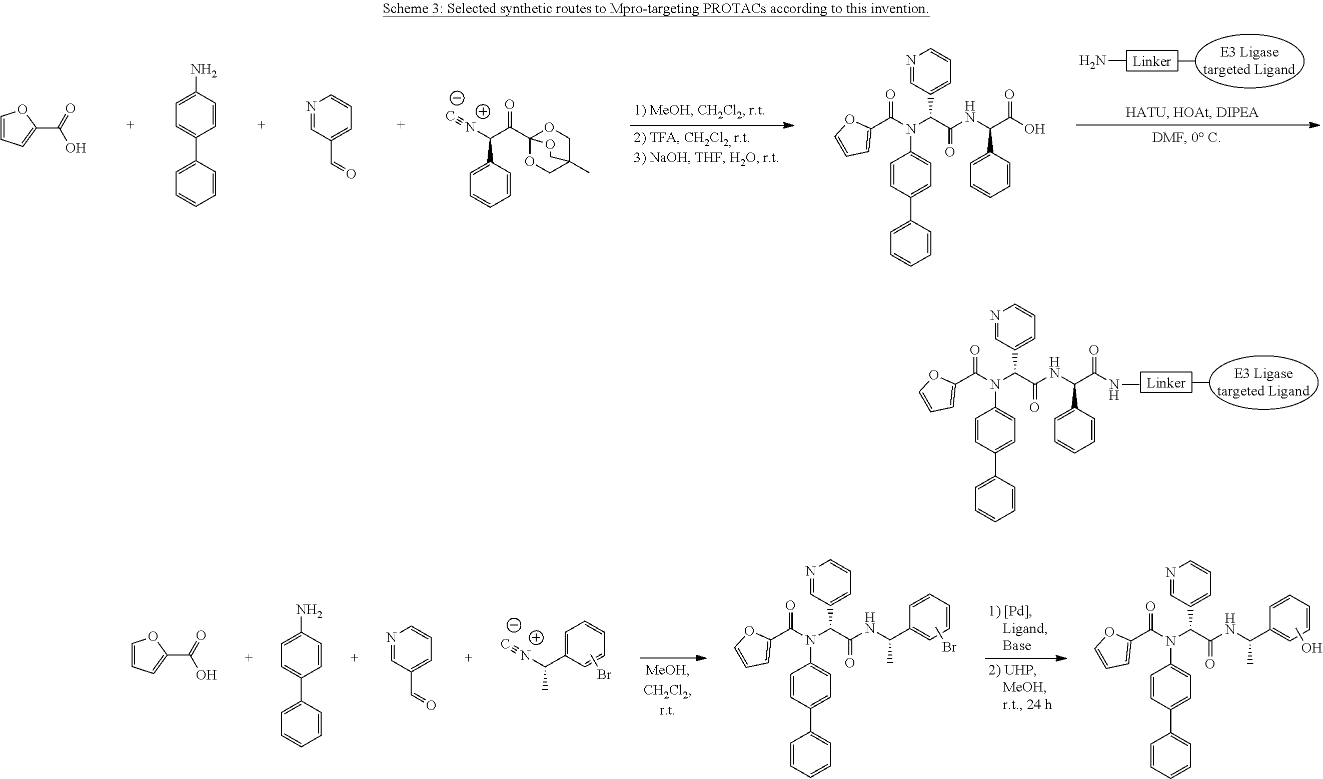

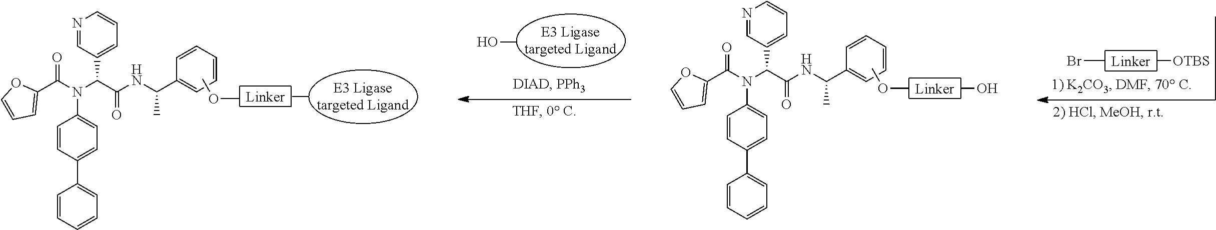

### Inhibitory Concentration (IC50), Antiviral and Plaque Reduction Assays

### Assay Methods

### IC50 Assay

IC₅₀ values for inhibition of the recombinant SARS-CoV-2 main protease were determined by a FRET assay, using the peptide Dabcyl-KTSAVLQ↓SGFRKM-E(Edans)-NH₂ (Biosyntan) as the substrate (↓ indicates the cleavage site). M^{pro} (50 nM) was dissolved in buffer containing 20 mM HEPES, 120 mM NaCl, 0.4 mM EDTA, 20% glycerol, pH 7.0. 4 mM DTT was added just prior to the measurements. The concentration of compounds (in DMSO) was varied between 0 and 100 μM. After incubation of enzyme and compound for 10 minutes at 37° C., the reaction was initiated by adding the FRET substrate at a final concentration of 10 μM to each well (final volume: 100 μL/well, 1% DMSO). A Tecan Spark® fluorescence plate reader was used, with an excitation wavelength of 360 nm and an emission wavelength of 460 nm. GraphPad Prism 9.2.0 software (GraphPad) was used for the calculation of the IC₅₀ values. Measurements of inhibitory activities of the compounds were performed in triplicate and are presented as the mean f SD.

### Antiviral Assay

Vero E6 cells (2×10⁴/well) were seeded in 96 well plates one day before the experiment. Pre-treatment, the medium was removed and compounds were added at concentrations varying from 0.1-100 μM. After 24 h incubation, the medium was removed and cells were infected with MOI 0.05 SARS-CoV-2 SARS-CoV2/ZG/297-20. After 1 h infection, the viruses were removed and the same concentration of compounds were added back into the cells. After 72 h incubation at 37° C., EC₅₀ measurements were made using ATP quantification (Cell Titer Glo).

### Plaque Reduction Assay

Vero E6 cells (5×10⁵/well) were seeded in 12 well plates and pre-treated with compounds one day prior to infection. After 24 h incubation, the medium was removed and cells were infected with MOI 0.1 SARS-CoV-2 SARS-CoV2/ZG/297-20. The same concentrations of compounds were then added back to the cells and incubated for 24 hours. The supernatant was then harvested for the plaque reduction assay, performed as follows: Vero E6 cells were seeded with 80% confluence one day before the experiment in 48 well plates. The supernatant was serially diluted 10-fold in DMEM with 2% FBS. Cells were infected with the diluted supernatant at 37° C. for 1 h. After incubation, the supernatant was removed and the cells were washed with PBS once. 1.5% overlay (1:1 3% CMC and 2×DMEM) was added prior to incubation at 37° C. for 4-5 days. Cells were fixed with 4% formalin and stained with crystal violet.”

### Assay Results

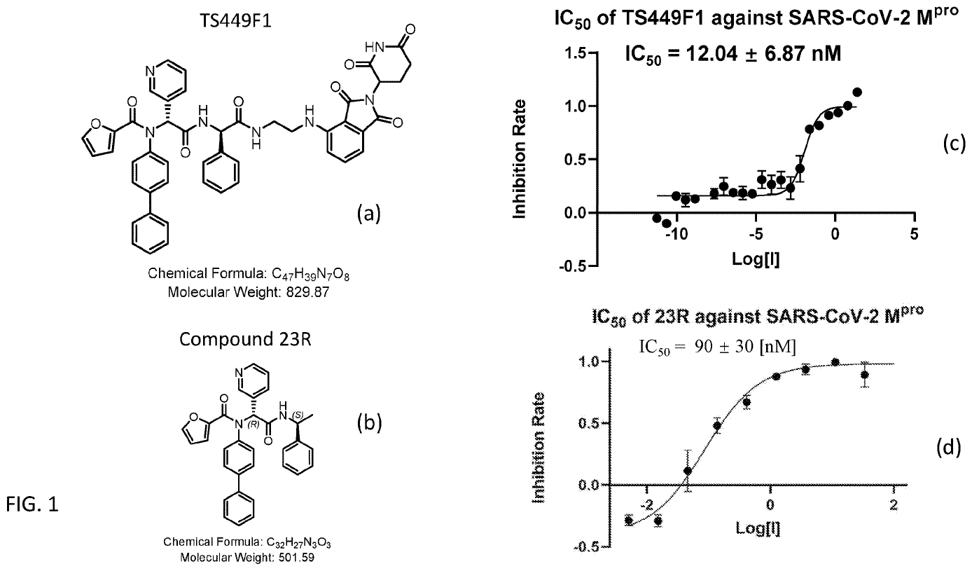

FIG. 1 shows results of an example of a PROTAC (TS449F1) and its parent inhibitor (compound 23R) with inhibitory potency against SARS-CoV-2 Mpro in a binary IC50 experiment. IC50 values have been determined in an in vitro inhibitory assay whereby the fluorescence signal resulting from cleavage of a fluorogenic substrate by Mpro is measured. Inhibition of Mpro is associated with reduced cleavage of the fluorogenic substrate. The PROTAC TS449F1 retains nM activity for Mpro, similar to the parent compound 23R, thus validating the amide exit vector used on the targeting ligand. Assay performed in 20 mM HEPES pH 7.0, 120 mM NaCl, 4 mM DTT, 0.4 mM EDTA and 20% Glycerol. Final DMSO concentration is 1%. Inhibition rate was determined by cleavage of the fluorogenic substrate Dabcyl-KTSAVLQSGFRKM-E(Edans)-Amide (10 μM).

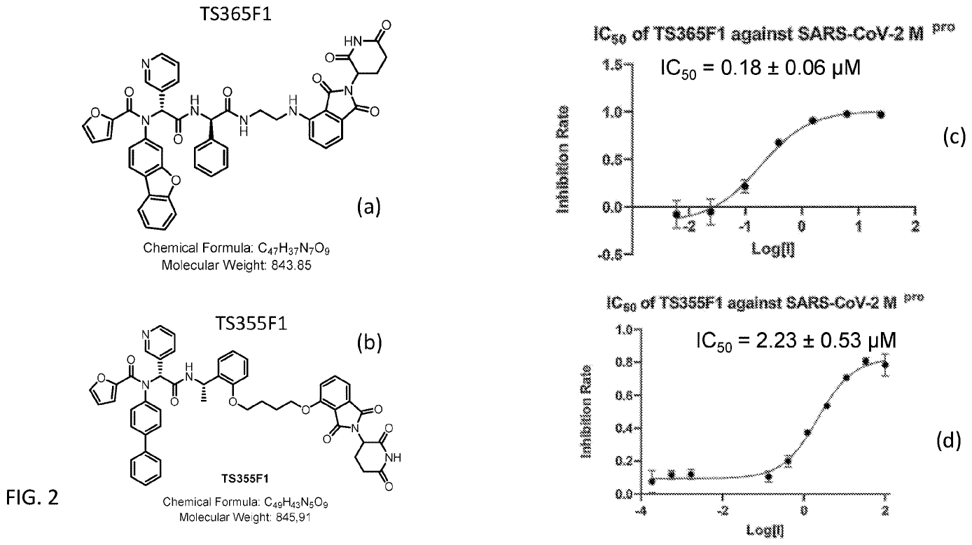

FIG. 2 shows further examples of PROTAC structures that inhibit SARS-COV-2 Mpro. Reaction conditions are the same as described for FIG. 1. TS365F1 uses a dibenzofuran-containing analogue of 23R analogue as the targeting ligand. TS355F1 provides a further example based on an analogue of 23R as the targeting ligand. Both compounds retain inhibitory activity against Mpro and validate the targeting ligand analog and exit vector.

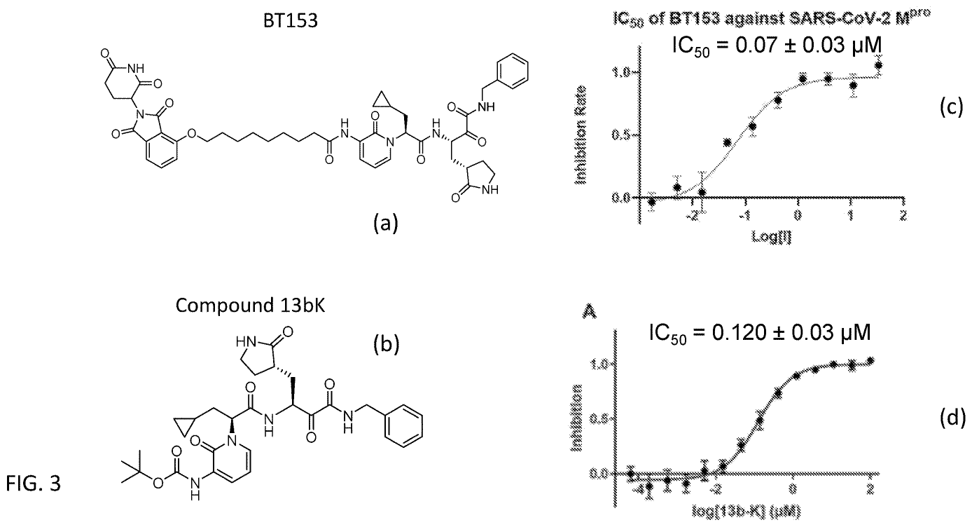

FIG. 3 shows a further example of binary target engagement from a PROTAC structure (BT153) based on a different targeting ligand, 13bK. An in vitro inhibitory assay (described for FIG. 1) was performed for both compounds (FIG. 3c-d). The results demonstrate that BT153 retains similar inhibitory activity for Mpro as the parent inhibitor, 13b-K, thus validating the exit vector.

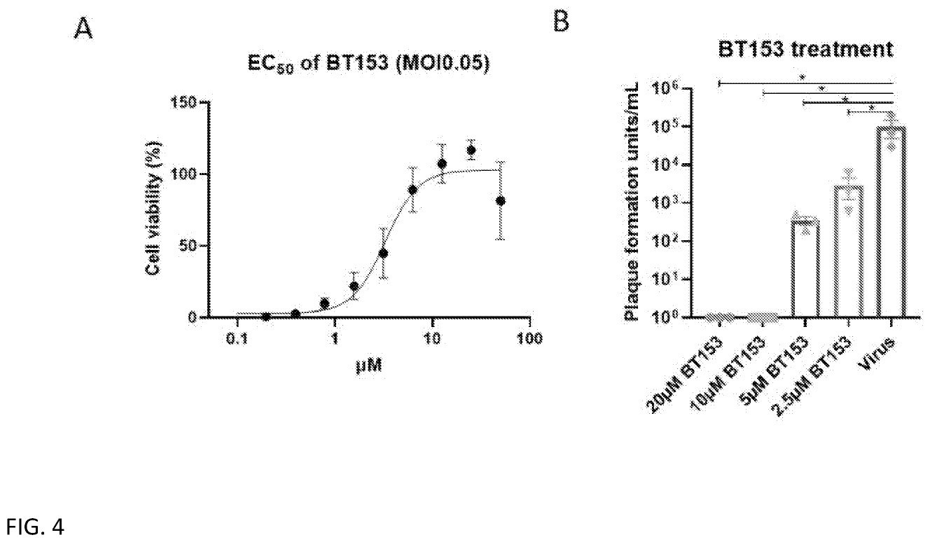

FIG. 4 shows in vitro biology of PROTACs. (A). BT153 shows an EC50 of 3.2 μM in an antiviral assay with VeroE6 infected with SARS-CoV-2 at an MOI of 0.05. (B). A plaque reduction assay performed in Vero E6 cells with BT153 demonstrates efficient reduction of viable virus.

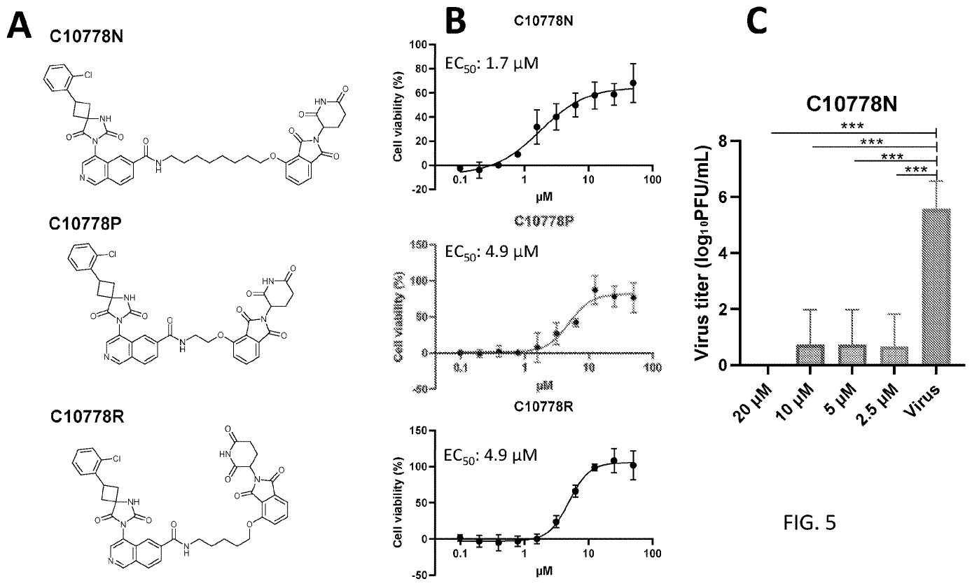

FIG. 5 shows in vitro biology of PROTACs. (A) Chemical structures of selected C10778-series PROTACs, C10778N, C10778P, C10778R. (B) Antiviral assays with Vero E6 cells infected with SARS-CoV-2 at an MOI of 0.05, following dosing with the compounds shown in (A). EC₅₀ values range from 1.7 μM (C10778N) to 4.9 μM (C10778P and C10778R). (C) A plaque reduction assay performed in Vero E6 cells demonstrating that C10778N efficiently reduces viable virus.

Further results are set out in Table 3, below. It should be noted that the assay results in the Figures and Table 3 were obtained under the conditions tested and are indicative but are not exhaustive. The performance of the compounds under other conditions may be even better.

| | | | Antiviral | |
|---|---|---|---|---|
| Compound | | | Activity | |
| Name | Structure | IC50 | 50 uM | 10 uM |
| BT150 | 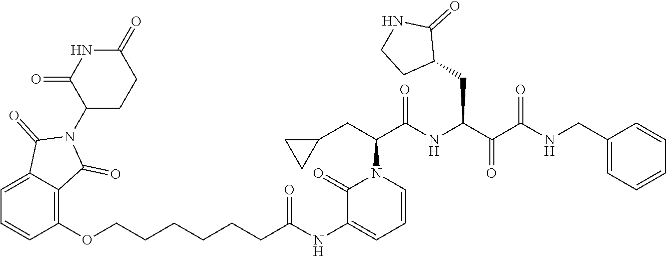 [SMILES: O=C1CCC(N2C(=O)c3cccc(OCCCCCCC(=O)Nc4cccn([C@@H](CC5CC5)C(=O)N[C@@H](C[C@@H]5CCNC5=O)C(=O)C(=O)NCc5ccccc5)c4=O)c3C2=O)C(=O)N1] | B | D | D |
| BT152 | 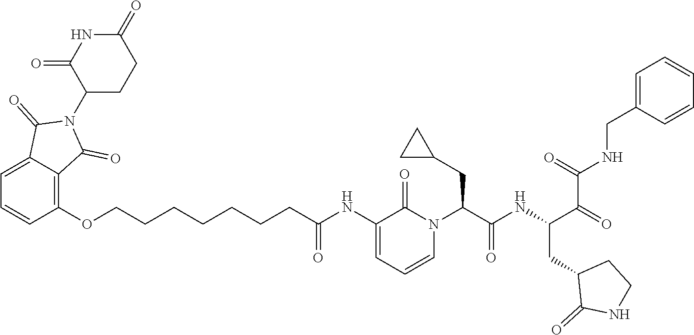 [SMILES: O=C1CCC(N2C(=O)c3cccc(OCCCCCCCC(=O)Nc4cccn([C@@H](CC5CC5)C(=O)N[C@@H](C[C@@H]5CCNC5=O)C(=O)C(=O)NCc5ccccc5)c4=O)c3C2=O)C(=O)N1] | B | C | D |
| BT153 | 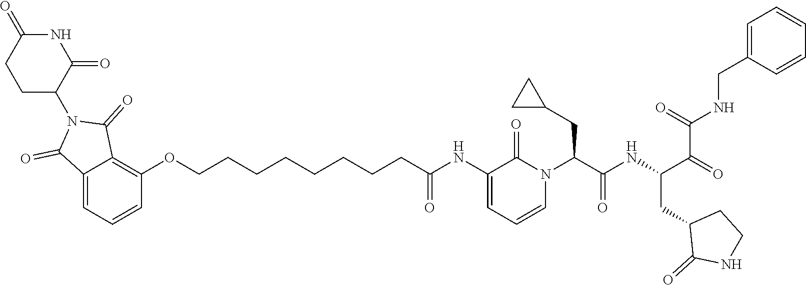 [SMILES: O=C1CCC(N2C(=O)c3cccc(OCCCCCCCCC(=O)Nc4cccn([C@@H](CC5CC5)C(=O)N[C@@H](C[C@@H]5CCNC5=O)C(=O)C(=O)NCc5ccccc5)c4=O)c3C2=O)C(=O)N1] | A | B | B |
| C10778L | 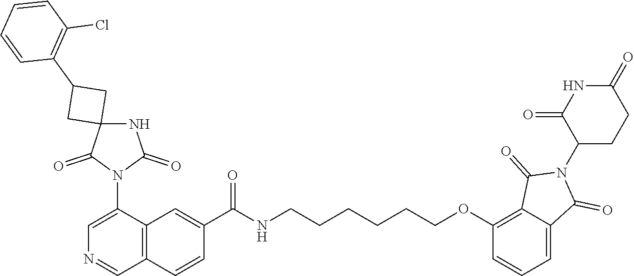 [SMILES: O=C1CCC(N2C(=O)c3cccc(OCCCCCCNC(=O)c4ccc5cncc(N6C(=O)NC7(CC(c8ccccc8Cl)C7)C6=O)c5c4)c3C2=O)C(=O)N1] | A | D | D |
| C10778M | 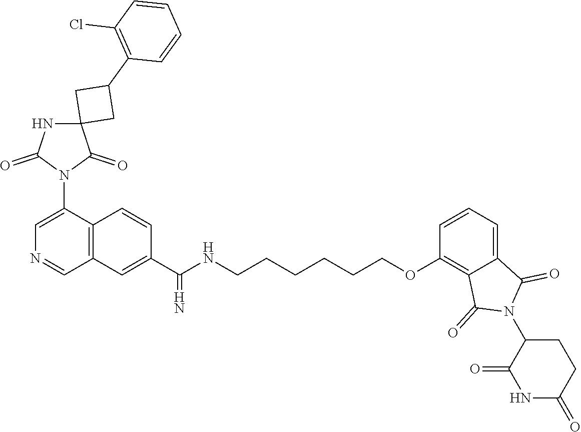 [SMILES: N=C(NCCCCCCOc1cccc2c1C(=O)N(C1CCC(=O)NC1=O)C2=O)c1ccc2c(N3C(=O)NC4(CC(c5ccccc5Cl)C4)C3=O)cncc2c1] | B | E | E |
| C10778N | 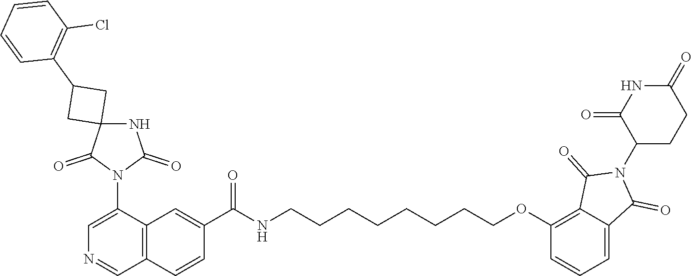 [SMILES: O=C1CCC(N2C(=O)c3cccc(OCCCCCCCCNC(=O)c4ccc5cncc(N6C(=O)NC7(CC(c8ccccc8Cl)C7)C6=O)c5c4)c3C2=O)C(=O)N1] | A | A | B |
| C107780 | 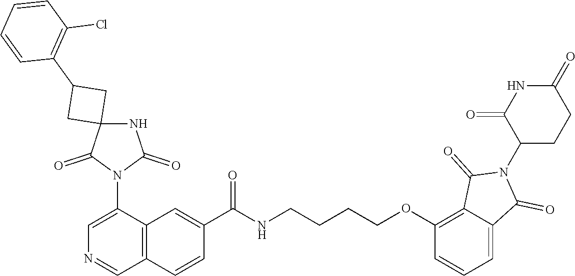 [SMILES: O=C1CCC(N2C(=O)c3cccc(OCCCCNC(=O)c4ccc5cncc(N6C(=O)NC7(CC(c8ccccc8Cl)C7)C6=O)c5c4)c3C2=O)C(=O)N1] | A | A | D |
| C10778P | 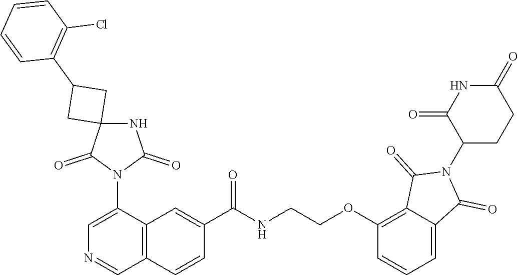 [SMILES: O=C1CCC(N2C(=O)c3cccc(OCCNC(=O)c4ccc5cncc(N6C(=O)NC7(CC(c8ccccc8Cl)C7)C6=O)c5c4)c3C2=O)C(=O)N1] | A | A | A |
| C10778Q | 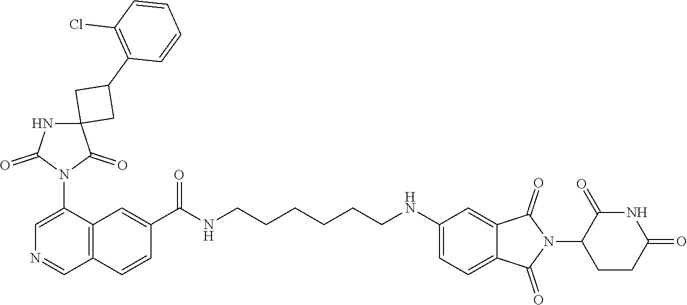 [SMILES: O=C1CCC(N2C(=O)c3ccc(NCCCCCCNC(=O)c4ccc5cncc(N6C(=O)NC7(CC(c8ccccc8Cl)C7)C6=O)c5c4)cc3C2=O)C(=O)N1] | B | D | E |
| C10778R | 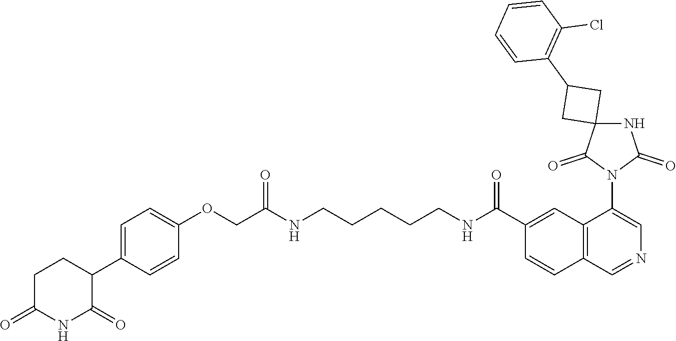 [SMILES: O=C(COc1ccc(C2CCC(=O)NC2=O)cc1)NCCCCCNC(=O)c1ccc2cncc(N3C(=O)NC4(CC(c5ccccc5Cl)C4)C3=O)c2c1] | A | A | A |
| C10778S | 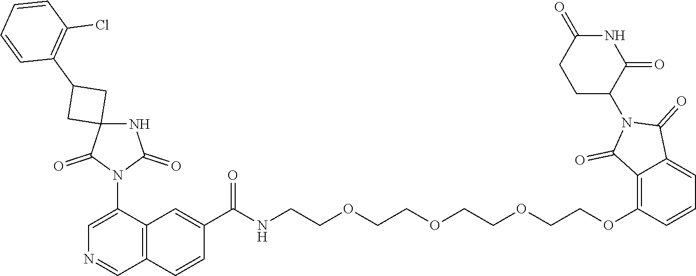 [SMILES: O=C1CCC(N2C(=O)c3cccc(OCCOCCOCCOCCNC(=O)c4ccc5cncc(N6C(=O)NC7(CC(c8ccccc8Cl)C7)C6=O)c5c4)c3C2=O)C(=O)N1] | A | A | D |
| C10778T | 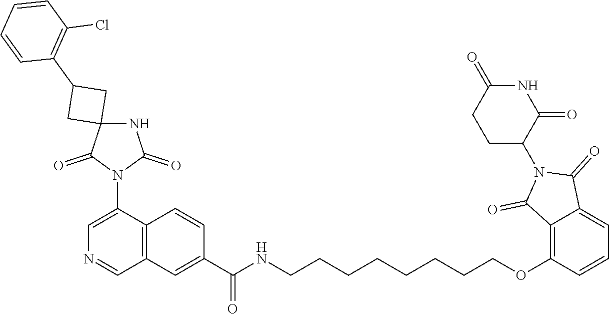 [SMILES: O=C1CCC(N2C(=O)c3cccc(OCCCCCCCCNC(=O)c4ccc5c(N6C(=O)NC7(CC(c8ccccc8Cl)C7)C6=O)cncc5c4)c3C2=O)C(=O)N1] | A | E | E |
| C10778U | 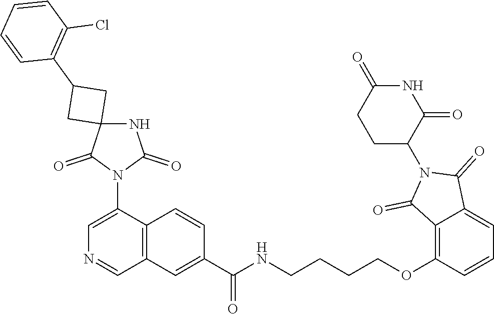 [SMILES: O=C1CCC(N2C(=O)c3cccc(OCCCCNC(=O)c4ccc5c(N6C(=O)NC7(CC(c8ccccc8Cl)C7)C6=O)cncc5c4)c3C2=O)C(=O)N1] | A | D | D |
| C10778V | 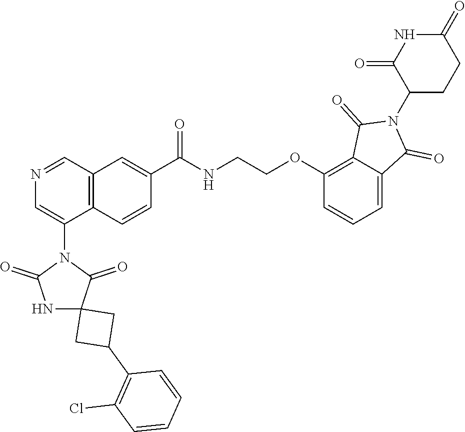 [SMILES: O=C1CCC(N2C(=O)c3cccc(OCCNC(=O)c4ccc5c(N6C(=O)NC7(CC(c8ccccc8Cl)C7)C6=O)cncc5c4)c3C2=O)C(=O)N1] | A | D | D |
| C10778W | 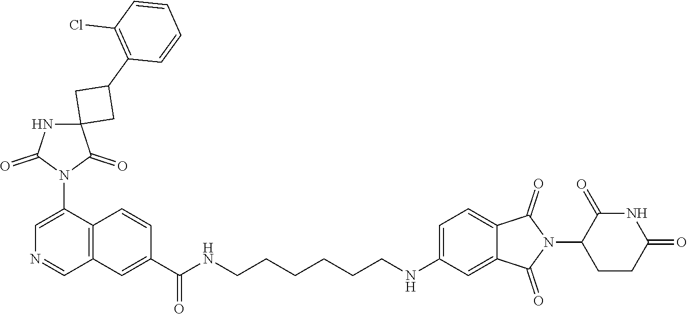 [SMILES: O=C1CCC(N2C(=O)c3ccc(NCCCCCCNC(=O)c4ccc5c(N6C(=O)NC7(CC(c8ccccc8Cl)C7)C6=O)cncc5c4)cc3C2=O)C(=O)N1] | B | E | E |
| C10778X | 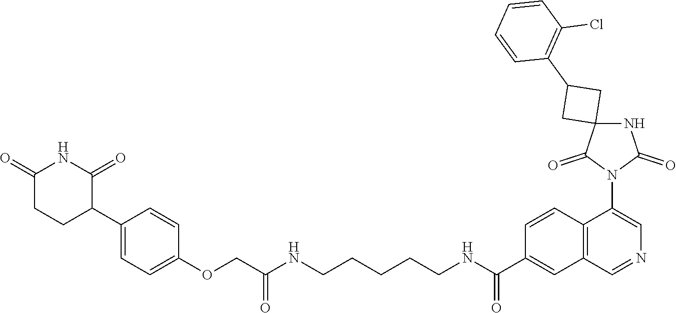 [SMILES: O=C(COc1ccc(C2CCC(=O)NC2=O)cc1)NCCCCCNC(=O)c1ccc2c(N3C(=O)NC4(CC(c5ccccc5Cl)C4)C3=O)cncc2c1] | A | E | E |
| C10778Y | 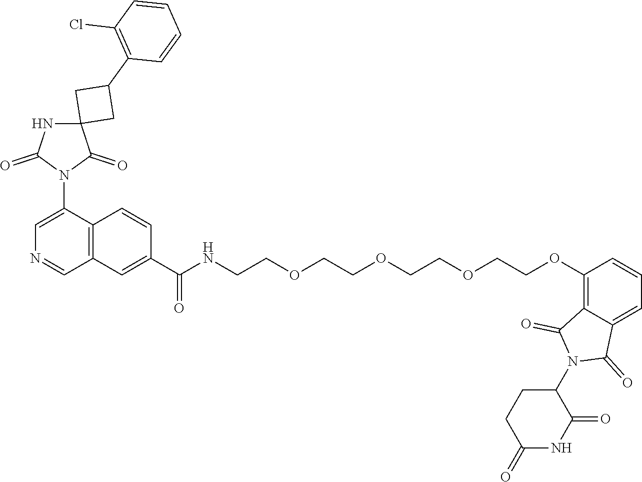 [SMILES: O=C1CCC(N2C(=O)c3cccc(OCCOCCOCCOCCNC(=O)c4ccc5c(N6C(=O)NC7(CC(c8ccccc8Cl)C7)C6=O)cncc5c4)c3C2=O)C(=O)N1] | A | C | D |
| TS325F1 | 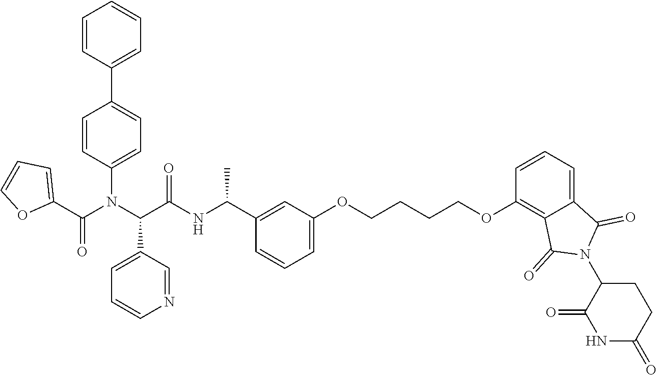 | C | F | F |
| TS325F2 | 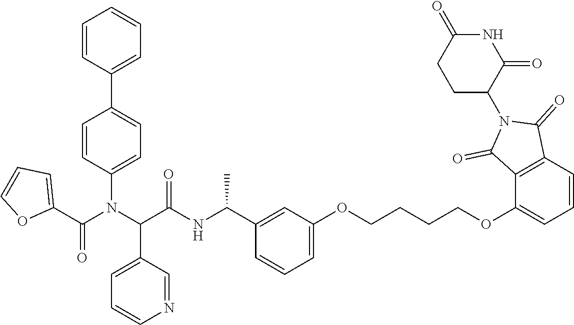 | C | F | F |
| TS338F1 | 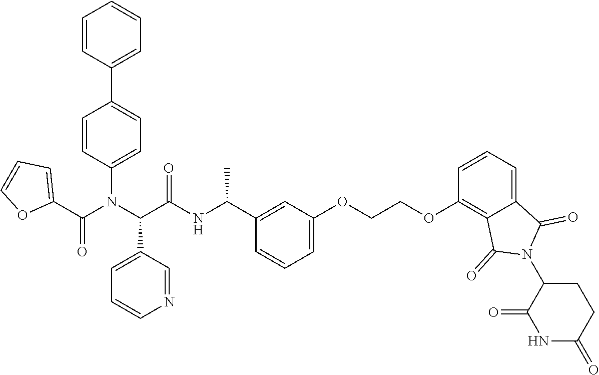 | D | F | F |
| TS339F2 | 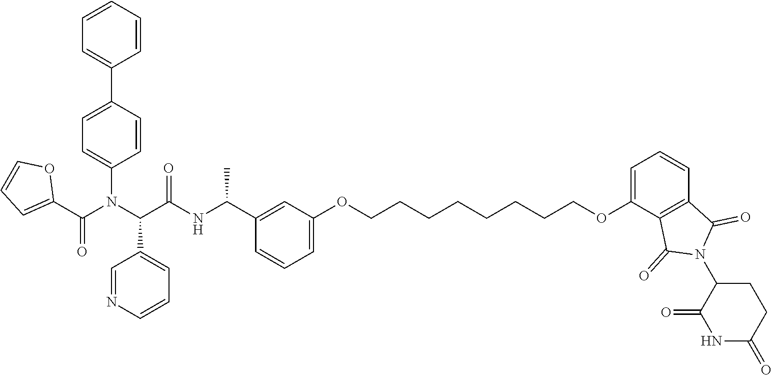 | D | F | F |
| TS355F1 | 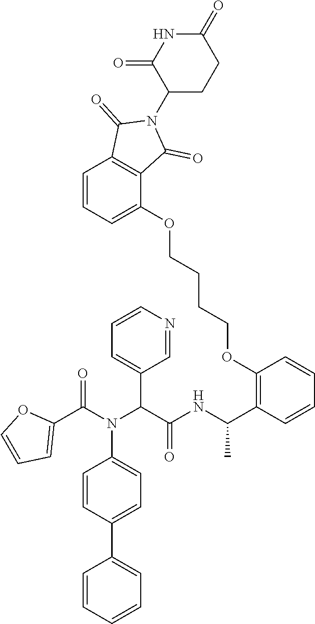 | C | F | F |
| TS449F1 | 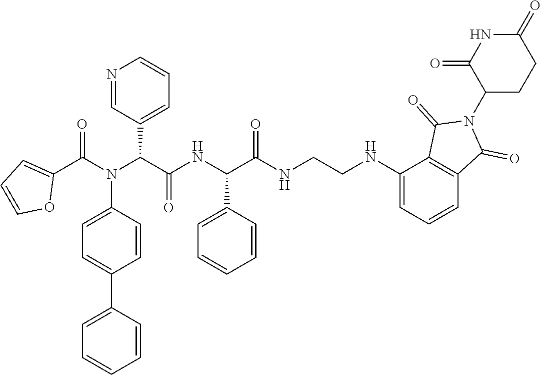 [SMILES: O=C1CCC(N2C(=O)c3cccc(NCCNC(=O)[C@@H](NC(=O)[C@@H](c4cccnc4)N(C(=O)c4ccco4)c4ccc(-c5ccccc5)cc4)c4ccccc4)c3C2=O)C(=O)N1] | A | E | E |
| TS450F1 | 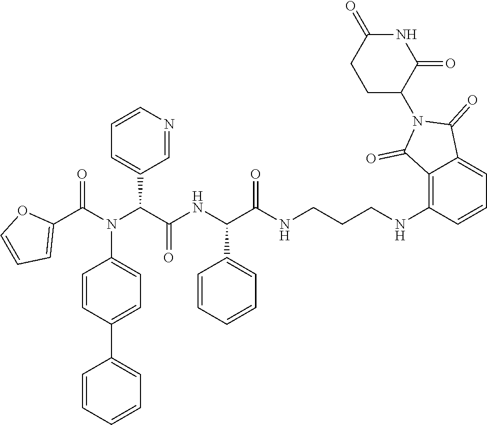 [SMILES: O=C1CCC(N2C(=O)c3cccc(NCCCNC(=O)[C@@H](NC(=O)[C@@H](c4cccnc4)N(C(=O)c4ccco4)c4ccc(-c5ccccc5)cc4)c4ccccc4)c3C2=O)C(=O)N1] | B | E | E |
| TS451F1 | 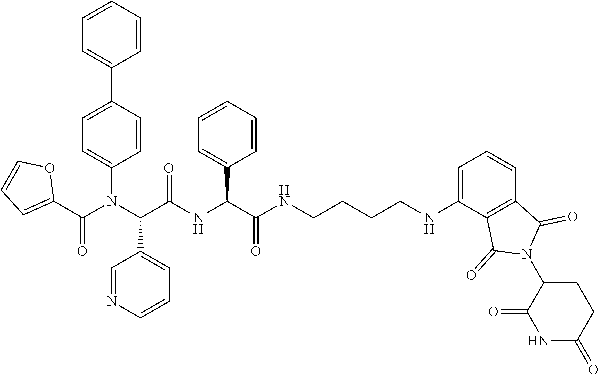 [SMILES: O=C1CCC(N2C(=O)c3cccc(NCCCCNC(=O)[C@@H](NC(=O)[C@H](c4cccnc4)N(C(=O)c4ccco4)c4ccc(-c5ccccc5)cc4)c4ccccc4)c3C2=O)C(=O)N1] | C | E | E |
| TS453F1b | 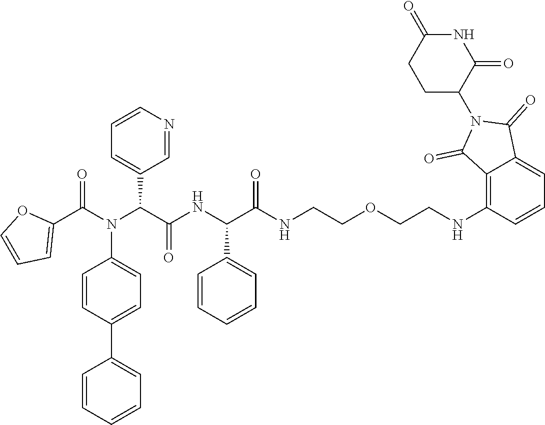 [SMILES: O=C1CCC(N2C(=O)c3cccc(NCCOCCNC(=O)[C@@H](NC(=O)[C@@H](c4cccnc4)N(C(=O)c4ccco4)c4ccc(-c5ccccc5)cc4)c4ccccc4)c3C2=O)C(=O)N1] | B | E | E |
| TS454F1 | 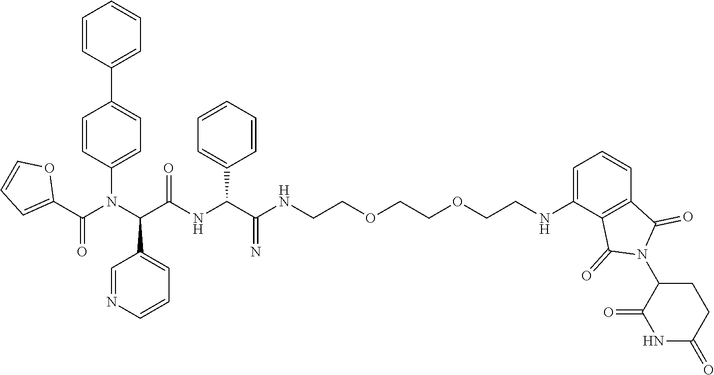 [SMILES: N=C(NCCOCCOCCNc1cccc2c1C(=O)N(C1CCC(=O)NC1=O)C2=O)[C@H](NC(=O)[C@@H](c1cccnc1)N(C(=O)c1ccco1)c1ccc(-c2ccccc2)cc1)c1ccccc1] | B | E | E |
| TS455F1 | 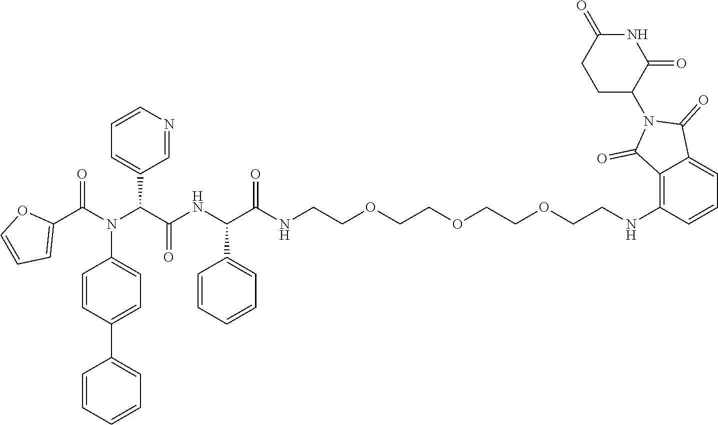 [SMILES: O=C1CCC(N2C(=O)c3cccc(NCCOCCOCCOCCNC(=O)[C@@H](NC(=O)[C@@H](c4cccnc4)N(C(=O)c4ccco4)c4ccc(-c5ccccc5)cc4)c4ccccc4)c3C2=O)C(=O)N1] | A | E | E |
| TS456F1 | 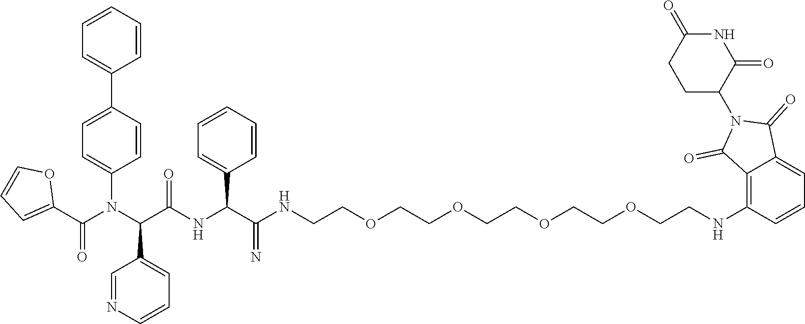 [SMILES: N=C(NCCOCCOCCOCCOCCNc1cccc2c1C(=O)N(C1CCC(=O)NC1=O)C2=O)[C@@H](NC(=O)[C@@H](c1cccnc1)N(C(=O)c1ccco1)c1ccc(-c2ccccc2)cc1)c1ccccc1] | B | E | E |
| TS457F1 | 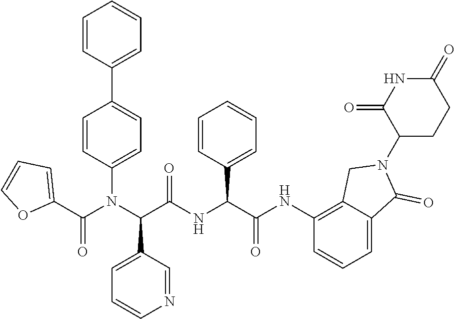 | B | F | F |
| TS462 | 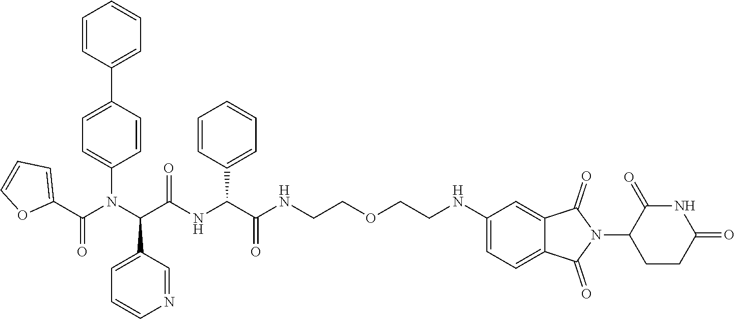 [SMILES: O=C1CCC(N2C(=O)c3ccc(NCCOCCNC(=O)[C@H](NC(=O)[C@@H](c4cccnc4)N(C(=O)c4ccco4)c4ccc(-c5ccccc5)cc4)c4ccccc4)cc3C2=O)C(=O)N1] | B | D | D |
| TS464 | 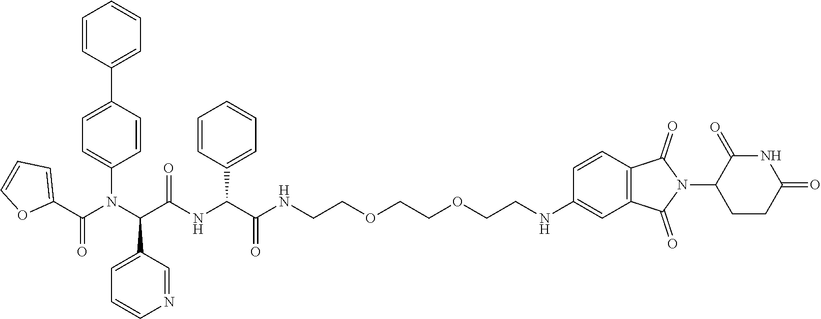 [SMILES: O=C1CCC(N2C(=O)c3ccc(NCCOCCOCCNC(=O)[C@H](NC(=O)[C@@H](c4cccnc4)N(C(=O)c4ccco4)c4ccc(-c5ccccc5)cc4)c4ccccc4)cc3C2=O)C(=O)N1] | B | D | E |
| TS465 | 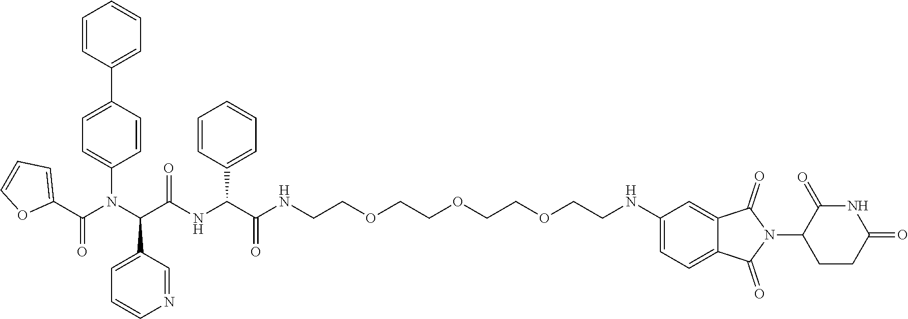 [SMILES: O=C1CCC(N2C(=O)c3ccc(NCCOCCOCCOCCNC(=O)[C@H](NC(=O)[C@@H](c4cccnc4)N(C(=O)c4ccco4)c4ccc(-c5ccccc5)cc4)c4ccccc4)cc3C2=O)C(=O)N1] | A | C | D |
| TS466 | 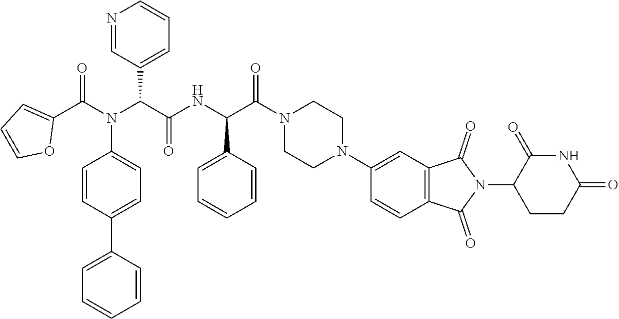 [SMILES: O=C1CCC(N2C(=O)c3ccc(N4CCN(C(=O)[C@H](NC(=O)[C@@H](c5cccnc5)N(C(=O)c5ccco5)c5ccc(-c6ccccc6)cc5)c5ccccc5)CC4)cc3C2=O)C(=O)N1] | A | E | E |
| TS467 | 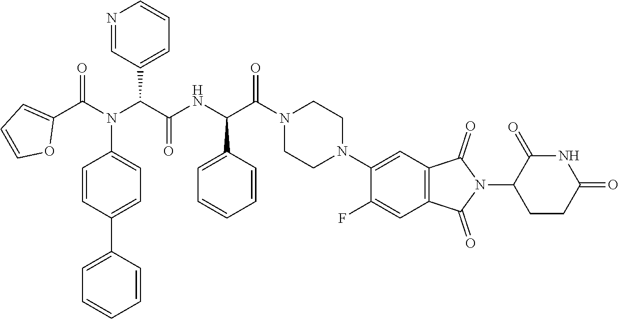 [SMILES: O=C1CCC(N2C(=O)c3cc(F)c(N4CCN(C(=O)[C@H](NC(=O)[C@@H](c5cccnc5)N(C(=O)c5ccco5)c5ccc(-c6ccccc6)cc5)c5ccccc5)CC4)cc3C2=O)C(=O)N1] | A | D | D |
| TS470 | 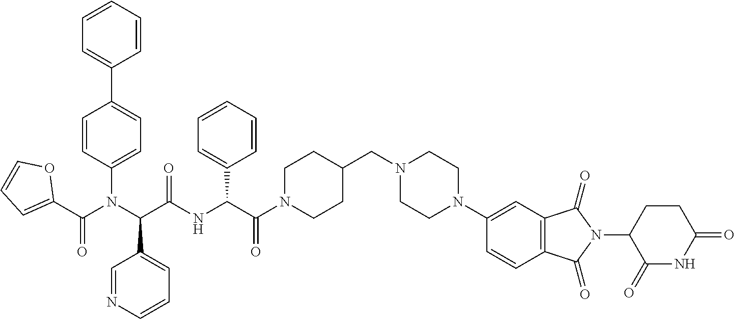 [SMILES: O=C1CCC(N2C(=O)c3ccc(N4CCN(CC5CCN(C(=O)[C@H](NC(=O)[C@@H](c6cccnc6)N(C(=O)c6ccco6)c6ccc(-c7ccccc7)cc6)c6ccccc6)CC5)CC4)cc3C2=O)C(=O)N1] | A | C | D |
| TS471 | 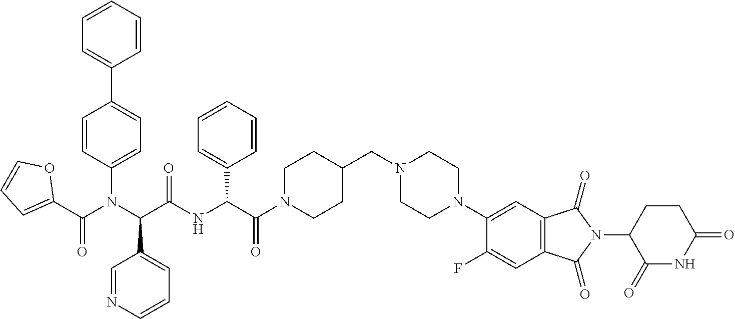 [SMILES: O=C1CCC(N2C(=O)c3cc(F)c(N4CCN(CC5CCN(C(=O)[C@H](NC(=O)[C@@H](c6cccnc6)N(C(=O)c6ccco6)c6ccc(-c7ccccc7)cc6)c6ccccc6)CC5)CC4)cc3C2=O)C(=O)N1] | B | D | D |
| TS476 | 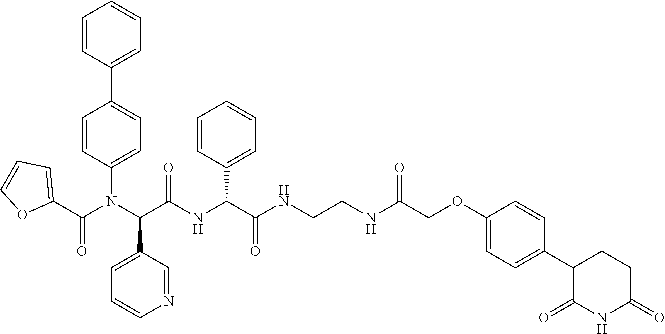 [SMILES: O=C(COc1ccc(C2CCC(=O)NC2=O)cc1)NCCNC(=O)[C@H](NC(=O)[C@@H](c1cccnc1)N(C(=O)c1ccco1)c1ccc(-c2ccccc2)cc1)c1ccccc1] | A | E | E |
| TS477 | 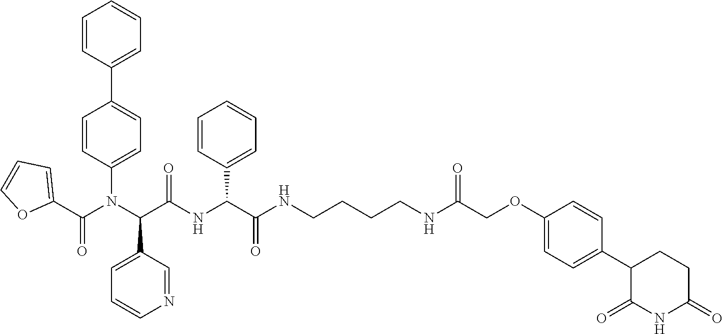 [SMILES: O=C(COc1ccc(C2CCC(=O)NC2=O)cc1)NCCCCNC(=O)[C@H](NC(=O)[C@@H](c1cccnc1)N(C(=O)c1ccco1)c1ccc(-c2ccccc2)cc1)c1ccccc1] | A | E | E |
| TS284F2 | 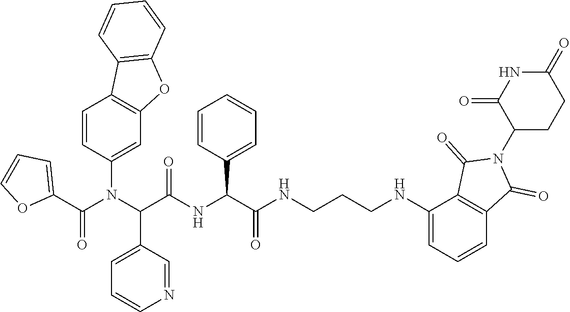 [SMILES: O=C1CCC(N2C(=O)c3cccc(NCCCNC(=O)[C@@H](NC(=O)C(c4cccnc4)N(C(=O)c4ccco4)c4ccc5c(c4)oc4ccccc45)c4ccccc4)c3C2=O)C(=O)N1] | C | E | E |
| TS296 | 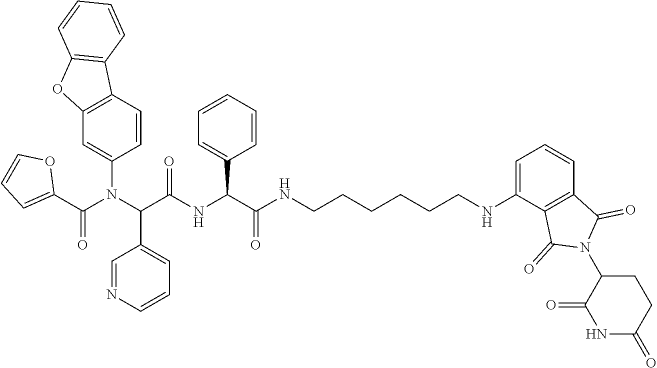 [SMILES: O=C1CCC(N2C(=O)c3cccc(NCCCCCCNC(=O)[C@@H](NC(=O)C(c4cccnc4)N(C(=O)c4ccco4)c4ccc5c(c4)oc4ccccc45)c4ccccc4)c3C2=O)C(=O)N1] | E | E | E |
| TS297F1 | 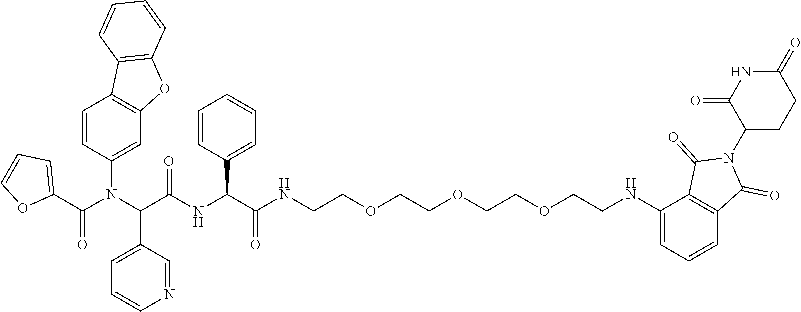 [SMILES: O=C1CCC(N2C(=O)c3cccc(NCCOCCOCCOCCNC(=O)[C@@H](NC(=O)C(c4cccnc4)N(C(=O)c4ccco4)c4ccc5c(c4)oc4ccccc45)c4ccccc4)c3C2=O)C(=O)N1] | B | E | E |
| TS301F1 | 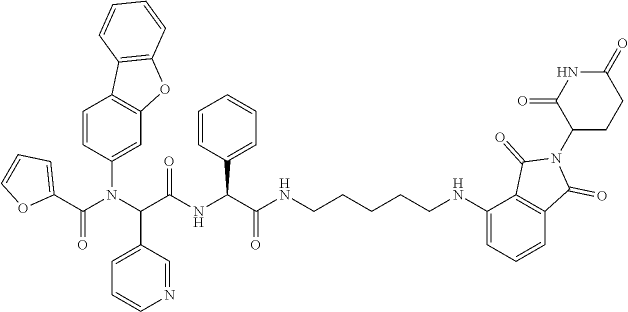 [SMILES: O=C1CCC(N2C(=O)c3cccc(NCCCCCNC(=O)[C@@H](NC(=O)C(c4cccnc4)N(C(=O)c4ccco4)c4ccc5c(c4)oc4ccccc45)c4ccccc4)c3C2=O)C(=O)N1] | D | E | E |
| TS302F1 | 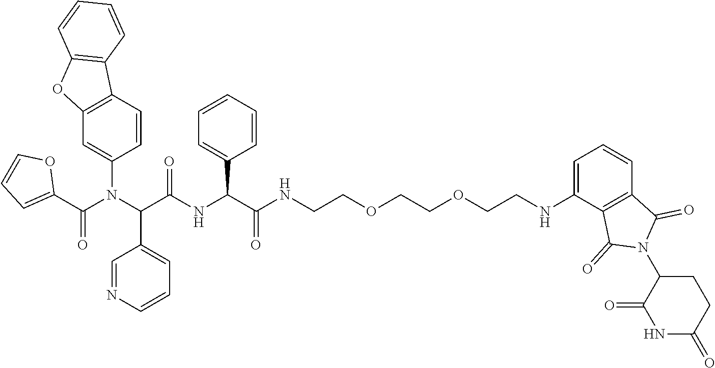 | C | F | F |
| TS303F1 | 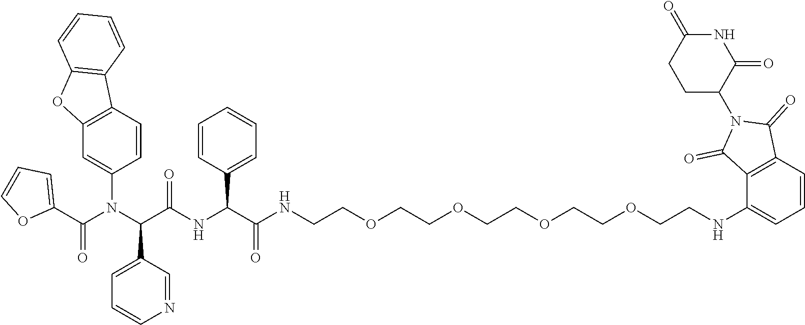 [SMILES: O=C1CCC(N2C(=O)c3cccc(NCCOCCOCCOCCOCCNC(=O)[C@@H](NC(=O)[C@@H](c4cccnc4)N(C(=O)c4ccco4)c4ccc5c(c4)oc4ccccc45)c4ccccc4)c3C2=O)C(=O)N1] | B | E | E |
| TS312F1 | 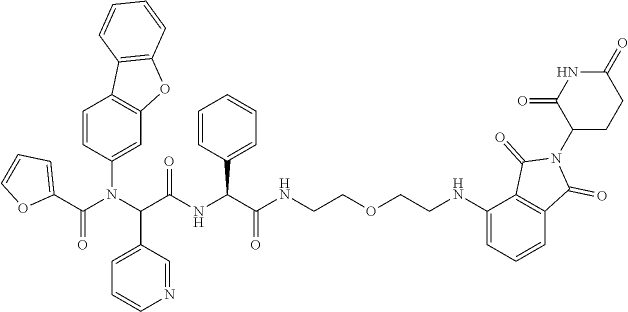 [SMILES: O=C1CCC(N2C(=O)c3cccc(NCCOCCNC(=O)[C@@H](NC(=O)C(c4cccnc4)N(C(=O)c4ccco4)c4ccc5c(c4)oc4ccccc45)c4ccccc4)c3C2=O)C(=O)N1] | C | E | E |
| TS313F1 | 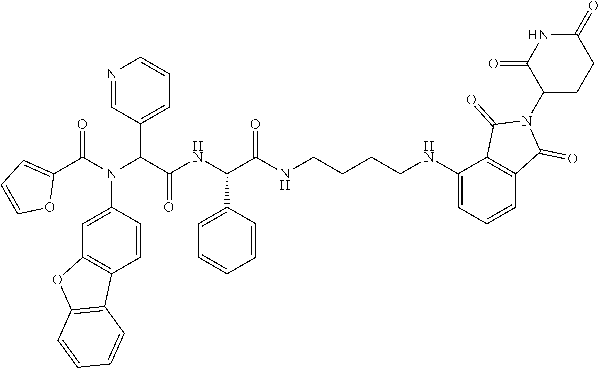 [SMILES: O=C1CCC(N2C(=O)c3cccc(NCCCCNC(=O)[C@@H](NC(=O)C(c4cccnc4)N(C(=O)c4ccco4)c4ccc5c(c4)oc4ccccc45)c4ccccc4)c3C2=O)C(=O)N1] | B | E | D |
| TS347F1 | 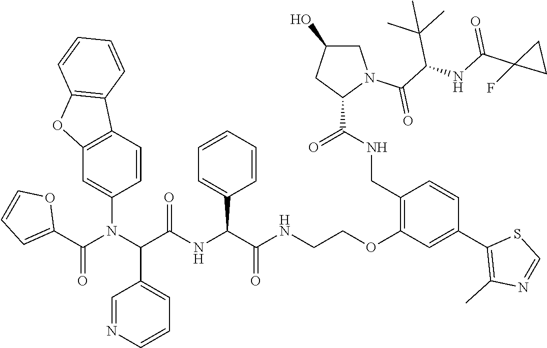 | C | F | F |
| TS349F1 | 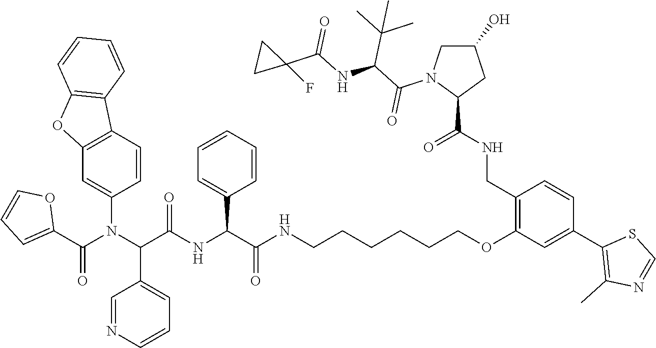 | C | F | F |
| TS365F1 | 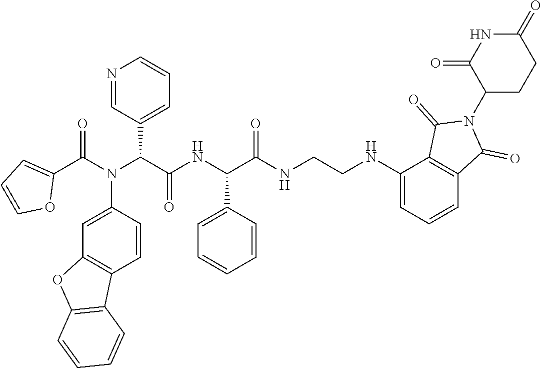 [SMILES: O=C1CCC(N2C(=O)c3cccc(NCCNC(=O)[C@@H](NC(=O)[C@@H](c4cccnc4)N(C(=O)c4ccco4)c4ccc5c(c4)oc4ccccc45)c4ccccc4)c3C2=O)C(=O)N1] | B | D | B |
| TS369F1 | 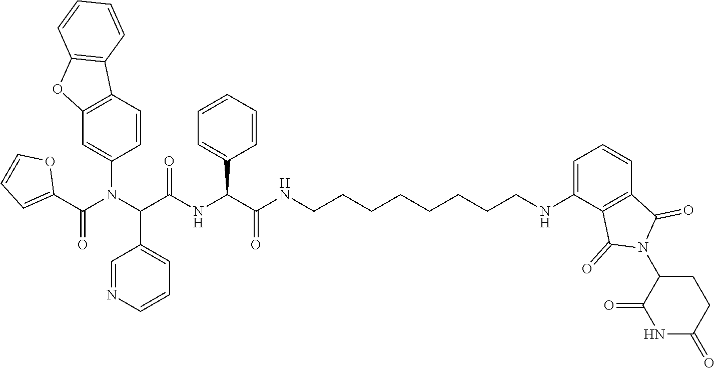 [SMILES: O=C1CCC(N2C(=O)c3cccc(NCCCCCCCCNC(=O)[C@@H](NC(=O)C(c4cccnc4)N(C(=O)c4ccco4)c4ccc5c(c4)oc4ccccc45)c4ccccc4)c3C2=O)C(=O)N1] | E | D | D |
| TS370F1 | 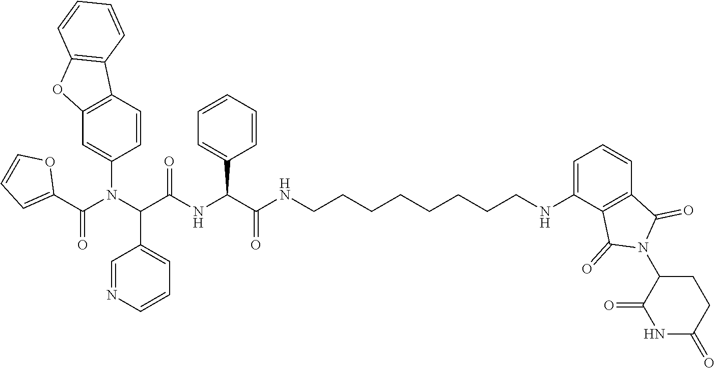 [SMILES: O=C1CCC(N2C(=O)c3cccc(NCCCCCCCCNC(=O)[C@@H](NC(=O)C(c4cccnc4)N(C(=O)c4ccco4)c4ccc5c(c4)oc4ccccc45)c4ccccc4)c3C2=O)C(=O)N1] | E | D | E |

Table 3. IC50 and antiviral data for compounds. Key: IC50: <0.1 uM=A, <1 uM=B, <10 uM=C, 10-25 uM=D, Not obtained or >25 uM=E. Antiviral Activity by cell viability assay: Not obtained=F, <0%=E, <25%=D, <50%=C, <75%=B, >75%=A

### REFERENCES

1) de Wispelaere, M., et al.; Nat Commun 10, 3468 (2019); https://doi.org/10.1038/841467-019-11429-w
2) Naoya Kitamura, et al; Journal of Medicinal Chemistry 2022 65 (4), 2848-2865; DOI: 10.1021/acs.jmedchem.1coo509
3) Zhang et al.; Science (2020) Vol 368, Issue 6489 pp. 409-412 DOI: 10.1126/science.abb3405
4) Andreas Luttens et al.; Journal of the American Chemical Society 2022 144 (7), 2905-2920; DOI: 10.1021/jacs.1co8402)

All publications mentioned in the above specification are herein incorporated by reference. Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A compound of formula (I): 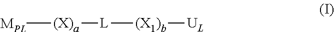
or a salt, solvate or tautomer thereof, wherein;
M_{PL} comprises a SARS-CoV-2 main protease ligand,
X comprises a divalent exit vector;
X1 comprises a divalent exit vector;
a and b are independently selected from 1 or 2;
L comprises a divalent linker, and
U_{L} comprises an E3 ubiquitin ligase ligand;
wherein M_{PL} is selected from: 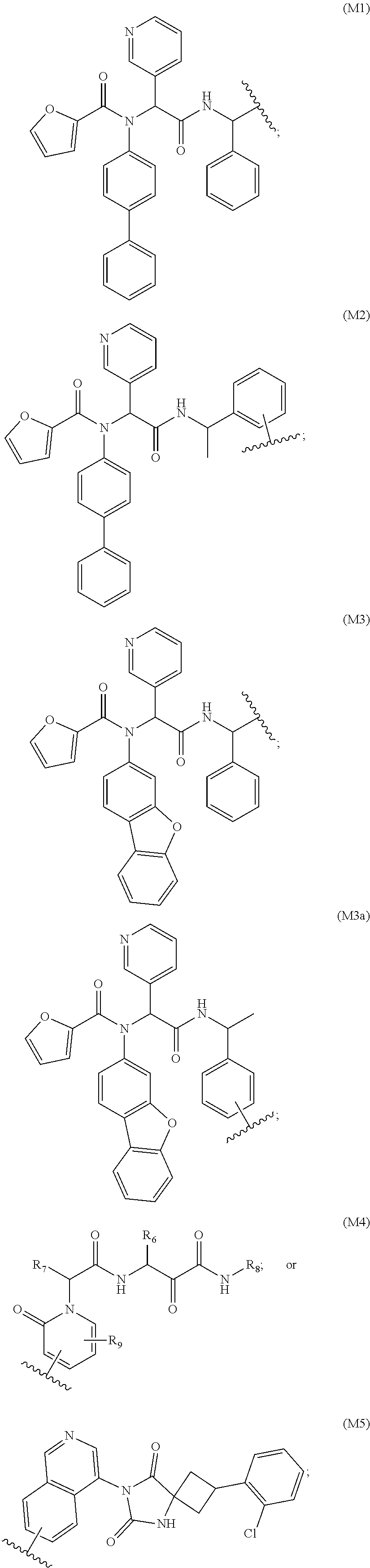
wherein:
R₆ is (CH₂)_{c}—R₁₄,
R₇ is (CH₂)_{c}—R₁₅,
R₈ is (CH₂)_{c}—R₁₈,
R₉ is selected from H, C₁ to C₆ alkyl, halo, cyclopropyl, or phenyl,
R₁₄ is selected from a lactam that may be unsubstituted or optionally substituted by 1 or 2 R₁₉ groups,
R₁₅ is selected from C₃ to C₆ cyclic alkyl or phenyl that may be unsubstituted or optionally substituted by 1 or 2 R₁₉ groups,
R₁₈ is selected from H, C₁ to C₆ alkyl, halo, cyclopropyl that may be unsubstituted or optionally substituted by a R₁₉ group, or phenyl that may be unsubstituted or optionally substituted by 1, 2 or 3 R₁₉ groups;
each c is independently 0, 1, or 2;
each R₁₉ is independently selected from C₁ to C₄ alkyl, or halo; and
the wavy line indicates the bond to X.

2. A compound as claimed in claim 1, wherein a is 1 and/or b is 1.

3. A compound as claimed in either claim 1 or claim 2, wherein U_{L} comprises an E3 ubiquitin ligase targeting ligand selected from a ligand that targets cereblon (CRBN), Von Hippel-Lindau (VHL), inhibitor of apoptosis protein (IAP), mouse double minute 2 homolog (MDM2), or Kelch like ECH associated protein 1 (KEAP1).

4. A compound as claimed in any one of the preceding claims, wherein U_{L} comprises a ligand selected from a univalent substituent derived from VH101, VH032, VH298 or an immunomodulatory imide drug (IMiD), optionally thalidomide, lenalidomide, pomalidomide, avadomide; or 3-phenylpiperidine-2,6-dione.

5. A compound as claimed in any one of the preceding claims, wherein U_{L} is selected from a species of the following formulae: 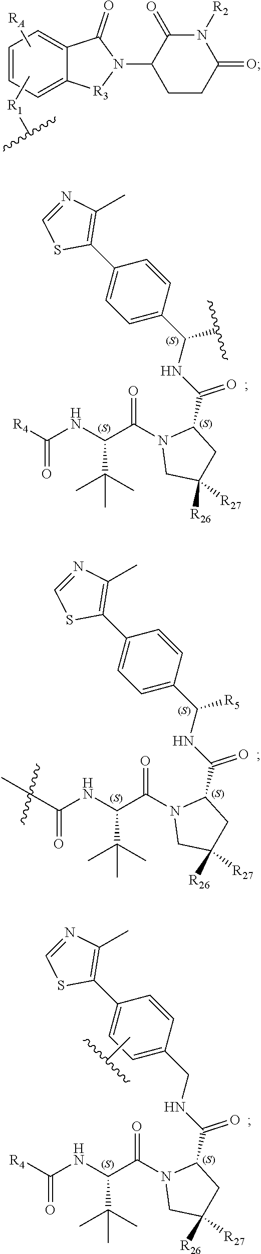 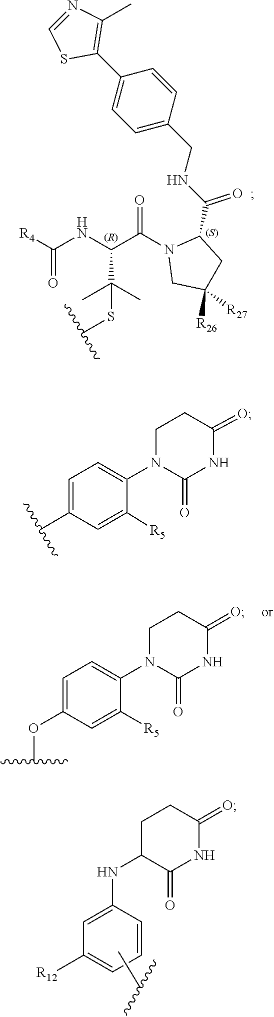
wherein R₁ is selected from —O—, —NH—, or —CH₂—, or is absent;
each R₂ or R₅ is independently selected from H or —CH₃;
each R₃ is independently selected from —CH₂— or —C(═O)—;
each R₄ is independently selected from —CH₃; 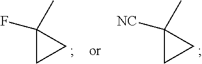
each R₂₆ or R₂₇ is selected from H, or OH; but R₂₆ and R₂₇ are not the same, R₁₂ is H or F,
each R_{A} is selected from H, C₁₋₆ alkyl, halo and (CH₂)ₜ—NR₂₀R₂₁,
t is selected from 0, 1, 2, or 3,
R₂₀ and R₂₁ are independently selected from H, and C₁₋₆ alkyl; and
the wavy line indicates the bond to X1.

6. A compound as claimed in any one of the preceding claims, wherein U_{L} is selected from a species of the following formulae: 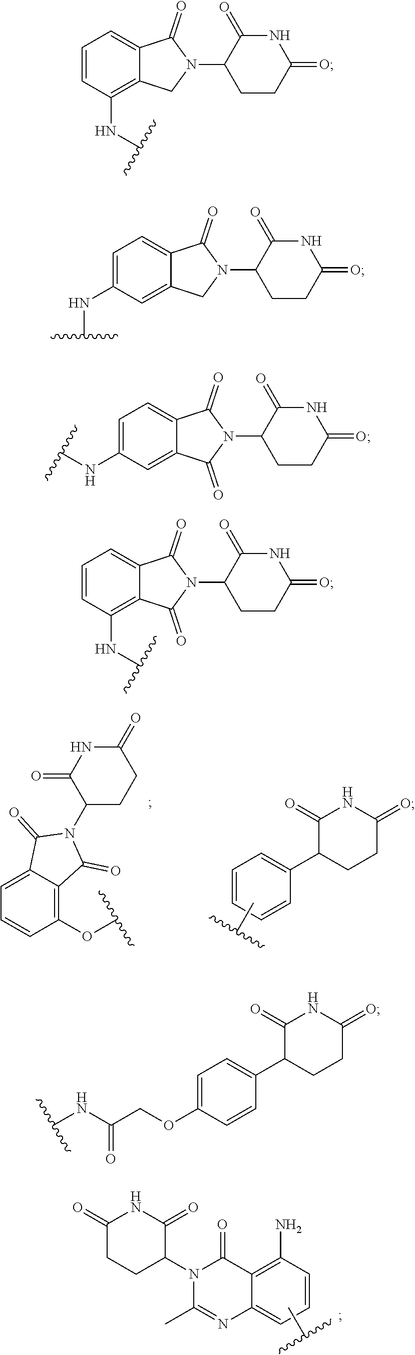 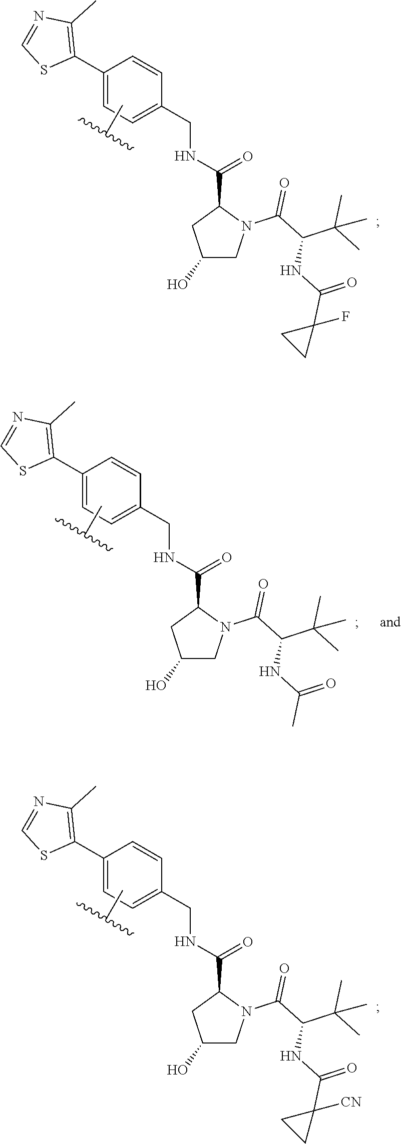
wherein R_{A} is selected from H, C₁₋₆ alkyl, halo and (CH₂)ₜ—NR₂₀R₂₁,
t is selected from 0, 1, 2, or 3,
R₂₀ and R₂₁ are independently selected from H, and C₁₋₆ alkyl; and
the wavy line indicates the bond to X1.

7. A compound as claimed in any one of the preceding claims, wherein each X is independently selected from a divalent substituent selected from a single covalent bond, —C(═O)—, —CH₂—, —O—, —S—, —C(═O)—NR₁₁—; —NR₁₁C(═O)—; —NR₁₁—; —C(═O)—O—; —O—CH₂—C(═O)—NR₁₁—; —NR₁₁—C(═O)—CH₂—O—; —C(═O)—CH₂—O—; 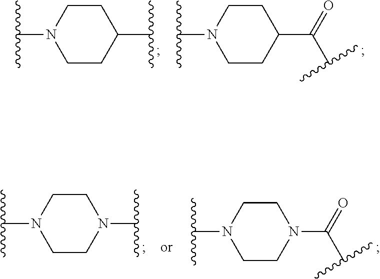
wherein each R₁₁ is independently selected from H, and C₁₋₆ alkyl, and
the bonds, or wavy lines in the formulae, indicate the bonds to M_{PL} and L or L and M_{PL}.

8. A compound as claimed in any one of the preceding claims, wherein L is a divalent linking group comprising 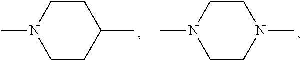
C₃₋₈ cycloalkylene, C₅₋₁₀ heteroarylene, phenylene, a C₁₋₁₂ alkylene chain which may contain one or more carbon-carbon double or triple bonds, a paraformaldehyde chain —(CH₂CH₂)ₙ(CH₂)ₘ(OCH₂)ₛ(CH₂CH₂)ₚ(CH₂)_{q}—, a polyethylene glycol chain —(CH₂CH₂)ₙ(CH₂)ₘ(OCH₂CH₂)ᵥ(CH₂CH₂)ₚ(CH₂)_{q}—, which chains may be interrupted by one, two or three groups selected from —O—, —S—, —NH—, halo, 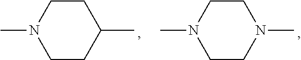
C₃₋₈ cycloalkyl, C₅₋₁₀ heteroarylene and/or phenylene;
wherein n, m, p and q are independently 0, 1 or 2,
s and v are independently 1 to 12, optionally 1 to 8, optionally 1 to 6.

9. A compound as claimed in any one of the preceding claims, wherein each X1 is independently selected from a single covalent bond, —C(═O)—, —CH₂—, —O—, —S—, —C(═O)—NR₁₁—; —NR₁₁C(═O)—; —NR₁₁—; —C(═O)—O—; —O—CH₂—C(═O)—NR₁₁—; —NR₁₁—C(═O)—CH₂—O—; —C(═O)—CH₂—O—; 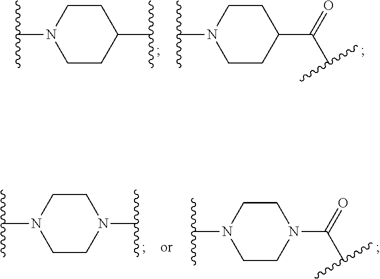
wherein each R₁₁ is independently selected from H, and C₁₋₆ alkyl, and the bonds, or wavy lines in the formulae, indicate the bonds to U_{L} and L or L and U_{L}.

10. A compound as claimed in any one of the preceding claims, wherein the compound is selected from compounds of the following formulae:

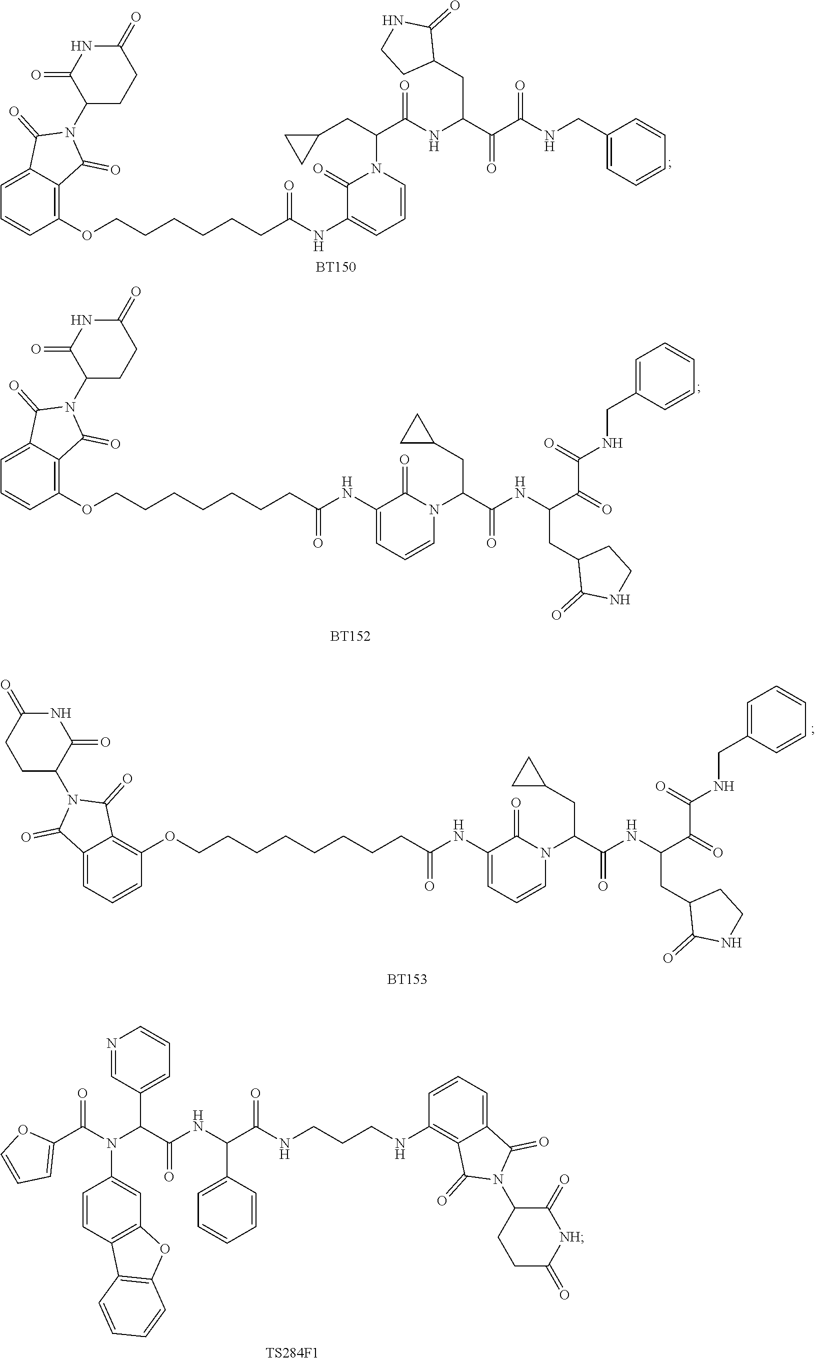

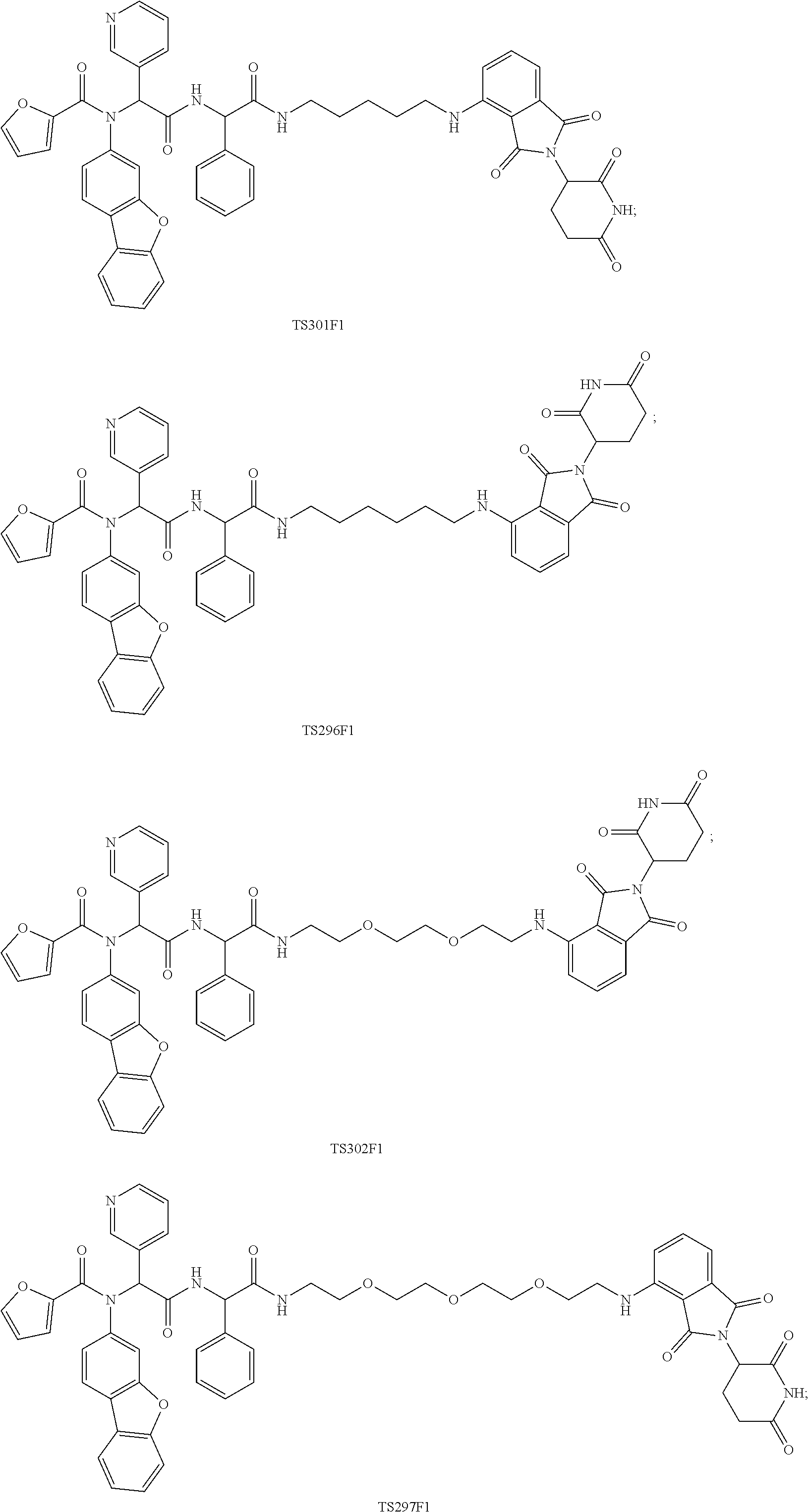

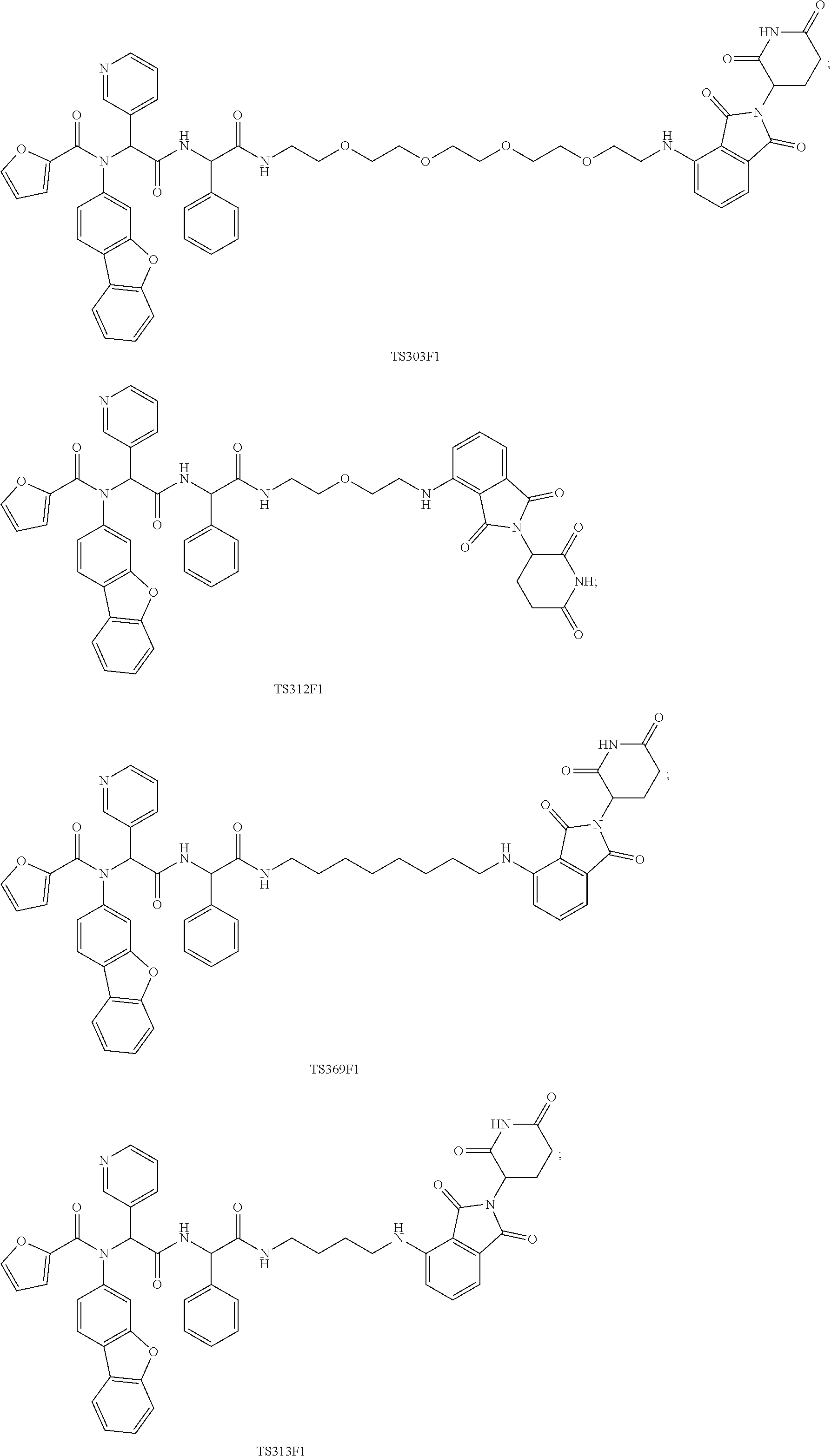

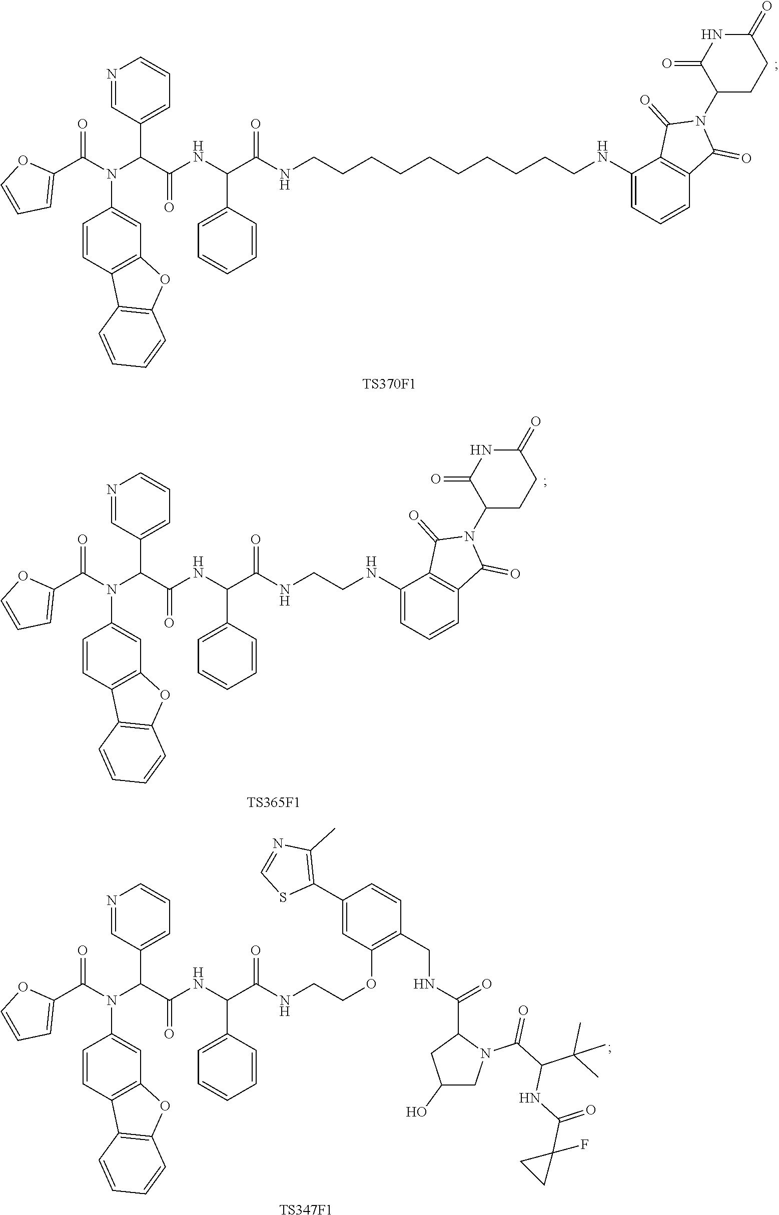

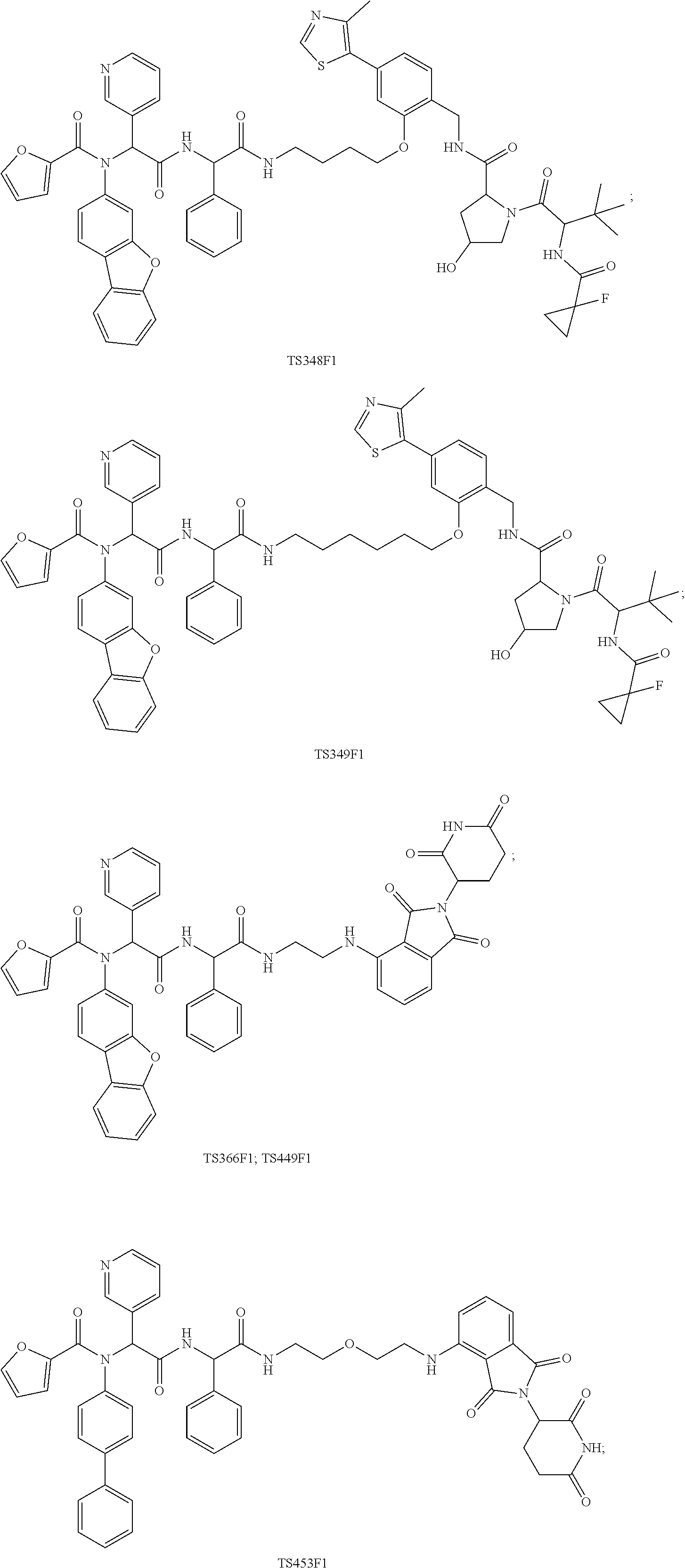

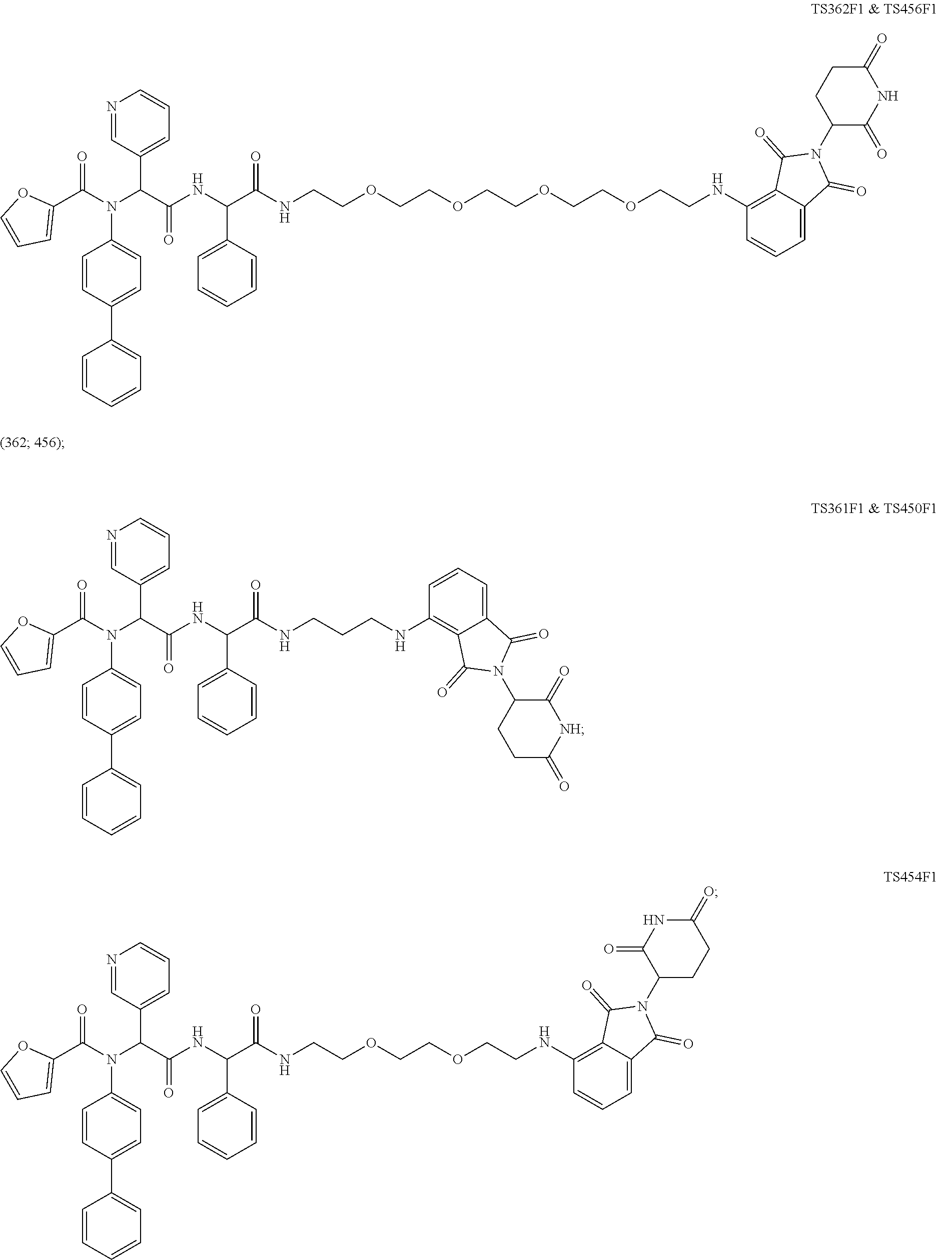 [SMILES: O=C1CCC(N2C(=O)c3cccc(NCCOCCOCCOCCOCCNC(=O)C(NC(=O)C(c4cccnc4)N(C(=O)c4ccco4)c4ccc(-c5ccccc5)cc4)c4ccccc4)c3C2=O)C(=O)N1 ; O=C1CCC(N2C(=O)c3cccc(NCCCNC(=O)C(NC(=O)C(c4cccnc4)N(C(=O)c4ccco4)c4ccc(-c5ccccc5)cc4)c4ccccc4)c3C2=O)C(=O)N1 ; O=C1CCC(N2C(=O)c3cccc(NCCOCCOCCNC(=O)C(NC(=O)C(c4cccnc4)N(C(=O)c4ccco4)c4ccc(-c5ccccc5)cc4)c4ccccc4)c3C2=O)C(=O)N1]

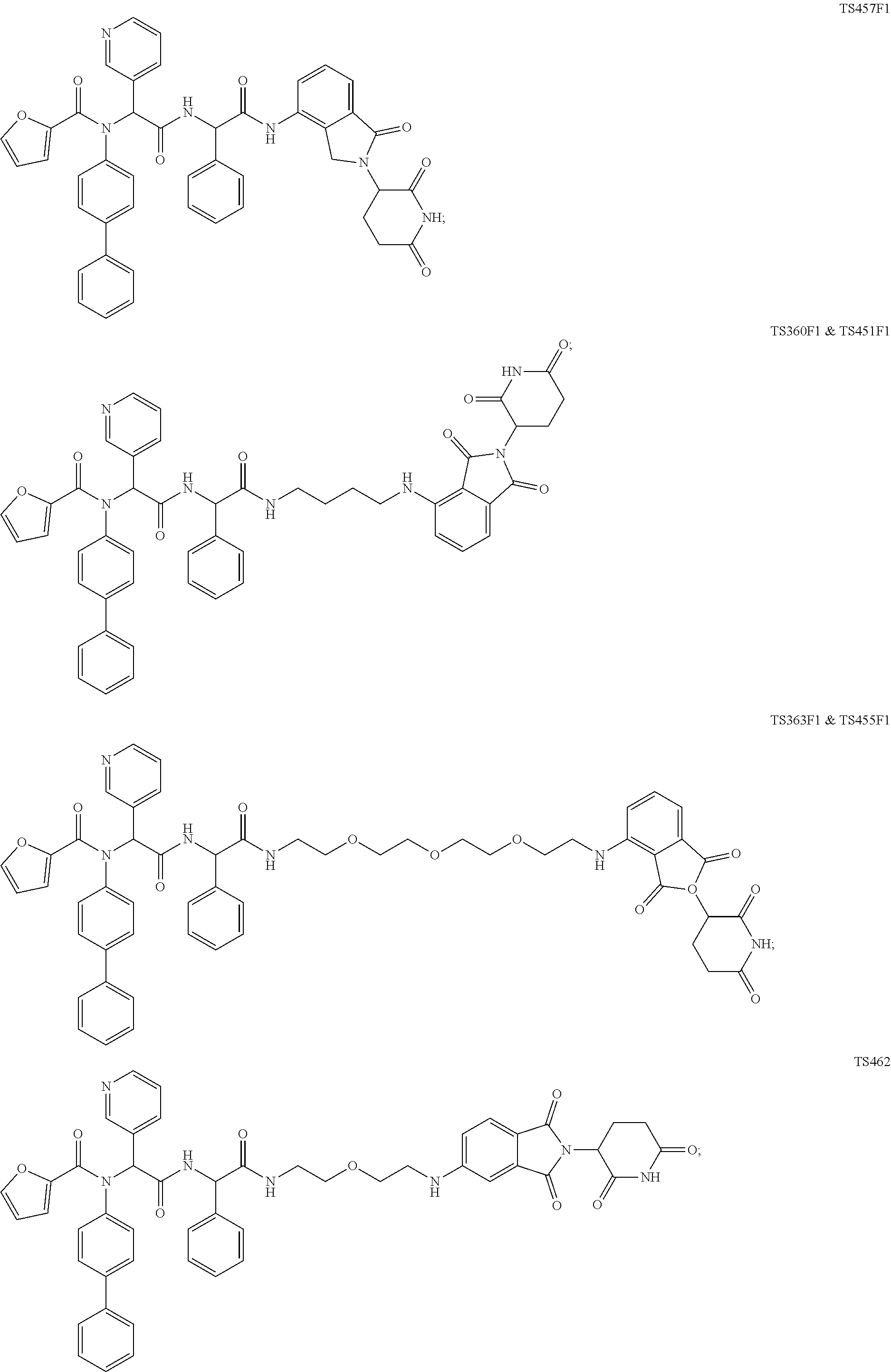

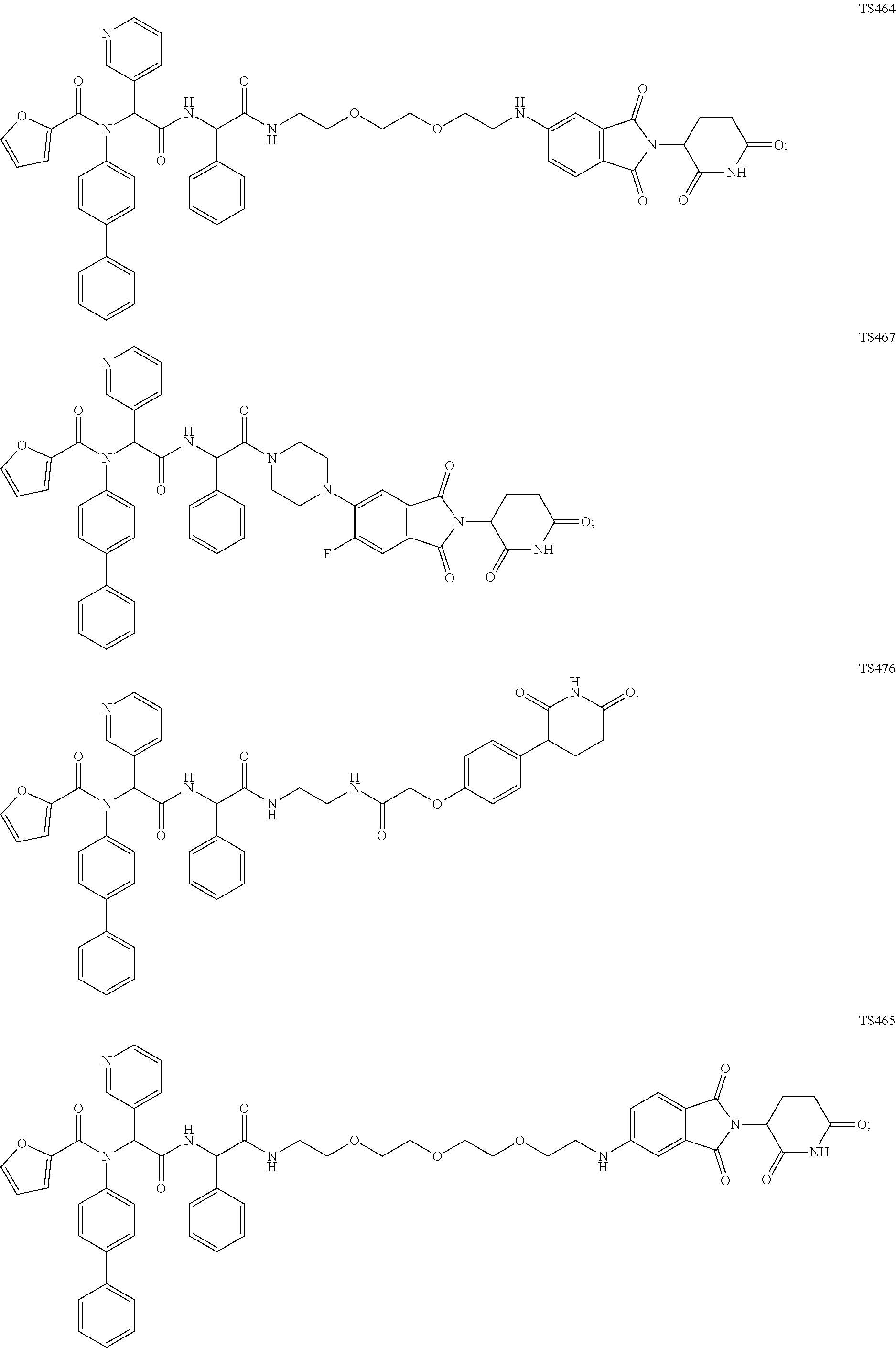

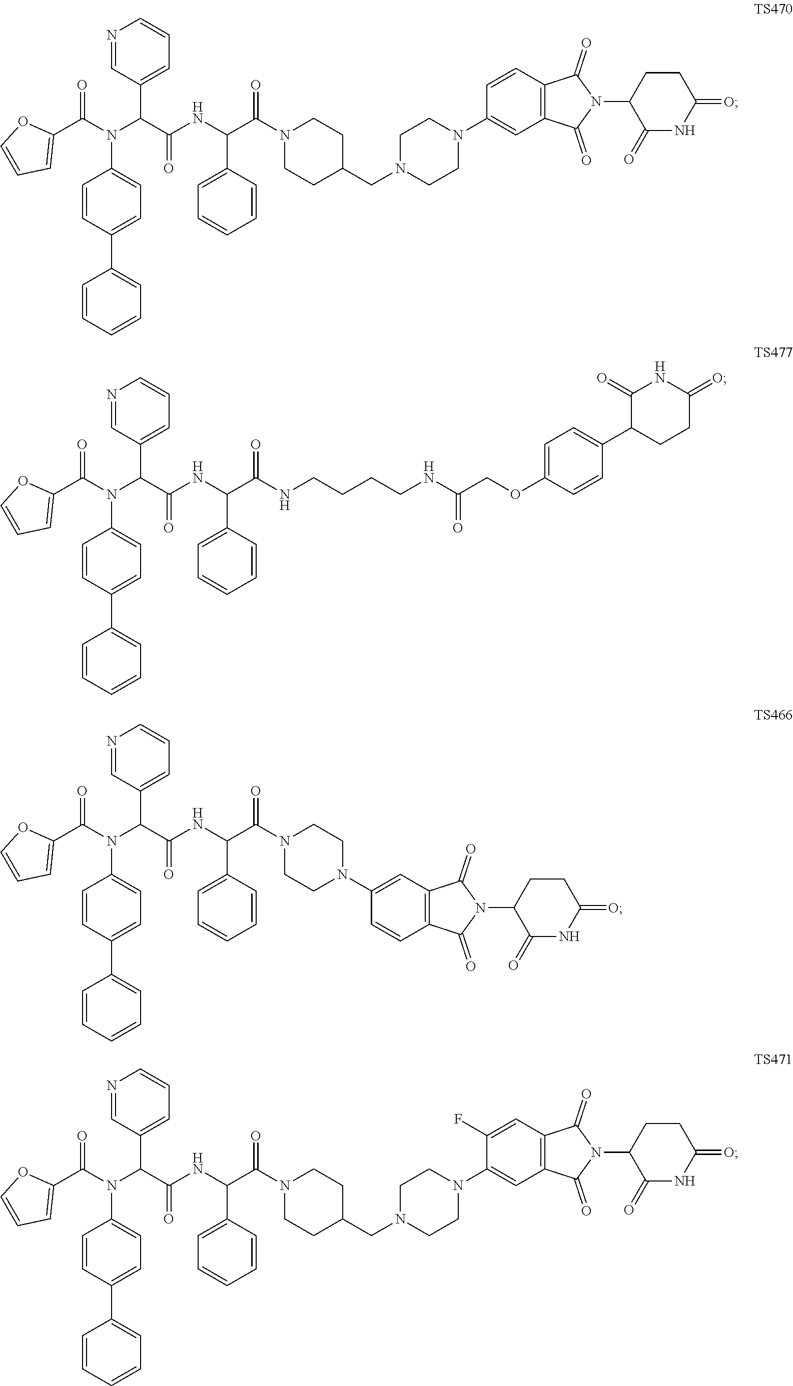

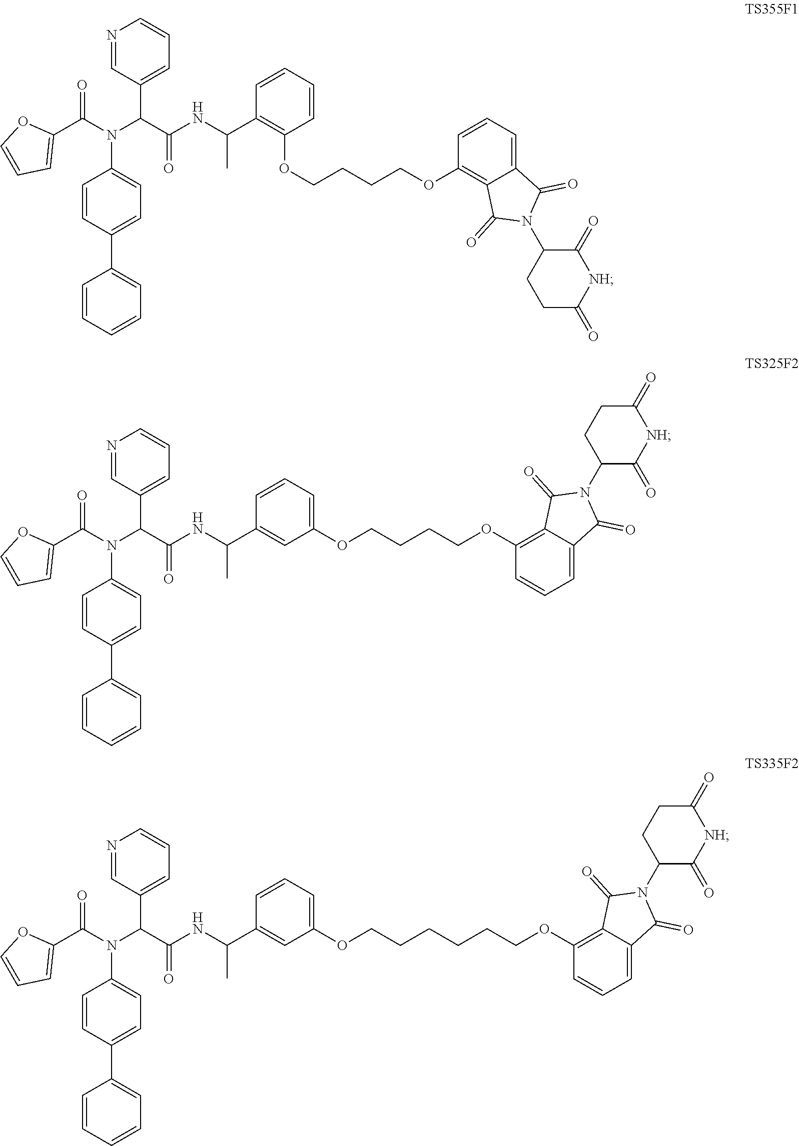 [SMILES: CC(NC(=O)C(c1cccnc1)N(C(=O)c1ccco1)c1ccc(-c2ccccc2)cc1)c1ccccc1OCCCCOc1cccc2c1C(=O)N(C1CCC(=O)NC1=O)C2=O ; CC(NC(=O)C(c1cccnc1)N(C(=O)c1ccco1)c1ccc(-c2ccccc2)cc1)c1cccc(OCCCCOc2cccc3c2C(=O)N(C2CCC(=O)NC2=O)C3=O)c1 ; CC(NC(=O)C(c1cccnc1)N(C(=O)c1ccco1)c1ccc(-c2ccccc2)cc1)c1cccc(OCCCCCCOc2cccc3c2C(=O)N(C2CCC(=O)NC2=O)C3=O)c1]

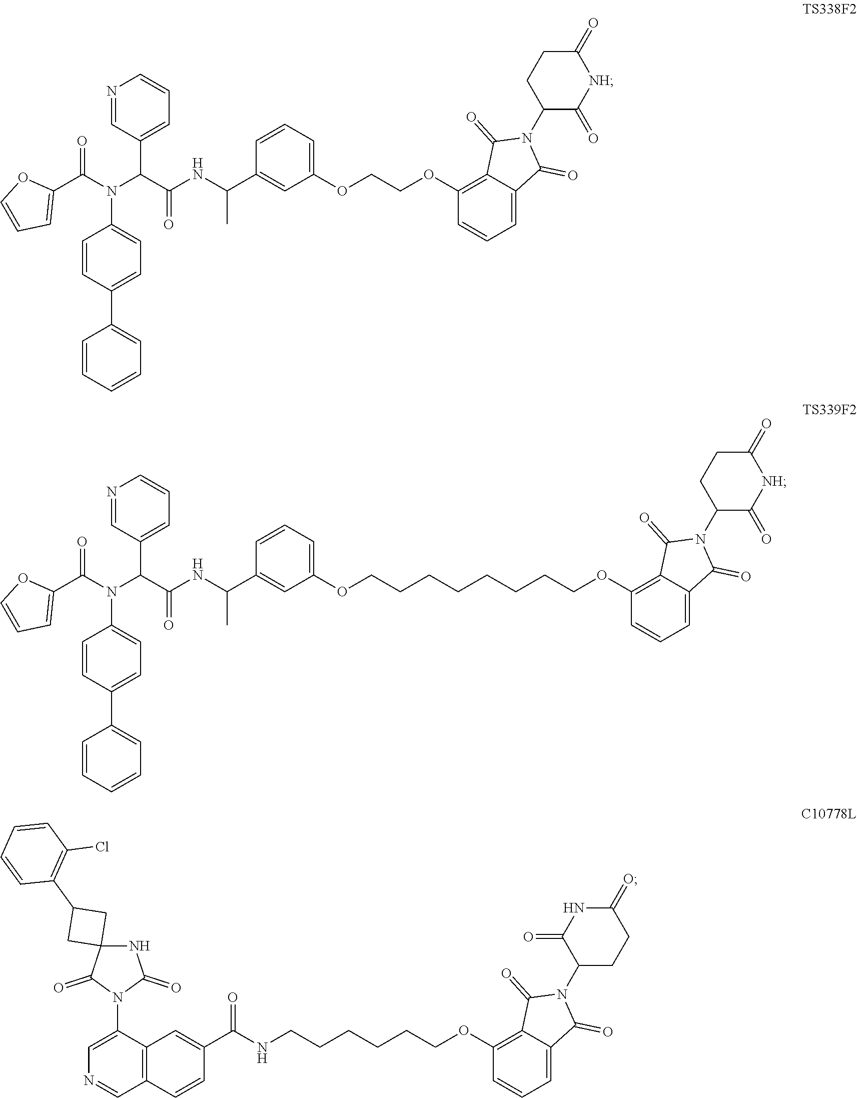 [SMILES: CC(NC(=O)C(c1cccnc1)N(C(=O)c1ccco1)c1ccc(-c2ccccc2)cc1)c1cccc(OCCOc2cccc3c2C(=O)N(C2CCC(=O)NC2=O)C3=O)c1 ; CC(NC(=O)C(c1cccnc1)N(C(=O)c1ccco1)c1ccc(-c2ccccc2)cc1)c1cccc(OCCCCCCCCOc2cccc3c2C(=O)N(C2CCC(=O)NC2=O)C3=O)c1 ; O=C1CCC(N2C(=O)c3cccc(OCCCCCCNC(=O)c4ccc5cncc(N6C(=O)NC7(CC(c8ccccc8Cl)C7)C6=O)c5c4)c3C2=O)C(=O)N1]

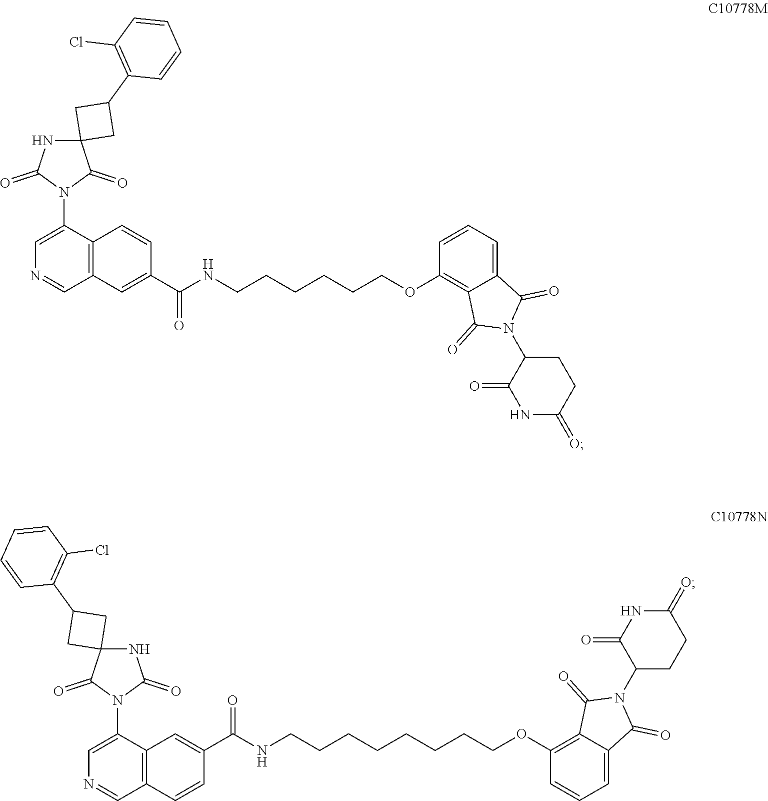 [SMILES: O=C1CCC(N2C(=O)c3cccc(OCCCCCCNC(=O)c4ccc5c(N6C(=O)NC7(CC(c8ccccc8Cl)C7)C6=O)cncc5c4)c3C2=O)C(=O)N1 ; O=C1CCC(N2C(=O)c3cccc(OCCCCCCCCNC(=O)c4ccc5cncc(N6C(=O)NC7(CC(c8ccccc8Cl)C7)C6=O)c5c4)c3C2=O)C(=O)N1]

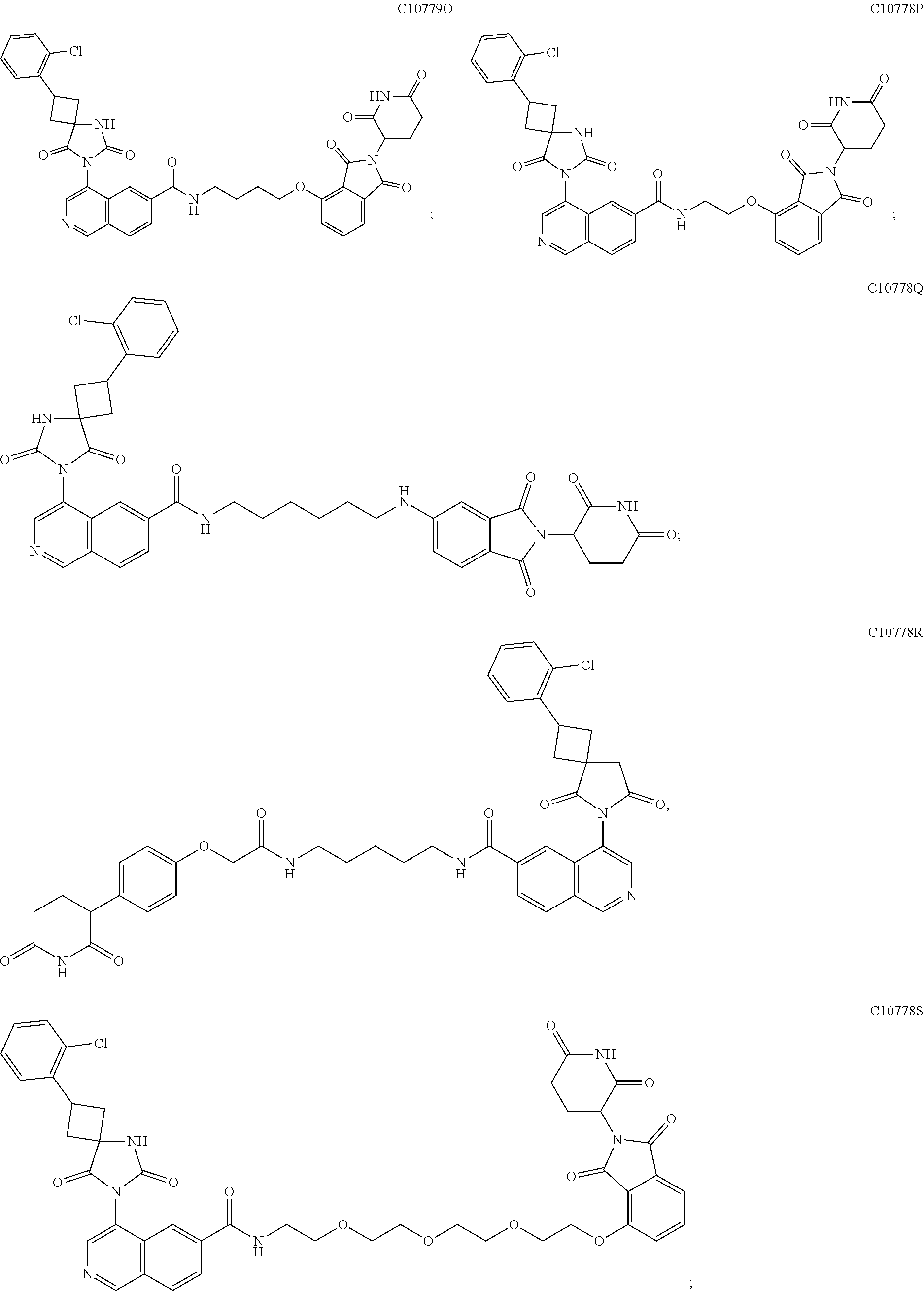 [SMILES: O=C1CCC(N2C(=O)c3cccc(OCCCCNC(=O)c4ccc5cncc(N6C(=O)NC7(CC(c8ccccc8Cl)C7)C6=O)c5c4)c3C2=O)C(=O)N1 ; O=C1CCC(N2C(=O)c3cccc(OCCNC(=O)c4ccc5cncc(N6C(=O)NC7(CC(c8ccccc8Cl)C7)C6=O)c5c4)c3C2=O)C(=O)N1 ; O=C1CCC(N2C(=O)c3ccc(NCCCCCCNC(=O)c4ccc5cncc(N6C(=O)NC7(CC(c8ccccc8Cl)C7)C6=O)c5c4)cc3C2=O)C(=O)N1 ; O=C(COc1ccc(C2CCC(=O)NC2=O)cc1)NCCCCCNC(=O)c1ccc2cncc(N3C(=O)CC4(CC(c5ccccc5Cl)C4)C3=O)c2c1 ; O=C1CCC(N2C(=O)c3cccc(OCCOCCOCCOCCNC(=O)c4ccc5cncc(N6C(=O)NC7(CC(c8ccccc8Cl)C7)C6=O)c5c4)c3C2=O)C(=O)N1]

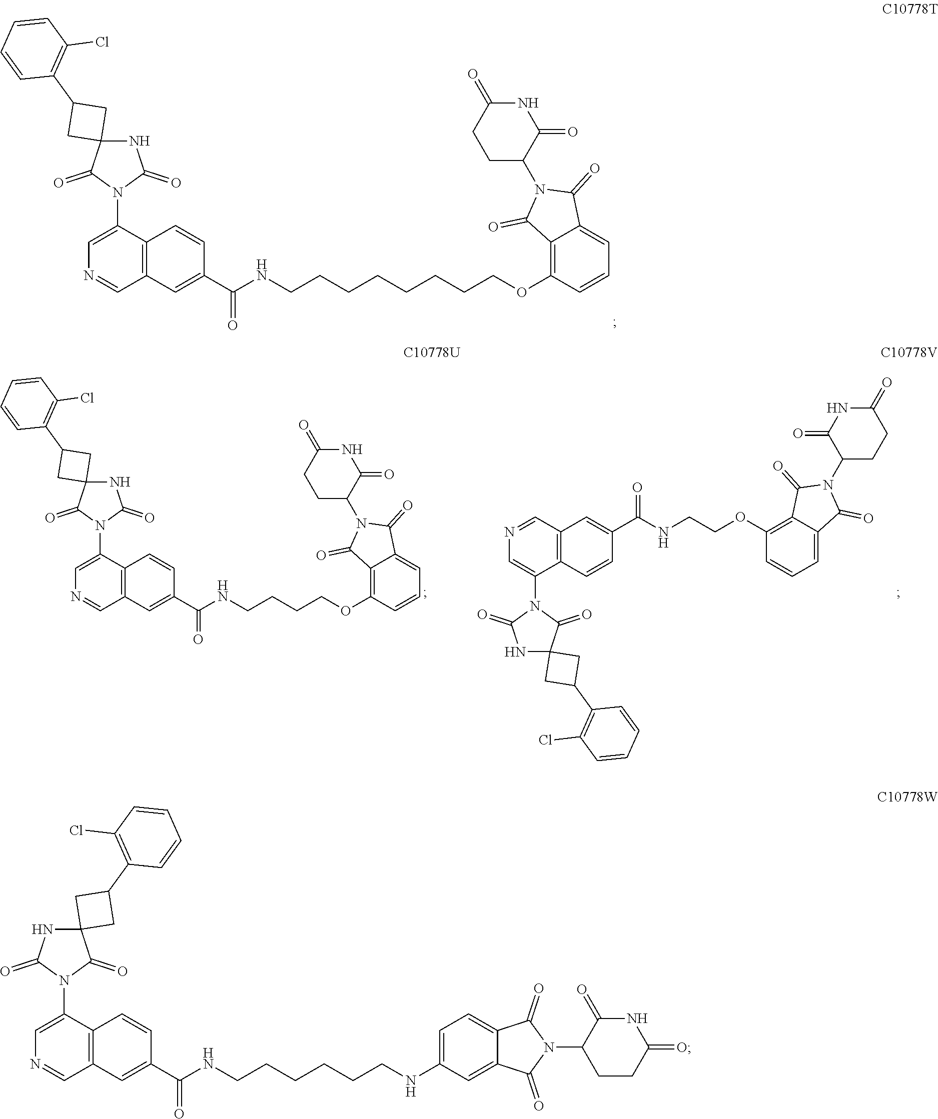 [SMILES: O=C1CCC(N2C(=O)c3cccc(OCCCCCCCCNC(=O)c4ccc5c(N6C(=O)NC7(CC(c8ccccc8Cl)C7)C6=O)cncc5c4)c3C2=O)C(=O)N1 ; O=C1CCC(N2C(=O)c3cccc(OCCCCNC(=O)c4ccc5c(N6C(=O)NC7(CC(c8ccccc8Cl)C7)C6=O)cncc5c4)c3C2=O)C(=O)N1 ; O=C1CCC(N2C(=O)c3cccc(OCCNC(=O)c4ccc5c(N6C(=O)NC7(CC(c8ccccc8Cl)C7)C6=O)cncc5c4)c3C2=O)C(=O)N1 ; O=C1CCC(N2C(=O)c3ccc(NCCCCCCNC(=O)c4ccc5c(N6C(=O)NC7(CC(c8ccccc8Cl)C7)C6=O)cncc5c4)cc3C2=O)C(=O)N1]

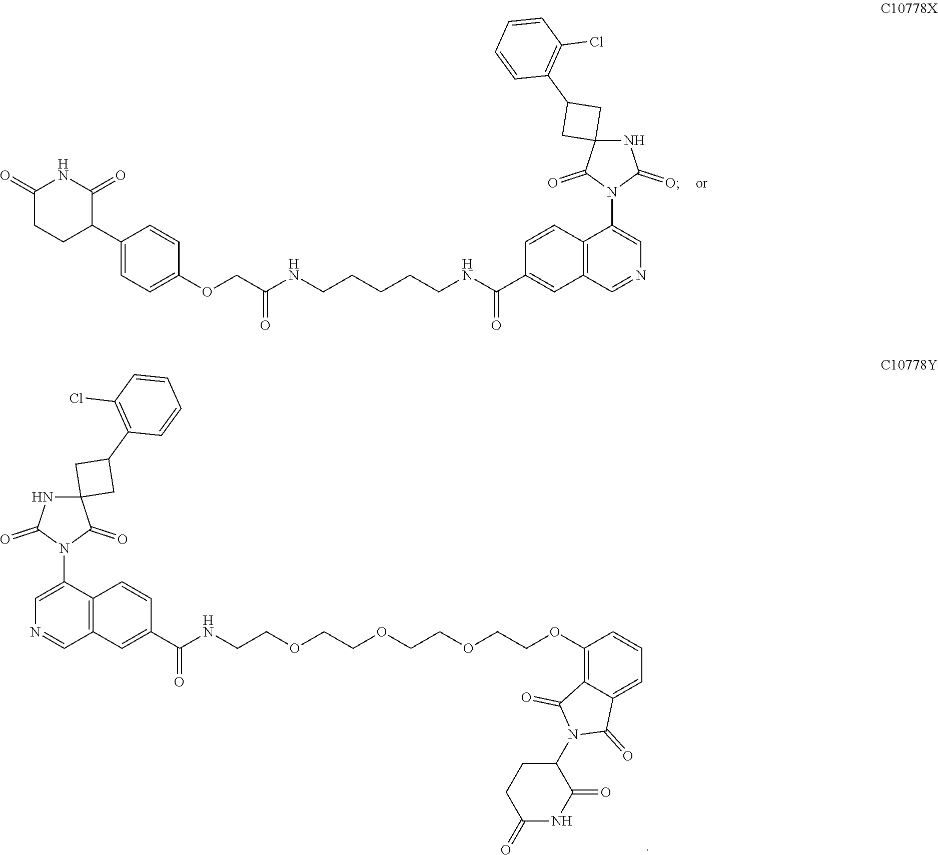 [SMILES: O=C(COc1ccc(C2CCC(=O)NC2=O)cc1)NCCCCCNC(=O)c1ccc2c(N3C(=O)NC4(CC(c5ccccc5Cl)C4)C3=O)cncc2c1 ; O=C1CCC(N2C(=O)c3cccc(OCCOCCOCCOCCNC(=O)c4ccc5c(N6C(=O)NC7(CC(c8ccccc8Cl)C7)C6=O)cncc5c4)c3C2=O)C(=O)N1]

11. A compound of any one of claims 1 to 10, or a pharmaceutically acceptable salt thereof, for use as a medicament.

12. A compound of any one of claims 1 to 10, or a pharmaceutically acceptable salt thereof, for use as a medicament in the treatment of a viral disease.

13. A compound of any one of claims 1 to 10, or a pharmaceutically acceptable salt thereof, for use as a medicament in the treatment of a coronavirus disease.

14. A compound of any one of claims 1 to 10, or a pharmaceutically acceptable salt thereof, for use as a medicament in the treatment of SARS-CoV-2.

15. A combination comprising a compound of any one of claims 1 to 10, or a pharmaceutically acceptable salt thereof, and at least one other active agent.

16. A pharmaceutical composition comprising a compound of any one of claims 1 to 10, or a combination as claimed in claim 15, and a pharmaceutically acceptable excipient, carrier or diluent.

17. A method of treatment of a subject suffering from a viral disease, the method comprising administering to said subject a therapeutically effective amount of a compound of any one of claims 1 to 10 or a pharmaceutical composition of claim 16.

18. A method comprising providing a composition comprising a compound of any one of claims 1 to 10, and contacting the composition with a source of a viral protease, optionally corono-viral protease.

19. A method comprising providing a composition comprising a compound of any one of claims 1 to 10, and contacting the composition with a source of SAR-CoV-2 Mpro.

20. A reagent comprising a compound of any one of claims 1 to 10 and a solvent.
